(19) United States
(12) Patent Application Publication
(10) Pub. No.: US 2014/0255424 A1
(43) Pub. Date: Sep. 11, 2014

(54) BIOMARKERS OF AGING FOR DETECTION AND TREATMENT OF DISORDERS

(71) Applicants: The Board of Trustees of the Leland Stanford Junior University, Palo Alto, CA (US); Department of Veterans Affairs, Washington, DC (US)

(72) Inventors: Anton Wyss-Coray, Stanford, CA (US); Thomas A. Rando, Stanford, CA (US); Markus Britschgi, Allschwil (CH); Kaspar Rufibach, Bern (CH); Saul Abraham Villeda, Mountain View, CA (US)

(73) Assignees: The Board of Trustees of the Leland Stanford Junior University, Palo Alto, CA (US); Department of Veterans Affairs, Washington, DC (US)

(21) Appl. No.: 14/280,939

(22) Filed: May 19, 2014

Related U.S. Application Data
(63) Continuation of application No. 13/575,437, filed on Oct. 9, 2012.
(60) Provisional application No. 61/298,998, filed on Jan. 28, 2010.

(51) Int. Cl. G01N 33/68; C07K 16/24; C07K 16/18; C12N 15/113; C12Q 1/68

(52) U.S. Cl. CPC G01N 33/6896; C12N 15/113; C12Q 1/6883; C07K 16/18; C07K 16/24
USPC 424/158.1; 514/44 A; 436/501; 435/6.12; 506/9; 435/7.92; 435/7.21; 424/172.1

(57) ABSTRACT

Provided are methods of diagnosis, prognosis, and monitoring of aging using biomarkers that have been discovered to be linked to biological aging process. Methods for increasing neural cell regeneration and cognitive function are also provided. The methods are, at least in part, based on a discovery that altered expression patterns of certain biological markers are associated with biological aging processes. These markers comprise at least Eotaxin/CCL11, β2-microglobulin, MCP-1 and Haptoglobulin, increased expression of which has been shown to be associated with increase in biological aging process.

### CROSS-REFERENCE TO RELATED APPLICATIONS

This is a Continuation Application of U.S. Ser. No. 13/575,437, filed on Oct. 9, 2012, which is a 35 U.S.C. §371 National Phase Entry Application of International Application No. PCT/US2011/022916, filed Jan. 28, 2011, which designates the United States, and which claims benefit under 35 U.S.C. §119(e) of the U.S. provisional application No. 61/298,998, filed on Jan. 28, 2010, the contents of which are herein incorporated by reference in their entirety.

### GOVERNMENT SUPPORT

This invention was made with Government support under contracts AG027505 and OD000392 awarded by the National Institutes of Health. The Government has certain rights in this invention.

### BACKGROUND

Aging is related to some of the most prevalent diseases in modern society including cardiovascular disease, cancer, arthritis, dementia, cataract, osteoporosis, diabetes, hypertension, stroke, and Alzheimers disease (AD). The incidence of all of these age-associated diseases increases rapidly with chronological age but is also associated with premature biological aging due to environmental and genetic factors. For example, the incidence of cancer increases exponentially with age. Currently, the knowledge of the age-related biological processes including those that are involved in these diseases is still limited, and effective treatment for many of these age-associated diseases is still not available. There is a need to develop simple, non-invasive tests to diagnose, treat and monitor age-related changes and age-associated disorders or diseases using biomarkers that provide signatures related to biological aging process. Methods for alleviating the age-related deterioration in learning and memory would also be useful.

One hallmark of aging is diminished tissue regeneration. The regenerative properties of most tissues gradually decline with age, mainly due to the age-associated declined activity of tissue-specific stem cells. Particularly in the central nervous system (CNS), aging results in a decline in adult neural stem cell/progenitor cells (NPCs) and neurogenesis, with subsequent impairments in olfaction and cognitive functions such as learning and memory. Adult neurogenesis occurs in local microenvironments, or neurogenic niches, which is localized around blood vessels. Emerging evidence using three-dimensional imaging techniques has also suggested that neurovascular interactions in the neurogenic niche may have a functional significance. Hence contacts between NPCs and blood vessels are permeable, denude of astrocytic endfeet and a pericyte sheath, which may indicate an intact blood-brain-barrier. The absence of a classical BBB potentially enables circulating molecules from the periphery to access the neurogenic niche. To date while adult NPC populations and their respective microenvironments have been characterized, little is known about both the intrinsic and extrinsic regulation of NPCs during the aging process. In particular, little is known as to if and how changes of the systemic environment (e.g., cues extrinsic to the CNS delivered by blood) to the molecular composition of the neurogenic niche can alter and/or impair and/or improve NPC function during aging. It is unclear if systemic factors can be used to correlate with declined NPC functions and neurogenesis during aging, and to regulate NPC functions and neurogenesis. Knowledge of these processes would be helpful to diagnosis and treatment of age-associated deterioration in learning speed and memory as well as age-associated disorders or diseases, in particular CNS related disorders or diseases.

### SUMMARY OF THE INVENTION

The present application provides methods of diagnosis, prognosis and monitoring of altered neural cell regenerative capacity and/or altered cognitive function using biomarkers that have been linked to biological aging process. The methods are, at least in part, based on a discovery that altered expression patterns of certain biological markers are associated with biological aging processes. These markers comprise at least one or more of the following proteins: Eotaxin/CCL11, β2-microglobulin, MCP-1 and Haptoglobulin, increased expression of which we have shown to be associated with increase in biological aging process.

Accordingly, the invention provides a method for measuring altered neural cell regenerative capacity and/or altered cognitive function in a subject the method comprising analyzing in a biological sample the amount of at least one biomarker from a group of four proteins consisting of CCL11, haptoglobin, CCL2, and β2-microglobin, wherein increase of about or more than 50% or alternatively about or more than 2-fold in the amount of the at least one protein compared to a reference value is indicative of decreased regenerative capacity and cognitive function in the subject.

In some aspects, the method further comprised a step of administering to the subject diagnosed with decreased neural cell regenerative capacity and cognitive function, an anti-inflammatory agent, such as a non-steroidal anti-inflammatory drug (NSAID), for example aspirin, ibuprofen, or naproxen.

In some aspects the method further comprises administering to the subject with decreased neural cell regenerative capacity and cognitive function, an antagonist of the receptor to which the biomarker binds. Some non-limiting examples of such receptors include β2-microglobulin receptors, such as major histocompatibility complex (MHC) class I proteins; Haptoglobin receptors, such as CD163; CCL2 receptods, such as CCR2, D6, DARC; and CCL11 receptors, such as CCR3, CCR5, D6, DARC. Such receptors are described, for example, in Allen et al., Annu. Rev. Immunol. 25: 787-820, 2007, incorporated herein by reference. Receptor antagonists, such as antibodies, decoys, small molecules, peptides, and like can be used.

In some aspects the receptor antagonist is CCR2 antagonist. In some aspects, the antagonist is CCR3 antagonist. In some embodiments, a combination of the CCR2 and CCR3 antagonists are used.

Several of such receptor antagonists are already known. For example, CCR2 antagonists include a CCR2 antagonist CAS Number: 445479-97-0 with molecular formula C₂₈H₃₄F₃N₅O₄S, CCR2 antagonists made by Ingenta, indicated with an identifier INCB8696. Quaternary salt CCR2 antagonists are described in U.S. Pat. No. 7,799,824 (incorporated herein by reference in its entirety); and aryl sulfonamide derivatives, described, e.g., in U.S. Pat. No. 7,622,583 (incorporated herein by reference in its entirety). Examples of CCR3 antagonists useful according to the methods of the invention include bipiperdine derivatives described in U.S. Pat. No. 7,705,153 (incorporated herein by reference in its entirety); and cyclic amine CCR3 antagonists described in U.S. Pat. No. 7,576,117 (incorporated herein by reference in its entirety).

In some aspects the method further comprises a step of administering to the subject diagnosed with decreased regenerative capacity and cognitive function, a neutralizing antibody or RNA interfering agent against the biomarker the amount of which is increased.

In some aspects of the method, the amount of at least two proteins from the four are analyzed. In some aspects, the two proteins are CCL11 or CCL-2.

In some aspects, when the subject is human, the reference value is a value derived from pooled sample of humans between 20 and 45 years old who have been diagnosed as not being affected with impaired cognitive function. The reference value is typically matched with the type of fluid to be analyzed. For example, if the analyzed fluid is plasma, the reference value is from plasma sample, if it is from cerebrospinal fluid, the reference value is also from cerebrospinal fluid. The reference value can also be a value from a average age-matched samples or a value from age-matched pooled samples. The reference value can be a value that is determined earlier or a value that is determined from a control sample analyzed in parallel with the test sample. The reference value can also be a panel of values, ranging from values from young to old samples, such as samples from 20-25 yr old humans, 25-30, 30-35 and so forth. In some aspects, the reference value can also be gender matched.

In some aspects, the biological sample is a peripheral fluid sample, such as blood, serum, plasma, cerebrospinal fluid, or urine. Other fluid samples such as lymph, sputum, and tears can also be used.

In one embodiment, the invention provides a method of identifying an agent capable of increasing decreased regenerative capacity and/or cognitive function the method comprising administering to a test animal over-expressing one or more of the group of proteins consisting of CCL11, haptoglobin, CCL2, and β2-microglobin, a test agent, and analyzing whether the amount of the protein is decreased compared to the level of the protein prior to administration of the test agent, wherein if the amount of the protein id decreased, the test agent is identified as an agent is capable of increasing regenerative capacity and/or cognitive function.

In some aspects, the decreased regenerative capacity or cognitive function is associate with a neurodegenerative disease, such as Alzheimer's disease, Parkinson's disease, Amyotrophic lateral sclerosis or neuroinflammatory disease.

In some aspects, the subject is a human subject. In some aspects the subject is a non-human subject. In some aspects, the subject is a non-human mammal.

In some aspects, the amount or level of the biomarker is determined using an assay measuring the protein amount, such as using an antibody-based detection method in an immunoassay, or the mRNA amount, such as using any one of the well known quantitative PCR methods.

The invention also provides a system comprising a determination module configured to receive and output a measuring information indicating the presence or level of a biomarker selected from a group comprising at least one protein from the group of four proteins consisting CCL11, haptoglobin, CCL2, and β2-microglobin from the biological fluid sample of a subject; a storage assembly configured to store output information from the determination module; a comparison module adapted to compare the data stored on the storage module with at least one reference value, and to provide a comparison content, wherein if the reference value is two fold or more different from the input information, the comparison module provides information to the output module that the biological fluid sample is associated with a subject that deviates from the reference value; and an output module for displaying the information for the user.

In one embodiment, the invention provides for methods of diagnosing an age-associated disorder in a subject, the method comprising comparing a level of at least one biomarker in a biological fluid sample from the subject to a reference level of said at least one biomarker from a population of healthy subjects without said age-associated disorder of the chronological age matched group, wherein an increased level of said biomarker from said subject compared to said reference level indicates a diagnosis of the age-associated disorder in said subject. The method of diagnosing the age-associated disorder in the subject may further comprise a step of administering a neutralizing antibody against the biomarker.

In one embodiment, provided herein is a method of diagnosing neuroinflammation in a subject, the method comprising comparing a level of at least one biomarker in a biological fluid sample from the subject to a reference level of said at least one biomarker from a population of healthy subjects without neuroinflammation of the chronological age matched group, wherein an increased level of said at least one biomarker from said subject compared to said reference level indicates a diagnosis of neuroinflammation in said subject. The method may further comprise a step of administering an anti-inflammatory agent to the subject diagnosed with neuroinflammation.

In some embodiments, provided herein are methods for detecting diminished cell activity in a subject, the method comprising comparing a level of at least one biomarker in a biological fluid sample from the subject to a reference level of said at least one biomarker from a population of healthy subjects having normal cell activity of the chronological age matched group, wherein an increased level of said at least one biomarker from said subject compared to said reference level indicates a diminished cell activity in said subject.

In some embodiments, provided herein are methods for detecting diminished tissue regeneration capacity in a subject, the method comprising comparing a level of at least one biomarker in a biological fluid sample from the subject to a reference level of said at least one biomarker from a population of healthy subjects having normal tissue regeneration activity of the chronological age matched group, wherein an increased level of said at least one tissue regeneration capacity-associated biomarker from said subject compared to said reference level indicates a diminished tissue regeneration capacity in said subject.

In another aspect, the present invention provides for methods for identifying a medical treatment or medication for a subject for promoting cell activity, increasing tissue regeneration capacity or treating an age-associated disorder or disease for a subject, the method comprising comparing at a later time point a level of at least one biomarker in a biological fluid sample from said subject exposed to said medical treatment or medication to the level of said at least one biomarker from said subject at an earlier time point, wherein a decreased level of said at least one biomarker at the later time point compared to the earlier time point indicates a suitable medical treatment or medication for promoting cell activity, increasing tissue regeneration capacity or treating said age-associated disorder for said subject.

In yet another aspect, the present invention provides for methods for identifying a medical treatment or medication for promoting cell activity, increasing tissue regeneration capacity or treating an age-associated disorder or disease for a population of subjects, the method comprising comparing at a later time point a level of at least one biomarker in biological fluid samples from a population of subjects exposed to said medical treatment or medication to the level of said at least one biomarker from said population of subjects at an earlier time point, wherein a decreased level of said at least one biomarker at the later time point compared to the earlier time point indicates a suitable medical treatment or medication for promoting cell activity, increasing tissue regeneration capacity or treating said age-associated disorder.

Another aspect of the present invention relates to methods of monitoring the effect of a medical treatment or a medication on a subject for promoting cell activity, increasing tissue regeneration capacity or treating an age-associated disorder, the method comprising comparing at a later time point a level of at least one biomarker in a biological fluid sample from said subject exposed to said medical treatment or medication to the level of said at least one biomarker from said subject at an earlier time point, wherein a decreased level of said at least one biomarker at the later time point compared to the earlier time point indicates an effective medical treatment or medication on said subject for promoting cell activity, increasing tissue regeneration capacity or treating said age-associated disorder.

A further aspect of the present invention provides for methods of screening for candidate agents for the treatment of age-associated disorders or diseases by identifying candidate agents for activity in modulating age-associated disorders/diseases biomarkers. Thus some embodiments of the present invention provides for methods of identifying a candidate agent for modulating the activity or expression of a biomarker selected from the group consisting of Eotaxin/CCL11, β2-microglobulin, MCP-1 and Haptoglobin, the method comprising contacting said candidate agent in an assay; detecting the expression or activity of said biomarker; and comparing the expression or activity of said biomarker to a reference level of said biomarker, wherein an decreased expression or activity of said biomarker indicates an inhibition of the expression or activity of said biomarker by said candidate agent, and wherein an increased expression or activity of said biomarker indicates a promotion of the expression or activity of said biomarker by said candidate agent.

Provided herein are also methods of screening for receptors or ligands that can bind to the age-associated disorders/diseases biomarkers. Some embodiments relate to methods of identifying a receptor for a biomarker selected from the group consisting of Eotaxin/CCL11, β2-microglobulin, MCP-1 and Haptoglobin, said method comprising contacting a cell transfected with a nucleic acid encoding a candidate receptor with the biomarker under conditions suitable for binding, and detecting specific binding of the biomarkers to the candidate receptor, wherein binding to the candidate receptor is indicative of a receptor for the biomarker. By utilizing antagonists to the identified receptors to the biomarkers, activity of the biomarkers can be modulated, and hence eventually achieving the treatment of age-associated disorders or diseases.

In one aspect of the present invention, provided is a kit comprising at least one reagent specific to at least one biomarker, and may further include instructions for carrying out a method described herein. In some embodiments, the present invention provides for a kit comprising at least one reagent specific to at least one age-associated biomarker, said at least one biomarker selected from the group consisting of Eotaxin/CCL11, β2-microglobulin, MCP-1, and Haptoglobin; and instructions for carrying out any of the method described above in the present invention.

In another aspect, the present invention provides for a device comprising a measuring assembly yielding detectable signal from an assay indicating the presence or level of an age-associated biomarker from the biological fluid sample of an individual; and an output assembly for displaying an output content for the user.

In one embodiment, the invention provides a method of slowing aging process in a subject, such as a human, the method comprising administering to the subject an agonist of a protein selected from CCL2/MCP-1 and CCL11/Eotaxin. In some aspects, the subject is over 45 years old. In some aspects, the subject is affected with diagnosed cognitive impairment or an age-associated disease.

### BRIEF DESCRIPTION OF THE DRAWINGS

FIGS. 1A-1E show that heterochronic parabiosis reduces adult neurogenesis in young animals while increasing neurogenesis in aged mice. FIG. 1A shows a schematic of the three combinations of mice used in isochronic and heterochronic pairings. FIG. 1B shows quantification of neurogenesis in the young DG after parabiosis. Data are from 12 mice for isochronic and 10 mice for heterochronic groups (5-7 sections per mouse). FIG. 1C shows quantification of neurogenesis in the old DG after parabiosis. Data are from 6 mice for isochronic and 12 mice for heterochronic groups (5-7 sections per mouse; **, P<0.01). e, High magnification view of neurite arbors from Doublecortin-positive neurons from young (scale bar: 50 μm) and old (scale bar: 25 μm) parabiotic pairings. FIG. 1D shows quantification of average neurite length from young isochronic and heterochronic parabionts. The length of the longest visible neurite was measured in 250 neurons (measured in random fields across 5 sections per mouse). FIG. 1E shows quantification of average neurite length from old isochronic and heterochronic parabionts as described for young mice. Mean+SEM; *, P<0.05; **, P<0.01 t-test.

FIGS. 2A-2E show that exposure of a young adult brain to an old systemic environment decreases synaptic plasticity and impairs spatial learning and memory. FIG. 2A shows quantification of neurogenesis in the young DG after plasma injection. Data are from 7-8 mice per group (5-7 sections per mouse). FIGS. 2B and 2C show experiments where synaptic plasticity of young isochronic and heterochronic parabionts was examined after five weeks of parabiotic pairing in hippocampal slices by extracellular electrophysiological recordings using a long-term potentiation (LTP) paradigm. FIG. 2B shows representative electrophysiological profiles collected from individual young (3 months) isochronic and heterochronic parabionts during LTP recordings from the DG. FIG. 2C shows that LTP levels recorded from the DG were lower in the hippocampus of young heterochronic (100.6±34.3%) versus young isochronic (168.5±15.8%) parabionts following 40 minutes after induction. Data are from 4-5 mice per group. FIGS. 2D and 2E show how spatial learning and memory was assessed using the radial arm water maze (RAWM) paradigm in young (3 months) adult male mice injected intravenously with plasma isolated from young (3-4 months) and old (18-20 months) mice every three days for 24 days. FIG. 2D shows a schematic of the RAWM paradigm. The goal arm location containing the platform remains constant, while the start arm is changed during each trial. On day one during the training phase, mice are trained for 15 trials, with trials alternating between visible (white) and hidden (shaded) platform. On day two during the testing phase, mice are tested for 15 trials with the hidden (shaded) platform. Entry into an incorrect arm is scored as an error, and errors are averaged over training blocks (three consecutive trials). FIG. 2E shows how learning and memory deficits were quantified as the number of entry arm errors made prior to finding the target platform. Data are from 7-8 mice per group. Mean±SEM; *, P<0.05; **, P<0.01; t-test (2A), ANOVA, Tukey's post-hoc test (2E).

FIGS. 3A-3I show that systemic chemokine levels increase during normal aging and heterochronic parabiosis and correlate with the age-dependent decrease in neurogenesis. FIG. 3A shows a Venn diagram outlining the results from the normal aging and parabiosis proteomic screens. The seventeen blood borne factors whose levels increased with aging and correlated strongest with the age-related decline in neurogenesis are shown in left side circle, the fourteen blood borne factors that increased between young isochronic and young heterochronic parabionts are shown in right side circle, and the five factors elevated in both screens are shown in the intersection in light grey area. (5-6 animals per age group were used) FIGS. 3B-3E show changes in plasma concentrations for CCL2 (3B, 3D) and CCL11 (3C, 3E) with age (3B, 3C) and from an independent proteomic screen in young heterochronic parabionts pre- and post-parabiotic pairing (3D, 3E). FIGS. 3F-3I show changes in concentrations for CCL2 (3F, 3H) and CCL11 (3G, 3I) in healthy, cognitively normal human subjects in plasma with age (3F, 3G) and in CSF between young (20-45 years) and old (65-90 years) (3H, 3I). Dot plots with mean; *, P<0.05; **, P<0.01; ***, P<0.001 t-test (c,d), ANOVA, Tukey's post-hoc test (3A, 3B), and Mann-Whitney U Test (3H, 3I).

FIGS. 4A-4G show that systemic exposure to the age-related chemokine CCL11 inhibits neurogenesis and impairs spatial learning and memory in young adult animals. FIG. 4A shows an experiment where Dcx-luc reporter mice (2-3 months) were injected with either recombinant murine CCL11 or PBS (vehicle) every other day for four days (7 mice per group). Bioluminescence was recorded in living mice at days zero and four, and representative images are shown for each treatment group. FIG. 4B shows results when bioluminescence was quantified as photons/s/cm2/steridan and differences expressed as changes in fold-induction between day zero and four. FIG. 4C shows quantification of neurogenesis in the DG after systemic drug administration after an independent cohort of 3-month-old wild type male mice was injected intraperitoneally with recombinant murine CCL11 or vehicle alone, and in combination with an anti-CCL11 neutralizing antibody or an isotype control antibody four times over ten days (6-10 mice per group). FIG. 4D shows quantification of the relative number of BrdU and NeuN double positive cells compared to the total number of BrdU positive cells in the DG mice that were systemically administered with either recombinant murine CCL11 or vehicle alone from the group above were injected with BrdU daily for three days prior to sacrifice. FIGS. 4E-4F show quantification of neurogenesis in the DG after systemic and stereotaxic drug administration. Data are from 3-10 young adult mice (2-3 months) per group (5 sections per mouse) after young adult mice were given unilateral stereotaxic injections of either anti-CCL11 neutralizing antibody or an isotype control antibody followed by systemic injections with either recombinant CCL11 or PBS. FIG. 4G shows how spatial learning and memory was assessed using the RAWM paradigm in young adult male mice (3 months) injected with recombinant murine CCL11 or PBS (vehicle) every three days for five weeks. Cognitive deficits were quantified as the number of entry arm errors made prior to finding the target platform. All the histological and behavioral assessments were carried out by investigators blinded to the treatment of the mice. Data is represented as Mean±SEM; *, P<0.05; **, P<0.01; t-test (4B, 4D, 4E, 4F), ANOVA, Dunnet's or Tukey's post-hoc test (4C, 4G).

FIGS. 5A-5D show that adult neurogenesis decreases as neuroinflammation increases in the DG during aging. We performed an immunohistochemical detection of newly differentiated Doublecortin (Dcx)-positive neurons, long-term BrdU-retaining cells (arrowheads), CD68-positive activated microglia, and GFAP-positive astrocytes in the DG of the hippocampus from adult mice at 6 and 18 months of age. FIGS. 5A-5D show quantification of age-related cellular changes in the adult DG. Data are from 5-10 mice per age group (5-7 sections per mouse), each dot represents the mean number per mouse Animals were given 6 days of BrdU injections and euthanized 21 days following the last injection. FIG. 5C shows age-related increase of relative immunoreactivity to CD68, a marker for microglia activation. FIG. 5D shows that GFAP reactivity did not significantly change with age. Dot plots with mean; ***, P<0.001, ANOVA, Dunnet's post-hoc test.

FIGS. 6A-6B show that synaptic plasticity and cognitive function are impaired in the hippocampus of old versus young animals. In FIG. 6A synaptic plasticity of normal aging animals was examined in hippocampal slices by extracellular electrophysiological recordings using a long-term potentiation (LTP) paradigm. LTP levels recorded from the DG were lower in the hippocampus of old (100.25±14.0%, n=7) versus young (201.1±40.6%, n=6) animals following 40 minutes after induction. FIG. 6B shows how spatial learning and memory was assessed during normal aging in young (2-3 months) versus old (18-20 months) adult animals (7-8 C57Bl/6 male mice per group). Old mice demonstrate impaired learning and memory for platform location during the testing phase of the task. Cognitive deficits were quantified as the number of entry arm errors made prior to finding the target platform. All data is represented as Mean±SEM; *, P<0.05; **, P<0.01; ANOVA, Tukey's post-hoc test.

FIGS. 7A-7F show that heterochronic parabiosis reduces proliferation and progenitor frequency in the DG of young animals while increasing proliferation in aged animals. After five weeks of parabiosis, animals were injected with BrdU for three days prior to sacrifice. BrdU immunostaining was performed for young (3-4 months) and aged (18-20 months) isochronic and heterochronic parabionts. FIG. 7A shows quantification of proliferation in the young DG after parabiosis. Data are from 8 mice for isochronic and 6 mice for heterochronic groups. FIG. 7B shows quantification of proliferation in the aged DG after parabiosis. Data are from 4 mice for isochronic and 6 mice for heterochronic groups. Sox2 immunostaining was also performed for young (3-4 months) isochronic and heterochronic parabionts. FIG. 7C shows quantification of Sox2-positive progenitor cells in the young DG after parabiosis. Data are from 8 mice for isochronic and 6 mice for heterochronic groups. FIGS. 7D and 7E show quantification of neurogenesis (Dcx, Doublecortin-positive cells) in the DG during normal aging and after isochronic (Iso) or heterochronic (Het) parabiosis. 7A data are from 10 normal aged (18 months old) mice, 6 isochronic parabionts (18-20 months old) and 12 heterochronic parabionts (18-20 months old). 7F shows quantification of neurite length during normal aging and after parabiosis in Dcx-positive cells. Dendritic length remained unchanged between unpaired normal aged animals and isochronic parabiotic animals. All data are from 5-7 sections per mouse; bars are mean±SEM; * P<0.05; ** P<0.01; n.s., not significant; t-test.

FIGS. 8A-8E show that circulatory system is shared between animals during parabiosis. FIGS. 8A-8D show a subset of four parabiotic pairs were generated by joining young (2-3 months old) actin-GFP transgenic with young (2-3 months old) and aged (18 months old) non-transgenic mice. Blood was isolated two weeks after surgery and flow cytometric analysis was done on fixed and permeabilized blood cells. Representative flow-cytometry plots demonstrate the frequency of GFP-positive cells in a GFP-transgenic (tg) parabiont (a,c) and wild-type (wt) parabiont (8B, 8D) at the time of sacrifice. MFI, mean fluorescence intensity. FIG. 8E shows quantification of GFP-positive cells in the DG of the hippocampus in young and aged wild-type parabionts after parabiosis with young actin-GFP-positive parabionts. 5 sections per mouse; bars are mean±SEM; n.s., not significant; t-test.

FIGS. 9A-9C show that changes in concentrations of selected secreted plasma proteins correlate with declining neurogenesis in aging and heterochronic parabiosis. FIG. 9A shows an analysis of plasma protein correlations with decreased neurogenesis in the aging mouse samples using the Significance Analysis of Microarray software (SAM 3.00 algorithm). SAM assigns d-scores to each gene or protein on the basis of a multi-comparison analysis of expression changes and indicates significance by q-value. FIG. 9B shows unsupervised clustering of secreted signaling factors that were significantly associated with age-related decreased neurogenesis with a false discovery rate of 7.34% or less (SAM, q 7.34). Mouse age groups are indicated at the top of the node map as boxes in which youngest ages are tan and oldest ages are red. Thus cluster analysis of systemic factors associated with decreased neurogenesis also produce a reasonable separation of samples by age. Color shades in the node map indicate higher (purple) or lower (green) relative plasma concentrations. FIG. 9C shows quantitative fold changes in soluble signaling factors between isochronic versus heterochronic parabiotic groups. Color shades indicate increases (darker gray scale) and decreases (lighter grey scale) in relative plasma concentrations (mean±SEM of fold changes observed with parabiosis; n.c. denotes no significant change).

FIGS. 10A-C show that systemic administration of CCL11 reduces cell proliferation but not glial differentiation in the DG of young animals. Young adult male mice (2-3 months old) were injected with either recombinant murine CCL11 or PBS (vehicle) through intraperitoneal injections every three days for ten days for a total of four injections Animals were injected with BrdU for three days prior to sacrifice. FIG. 10A shows that a significant increase above basal CCL11 plasma levels was measured in mice treated systemically with recombinant CCL11, but no relative change was observed in animals receiving PBS. Blood was collected by mandibular vein bleed prior to systemic drug administration and by intracardial bleed at time of sacrifice using EDTA as an anticoagulant. Plasma was generated by centrifugation of blood. Samples were diluted 1:10 and CCL11 was detected by Quantikine ELISA following the manufacturer's manual (R&D Systems). BrdU immunostaining was performed in the DG for each treatment group. FIG. 10B shows quantification of BrdU-positive cells in the DG after systemic drug administration. Data are from 5-10 mice per group (5 sections per mouse). Confocal microscopy images from the subgranular zone of the DG of brain sections immunostained for BrdU in combination with GFAP was also performed for both treatment groups. FIG. 10C shows quantification of the relative number of BrdU and GFAP double positive cells out of all BrdU-positive cells in the DG after systemic CCL11 administration. Data are from 5 mice per group (3 sections per mouse). Bars show mean±SEM; *, P<0.05; **, P<0.01; n.s., not significant; t-test (10C) or ANOVA, Dunnet's post-hoc test (10A, 10B).

FIGS. 11A-11C show that systemic administration of MCSF does not alter neurogenesis in the DG of young animals. FIGS. 11A and 11B show a comparison of plasma concentrations for MCSF in normal aged (6, 12, 18 and 24 months old) (11A) and young heterochronic parabionts pre and post parabiotic pairing (11B). Young adult male mice (2-3 months old) were injected with either recombinant MCSF alone or PBS as a vehicle control through intraperitoneal injections every three days for ten days. Neurogenesis was analyzed by immunostaining for Dcx. FIG. 11C shows quantification of neurogenesis in the DG after systemic drug administration. Data are from 5 mice per group (5 sections per mouse). Bars show mean±SEM; n.s, not significant; t-test (11B and 11C) or ANOVA, Dunnet's post-hoc test (11A).

FIGS. 12A-12H show that age-related blood borne factors, including CCL11 and CCL2, inhibit NPC function and neural differentiation in vitro. FIG. 12 A shows an experiment where primary NPCs were exposed to serum isolated from young (2-3 months) or old (18-22 months) mice for four days in culture under self-renewal conditions. The number of neurospheres formed in the presence of old serum was decreased compared to neurospheres formed in the presence of young serum. FIG. 12 B shows a dose-dependent decrease in the number of neurospheres formed from primary mouse NPCs after exposure to murine recombinant CCL11 for four days in culture under self-renewal conditions. FIG. 12C shows decrease in neurosphere formation after exposure to murine recombinant CCL11 compared with PBS (vehicle) control is rescued by addition of anti-CCL11 neutralizing antibody but not by a non-specific isotype control antibody. FIG. 12D shows a decrease in the number of neurospheres formed from primary mouse NPCs after exposure to murine recombinant CCL2 is rescued by addition of anti-CCL2 neutralizing antibody. FIG. 12F shows a quantification of decreased neurosphere size after exposure to CCL11. FIG. 12G shows a quantification of decreased neuronal differentiation as a function of reduced expression of Dcx promoter-controlled eGFP in stably transfected human derived NTERA cells after exposure to human recombinant CCL11 (12G) or CCL2 (12H), compared with PBS (vehicle) as a control. FIG. 12G shows that decreased neuronal differentiation is rescued by addition of anti-CCL11 neutralizing antibody but not by a non-specific isotype control antibody. FIG. 12H shows quantification of dose dependent decrease in neuronal differentiation after exposure to human recombinant CCL2. Human NTERA-EGFP reporter cells were cultured under differentiation conditions (RA, retinoic acid) for 12 days and relative Dxc reporter gene activity was measured as fluorescence intensity. In vitro data are representative of three independent experiments done in triplicate. Bars are mean±SEM; *, P<0.05; **, P<0.01; ***, P<0.001; t-test (a,f) or ANOVA, Dunnet's post-hoc test (12B-12D, 12G, 12H).

FIG. 13 shows that neurogenesis is inhibited by direct exposure to CCL11 in vivo. Young adult mice were injected stereotaxically with either recombinant CCL11 or PBS into the left or right DG. Dcx-positive cells in adjacent sides of the DG within the same section were shown for treatment groups. Quantification of neurogenesis in the DG after stereotactic CCL11 administration is shown. All data are from 4-5 young adult mice (2-3 months of age) per group (5 sections per mouse). Bars show mean±SEM; *, P<0.05; t-test

FIGS. 14A-14B show a proposed model illustrating the cellular and functional impact of age-related systemic molecular changes on the adult neurogenic niche. Schematic of cellular changes occurring in the neurogenic niche during normal aging and heterochronic parabiosis. Levels of blood-borne factors, including the chemokines CCL11 and CCL2, increase during normal aging and heterochronic parabiosis. These systemic changes contribute to the decline in neurogenesis observed in the adult brain and functionally impair synaptic plasticity and learning and memory. Cellular impact illustration is provided in FIG. 14A and functional impact scenario is provided in FIG. 14B. Cell types illustrated include neural stem cells (NPC), neurons, astrocytes, and microglia (FIG. 14A).

### DETAILED DESCRIPTION

The present application provides methods of diagnosis, prognosis and monitoring of age-related diseases using biomarkers that have been linked to biological aging process. The methods are, at least in part, based on a discovery that altered expression patterns of certain biological markers are associated with biological aging processes. These markers comprise at least Eotaxin/CCL11, β2-microglobulin, MCP-1 and Haptoglobulin, increased expression of which has been shown to be associated with increase in biological aging process.

It should be understood that this invention is not limited to the particular methodology, protocols, and reagents, etc., described herein and as such may vary. The terminology used herein is for the purpose of describing particular embodiments only, and is not intended to limit the scope of the present invention, which is defined solely by the claims.

As used herein and in the claims, the singular forms include plural references and vice versa unless the context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Other than in the operating examples, or where otherwise indicated, all numbers expressing quantities of ingredients or reaction conditions used herein should be understood as modified in all instances by the term “about.”

All patents and other publications identified in the specification, figures and examples are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

Unless defined otherwise, all technical and scientific terms used herein have the same meaning as those commonly understood to one of ordinary skill in the art to which this invention pertains. Although any known methods, devices, and materials may be used in the practice or testing of the invention, the methods, devices, and materials in this regard are described herein.

### INTRODUCTION

Diminished tissue regeneration is one of the hallmarks of aging. The regenerative properties of most tissues gradually decline with age, mainly due to the age-associated declined activity of tissue-specific stem cells. Age-associated diminished tissue regeneration happens in most organs, for example, aging brain has a high incidence of age-dependent degeneration, propensity for age-related diseases, and a low tissue regenerative potential. As in other organ systems, stem cell activity in the brain declines with age.

One of the objectives is to understand how age-related changes in systemic biomarkers, such as β2-Microglobulin (β2M), regulate the decline in stem cell function, such as neural stem cell or progenitor cell (NPC) function, observed during aging. Embodiments of the present invention provides for insights into the molecular mechanisms responsible for tissue aging in organisms, such as central nervous system (CNS), to understand and prevent age-associated disorders or diseases, such as age-dependent tissue degeneration and neurodegenerative diseases. Stem cells have been studied due to their potential for mediating enhanced tissue repair, regeneration from degenerative diseases, and amelioration of normal organ dysfunction attributed to the aging process. However, it is unclear how the aging process modulates tissue-specific stem cell activity, and if such modulation results in the inability of stem cells to maintain both the structure and function of organs within an organism during aging. Studying the possibility and process of harnessing stem cells to reverse normal aging may help clarify these questions. In this regard, in some embodiments, the effect of aging on NPC function in the CNS are studied to investigate the associated onset of cognitive impairments and lack of neural repair in response to neurodegenerative diseases such as Alzheimer's disease[4].

The present invention is based, at least in part, on the following experimental data.

During aging both regenerative capacity and cognitive function dramatically deteriorate in the adult brain (Rando, T. A., Nature 441 (7097), 1080-1086 (2006); Rapp, P. R. & Heindel, W. C., Curr Opin Neurol 7 (4), 294-298 (1994)). Interestingly, associated stem cell and cognitive impairments can be ameliorated through systemic perturbations such as exercise (van Praag, H., Shubert, T., Zhao, C., & Gage, F. H., J Neurosci 25 (38), 8680-8685 (2005)). Here, using heterochronic parabiosis we show that blood-borne factors present in the systemic milieu can inhibit or rejuvenate adult neurogenesis in an age dependent fashion in mice. Accordingly, exposing a young animal to an old systemic environment, or to plasma from old mice, decreased synaptic plasticity and impaired spatial learning and memory. We identify chemokines—including CCL2/MCP-1 and CCL11/Eotaxin—whose plasma levels correlate with reduced neurogenesis in aged mice, and whose levels are increased in plasma and cerebral spinal fluid of healthy aging humans. Finally, increasing peripheral chemokine levels in vivo in young mice decreased adult neurogenesis and impaired spatial learning and memory. Together our data indicate that the decline in neurogenesis, and cognitive impairments, observed during aging can be in part attributed to changes in blood-borne factors.

Stem cell activity decreases dramatically with age in tissues including the brain (Rando, T. A., Nature 441 (7097), 1080-1086 (2006)). In the central nervous system (CNS), aging results in a decline in adult neural stem/progenitor cells (NPCs) and neurogenesis, with concomitant impairments in cognitive functions (van Praag, H., Shubert, T., Zhao, C., & Gage, F. H., J Neurosci 25 (38), 8680-8685 (2005); Clelland, C. D. et al., Science 325 (5937), 210-213 (2009)). Adult neurogenesis occurs in local microenvironments, or neurogenic niches, in the subventricular zone (SVZ) of the lateral ventricles and the subgranular zone (SGZ) of the hippocampus (Gage, F. H., Science 287 (5457), 1433-1438 (2000); Alvarez-Buylla, A. & Lim, D. A., Neuron 41 (5), 683-686 (2004)). Permissive cues within the neurogenic niche are thought to drive the production of new neurons and their subsequent integration into the neurocircuitry of the brain (Zhao, C., Deng, W., & Gage, F. H., Cell 132 (4), 645-660 (2008); van Praag, H. et al., Nature 415 (6875), 1030-1034 (2002)), which directly contributes to cognitive processes including learning and memory (Clelland, C. D. et al., Science 325 (5937), 210-213 (2009); Deng, W., Aimone, J. B., & Gage, F. H., Nat Rev Neurosci 11 (5), 339-350; Zhang, C. L., Zou, Y., He, W., Gage, F. H., & Evans, R. M., Nature 451 (7181), 1004-1007 (2008)). Importantly, the neurogenic niche is localized around blood vessels (Shen, Q. et al., Science 304 (5675), 1338-1340 (2004); Carpentier, P. A. & Palmer, T. D., Immune influence on adult neural stem cell regulation and function. Neuron 64 (1), 79-92 (2009)) that lack a classical blood-brain-barrier (BBB) (Shen, Q. et al., Cell Stem Cell 3 (3), 289-300 (2008); Tavazoie, M. et al., Cell Stem Cell 3 (3), 279-288 (2008); Currle, D. S. & Gilbertson, R. J., Cell Stem Cell 3 (3), 234-236 (2008), allowing for potential communication with the systemic environment. Therefore, the possibility arises that diminished adult neurogenesis during aging may be modulated by the balance of two independent forces—intrinsic CNS-derived cues previously reported (Renault, V. M. et al., Cell Stem Cell 5 (5), 527-539 (2009); Molofsky, A. V. et al., Nature 443 (7110), 448-452 (2006); Lie, D.C. et al., Nature 437 (7063), 1370-1375 (2005)), and cues extrinsic to the CNS delivered by blood. We hypothesized that age-related systemic molecular changes could cause a decline in neurogenesis and impair cognitive function during aging.

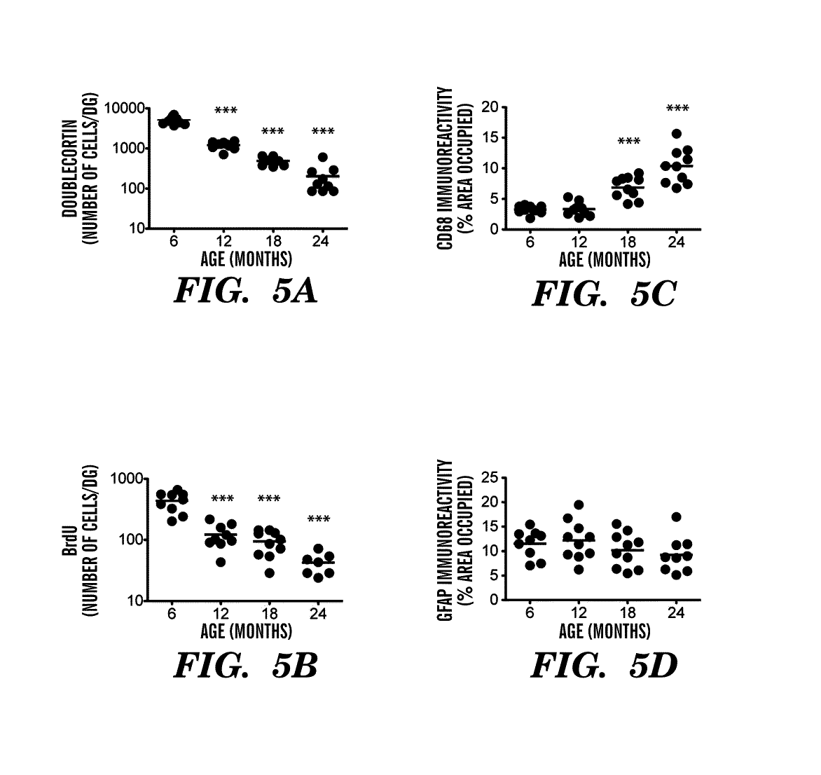

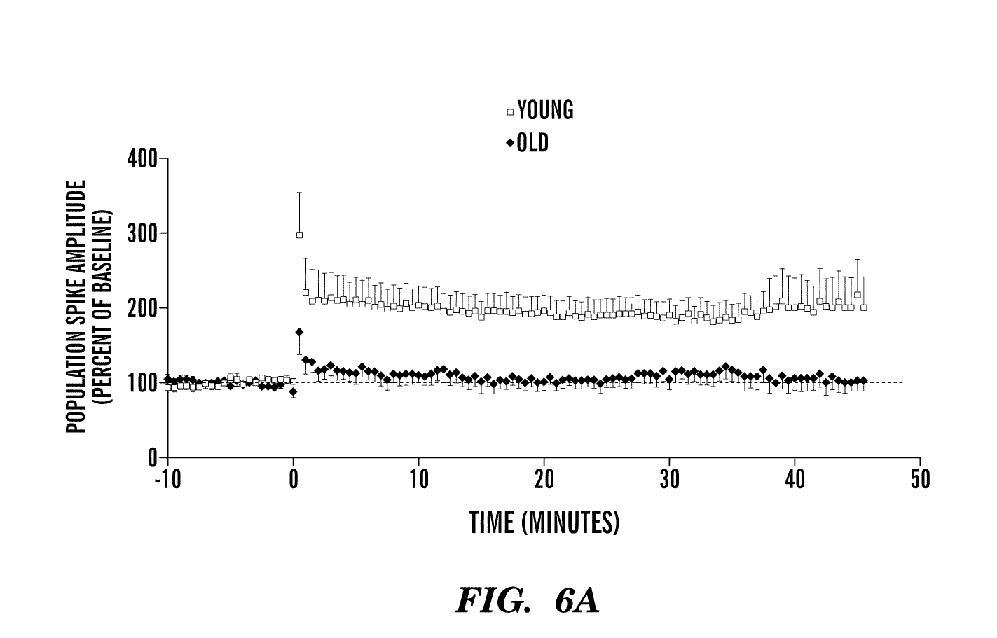

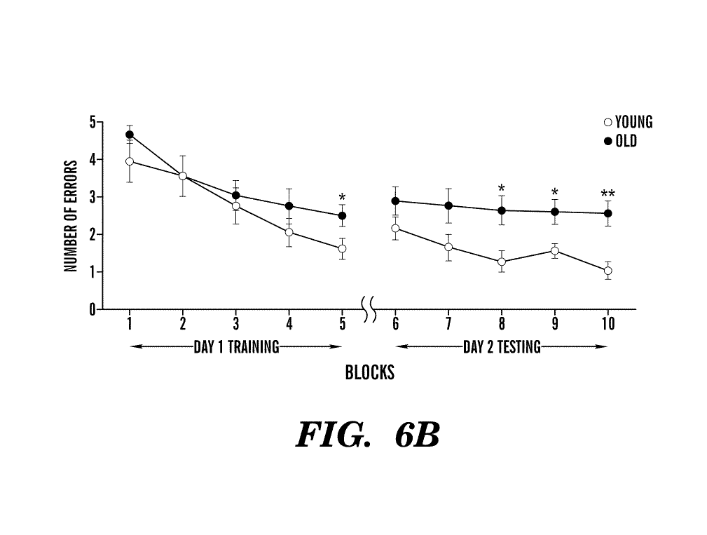

We first characterized the aging neurogenic niche by assessing cellular changes in newly differentiated neurons, neural progenitors, microglia, and astrocytes in the dentate gyrus (DG) of the hippocampus in mice at 6, 12, 18 and 24 months of age (FIG. 5A-5D), and observed changes consistent with a dramatic decrease in adult neurogenesis (van Praag, H., Shubert, T., Zhao, C., & Gage, F. H., J Neurosci 25 (38), 8680-8685 (2005)) and a concomitant increase in neuroinflammation with age (Lucin, K. M. & Wyss-Coray, T., Neuron 64 (1), 110-122 (2009)). Additionally, we used a long-term potentiation (LTP) paradigm to examine synaptic plasticity, and detected lower LTP levels from the DG of old (18 months) versus young (3 months) animals (FIG. 6A). Lastly, we assessed hippocampal dependent spatial learning and memory using the radial arm water maze (RAWM) paradigm (Alamed, J., Wilcock, D. M., Diamond, D. M., Gordon, M. N., & Morgan, D., Two-day radial-arm water maze learning and memory task; robust resolution of amyloid-related memory deficits in transgenic mice. Nat Protoc 1 (4), 1671-1679 (2006)). During the training phase all animals showed learning capacity for the task (FIG. 6B). However, old mice demonstrated impaired learning and memory for platform location compared to young mice during the testing phase of the task (FIG. 6B), consistent with a decrease in cognitive function during normal aging (Rapp, P. R. & Heindel, W. C., Curr Opin Neurol 7 (4), 294-298 (1994)).

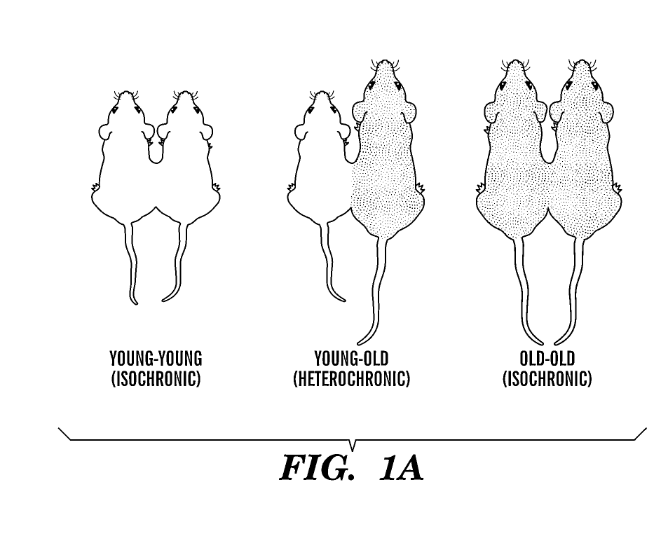

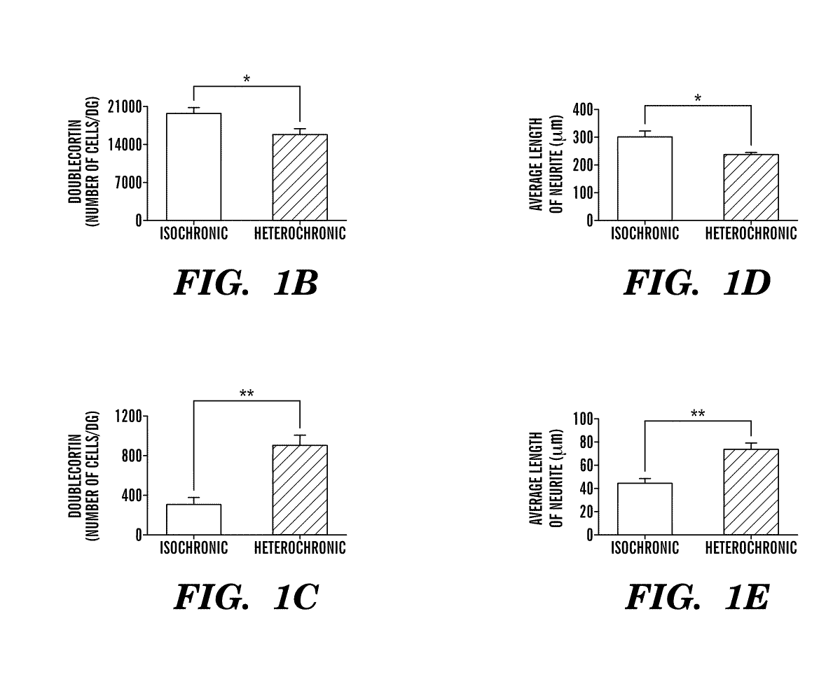

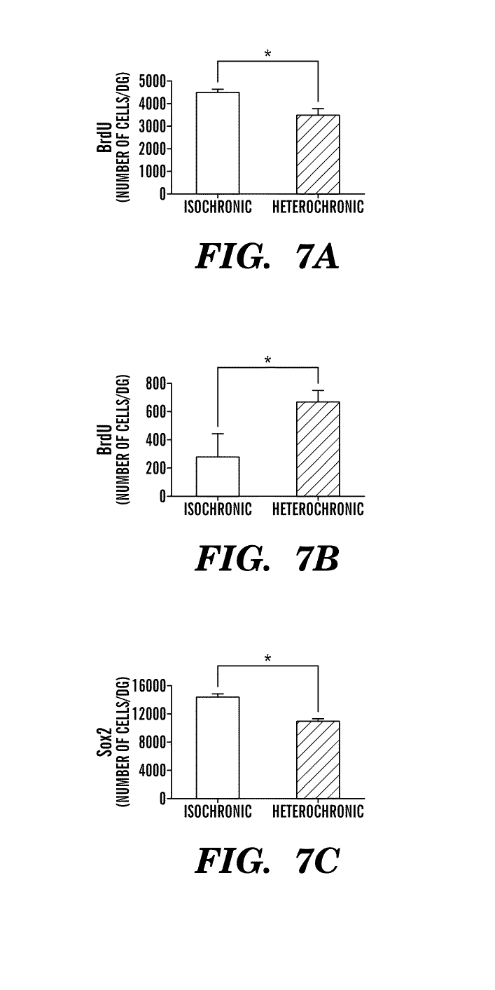

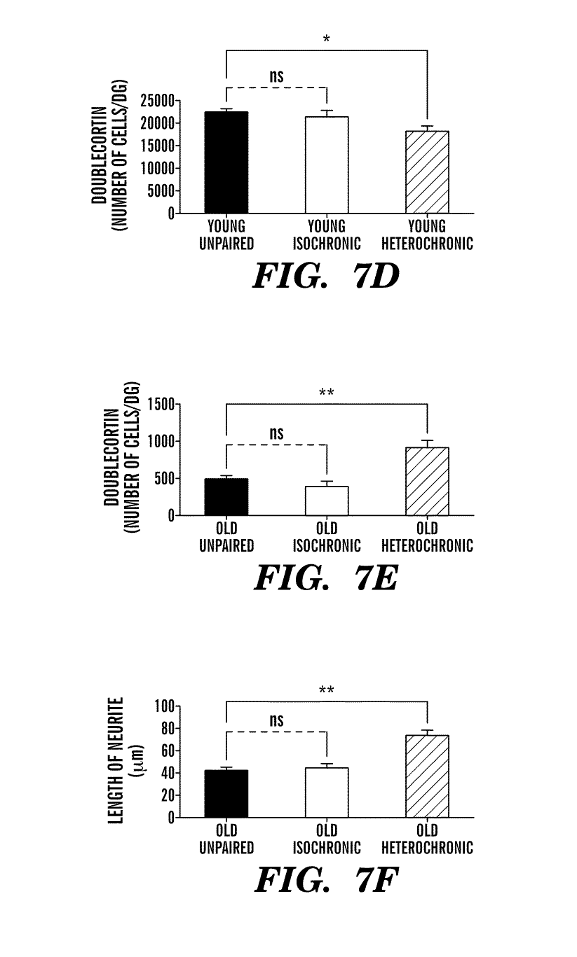

To determine whether peripheral systemic factors contributed to the decline in neurogenesis with age we utilized a model of parabiosis. Specifically, neurogenesis in the DG of the hippocampus was investigated in the setting of isochronic (young-young (3-4 months) and old-old (18-20 months)) and heterochronic (young-old) parabiotic pairings (FIG. 1A). Remarkably, the number of Doublecortin (Dcx)-positive newly born neurons in young heterochronic parabionts decreased 20% compared to young isochronic parabionts (FIG. 1B). Likewise, BrdU-positive cells (FIG. 7B) and Sox2-positive progenitors (FIG. 7C) showed a similar decrease. In contrast, we observed a 3-fold increase in the number of Dcx-positive neurons (FIG. 1C) and BrdU-positive cells (FIG. 7C) in the old heterochronic parabionts compared to isochronic old parabionts. The number of Dcx-positive neurons between unpaired age-matched animals and isochronic animals showed no significant difference, indicating that the parabiosis procedure in it of itself did not account for the observed changes (FIGS. 7D and 7E).

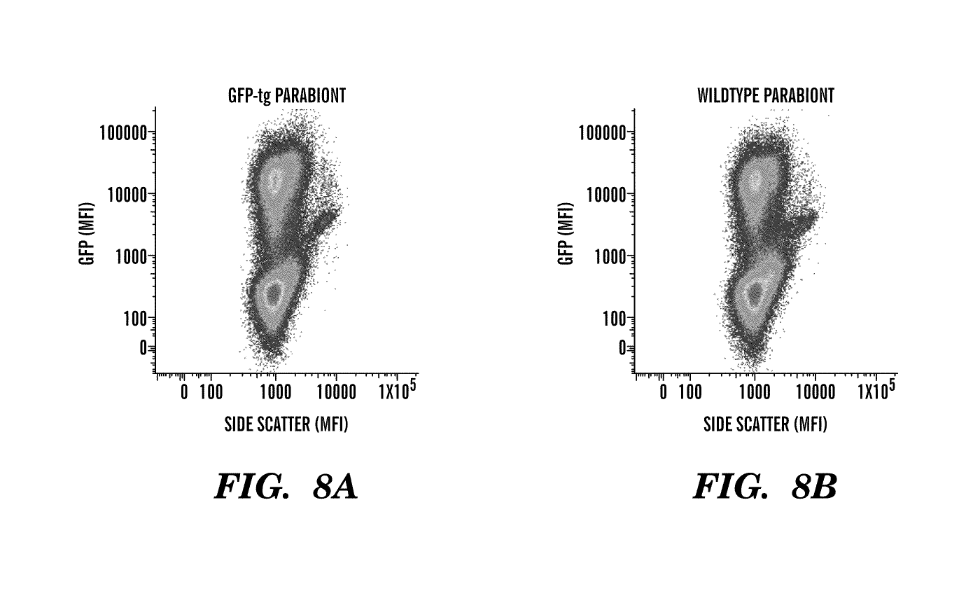

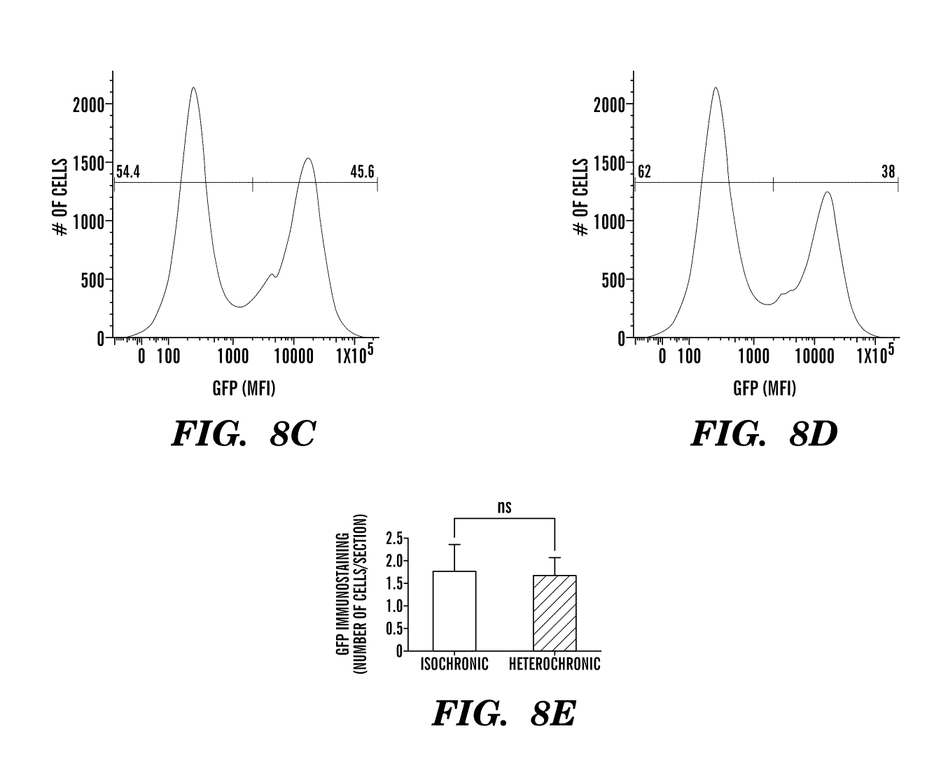

We also compared the neurite length of newly differentiated neurons in isochronic and heterochronic parabionts (FIG. 1D, 1E). Young heterochronic parabionts showed a 20% decrease in length compared to isochronic parabionts (FIG. 1D), while old heterochronic parabionts demonstrated a 40% increase in length compared to age-matched isochronic controls (FIG. 1E). Neurite length between unpaired age-matched animals and isochronic parabionts showed no significant difference (FIG. 7F). As a control, flow cytometry analysis confirmed a shared vasculature in a subset of parabiotic pairs, in which one parabiont was transgenic for green fluorescent protein (GFP, FIG. 8A-8D). Together our findings suggest that global age-dependent systemic changes can modulate neurogenesis and neurite morphology in both the young and aged neurogenic niche, potentially contributing to the decline in regenerative capacity observed in the normal aging brain.

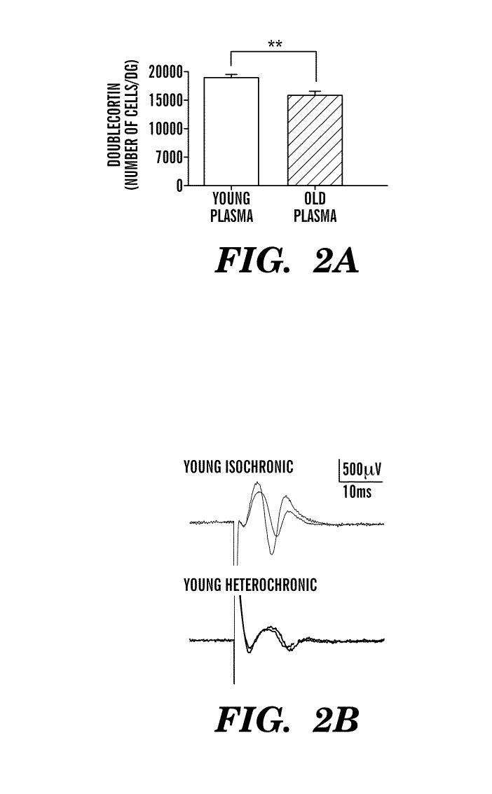

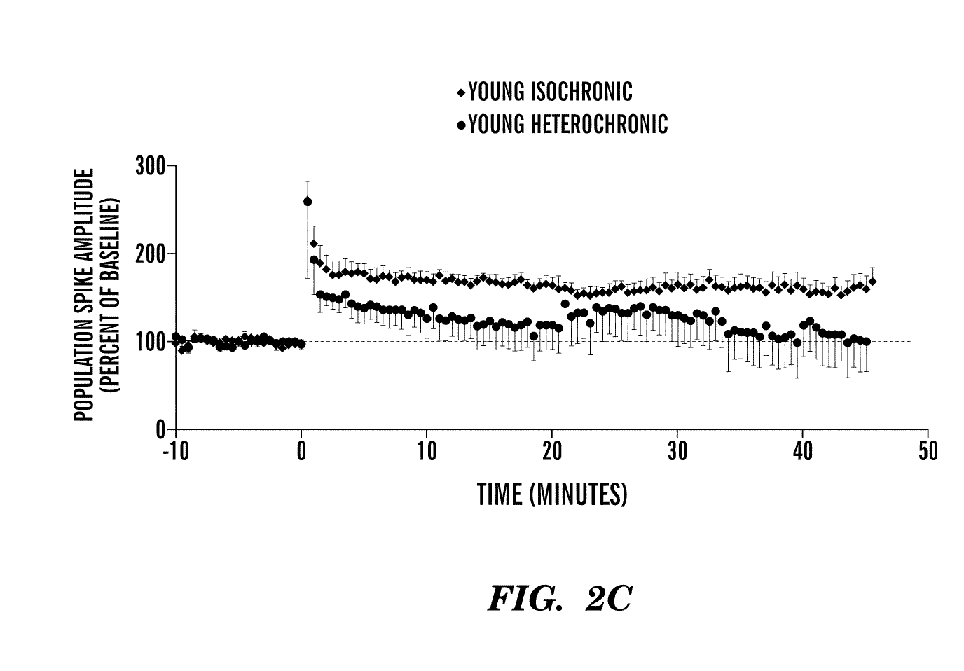

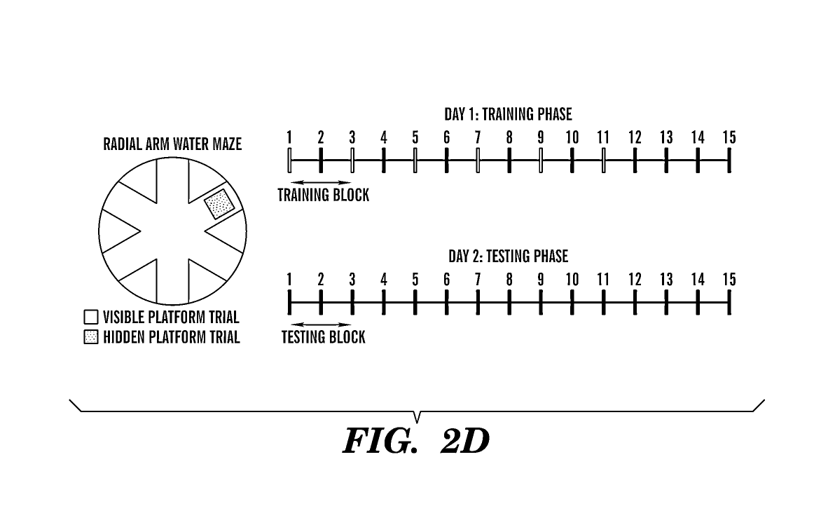

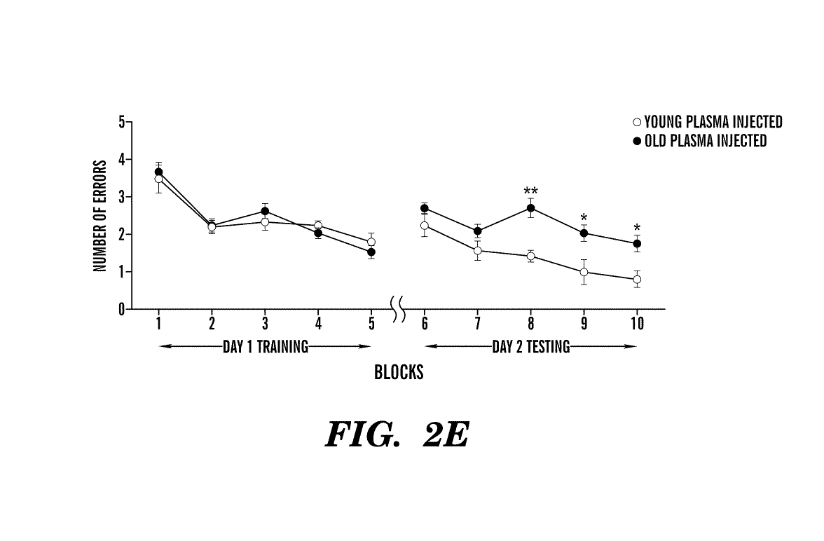

As previously reported by others (Ajami, B., Bennett, J. L., Krieger, C., Tetzlaff, W., & Rossi, F. M., Nat Neurosci 10 (12), 1538-1543 (2007)), we rarely detected peripherally derived GFP cells in the CNS of wild-type mice when joined to GFP transgenic mice, and these numbers did not differ between isochronic and heterochronic pairings (FIG. 8E), suggesting the observed effects are most likely mediated by soluble factors in plasma. To confirm that circulating factors within aged blood can contribute to reduced neurogenesis with age, we intravenously injected plasma isolated from young (3-4 months) and old (18-22 months) mice into a cohort of young adult animals. The number of Dcx-positive cells in the DG decreased in animals receiving old plasma compared to animals receiving young plasma (FIG. 2A), indicating that soluble factors present in old blood inhibit adult neurogenesis. To further investigate the functional effect of the aging systemic milieu on the young adult brain, extracellular electrophysiological recordings were done on hippocampal slices prepared from young isochronic and heterochronic parabionts (FIG. 2B). We detected a decrease in LTP levels in the medial and lateral DG of heterochronic parabionts compared to isochronic parabionts (FIG. 2C), indicating that age-related systemic changes can elicit deficits in synaptic plasticity. Lastly, given that LTP is considered a correlate of learning and memory (Bliss, T. V. & Collingridge, G. L., Nature 361 (6407), 31-39 (1993)), we sought to further evaluate the physiological effect of circulating factors present in aged blood by testing hippocampal dependent learning and memory using the RAWM paradigm in young adult mice intravenously injected with young or old plasma (FIG. 2D-2E). All mice showed similar spatial learning capacity during the training phase (FIG. 2E). However, during the testing phase animals administered with old plasma demonstrated impaired learning and memory for platform location, committing more errors in identifying the target arm compared to animals receiving young plasma (FIG. 2E). Collectively, these data indicate that factors present in aging blood inhibit adult neurogenesis, and moreover functionally contribute to impairments in synaptic plasticity and cognitive function.

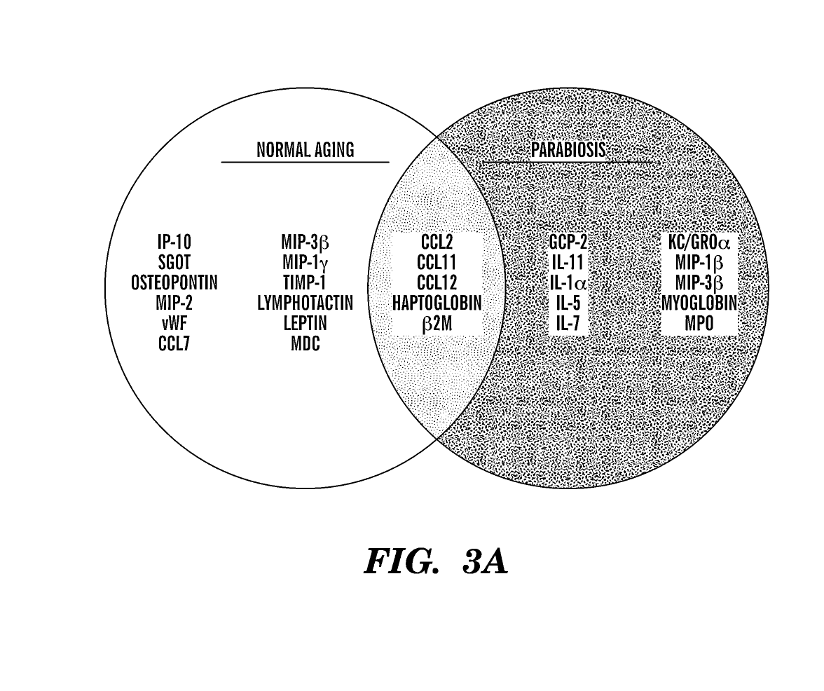

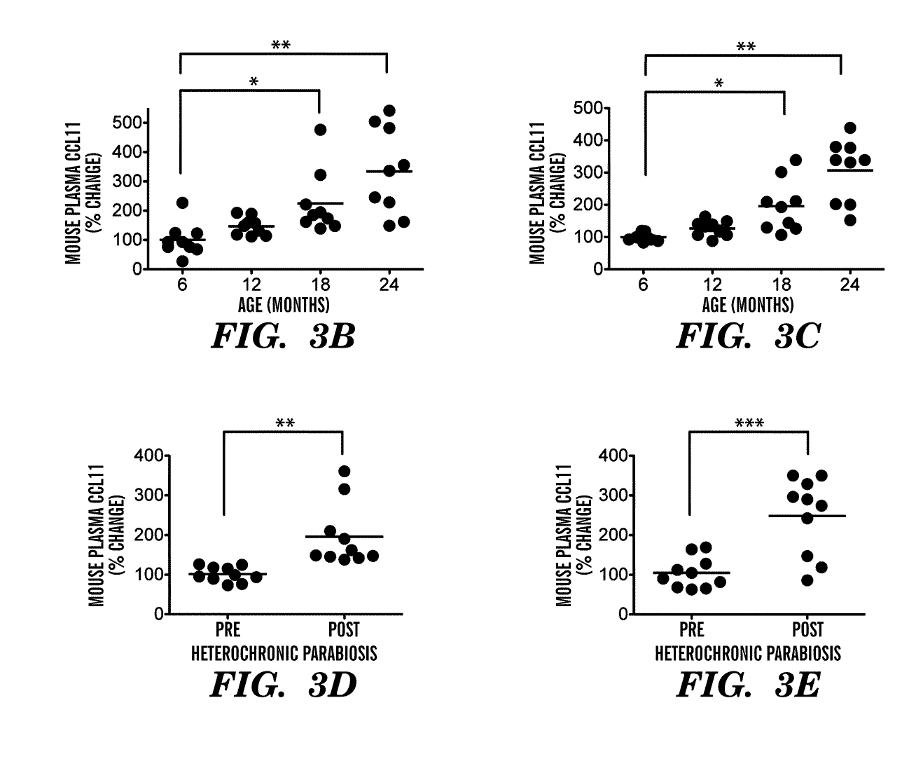

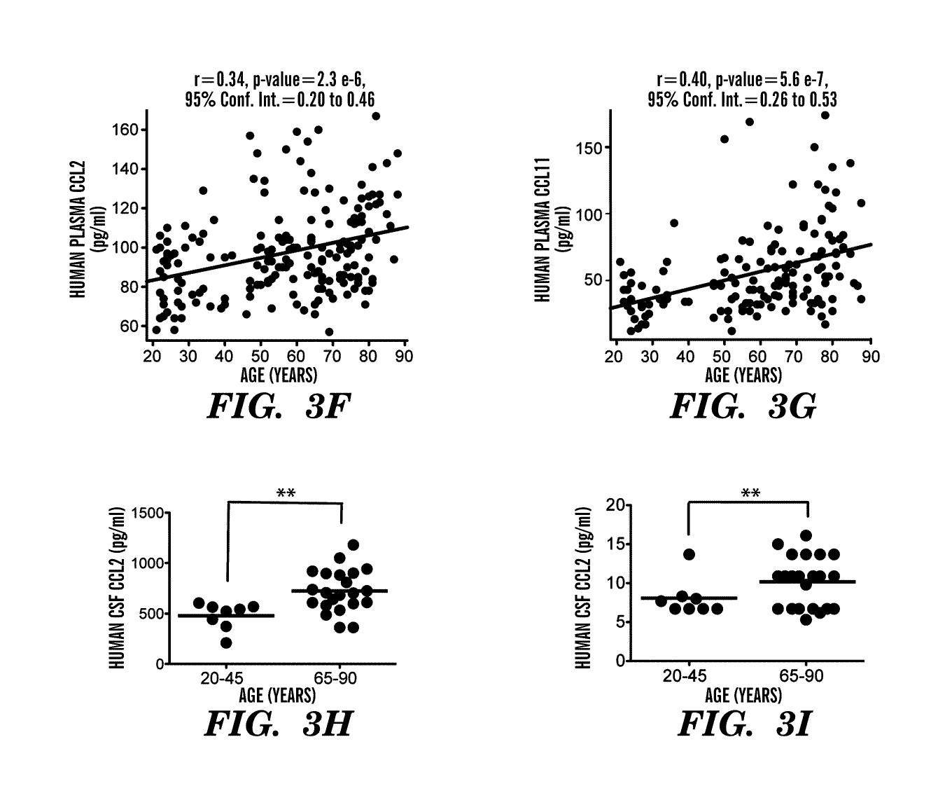

Consistent with our cellular findings in the CNS, previous studies focusing on muscle stem cells also show that exposure of the aged stem cell niche to a young systemic environment through heterochronic parabiosis results in increased regeneration after muscle injury (Conboy, I. M. et al., Nature 433 (7027), 760-764 (2005)). However, in these earlier models individual circulating factors associated with either aging and tissue degeneration, or tissue rejuvenation, have remained elusive. To identify such systemic factors, we employed a proteomic approach in which the relative levels of 66 cytokines, chemokines and other secreted signaling proteins were measured in the plasma of normal aging mice using standardized antibody-based immunoassays on microbeads (Luminex; Table 4). Using multivariate analysis, we identified seventeen blood borne proteins that correlated with the age-related decline in neurogenesis during normal aging (FIG. 3A, 9A-9B).

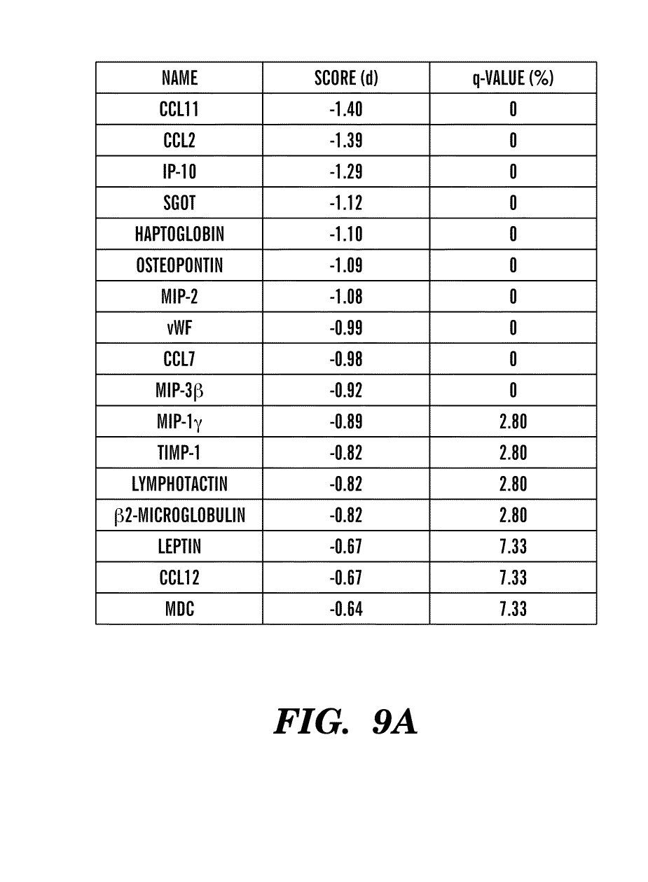

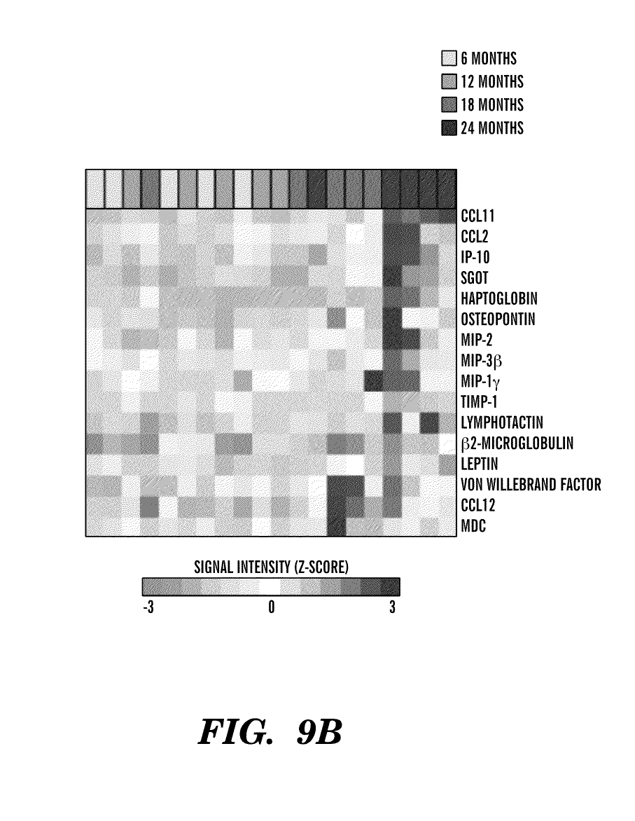

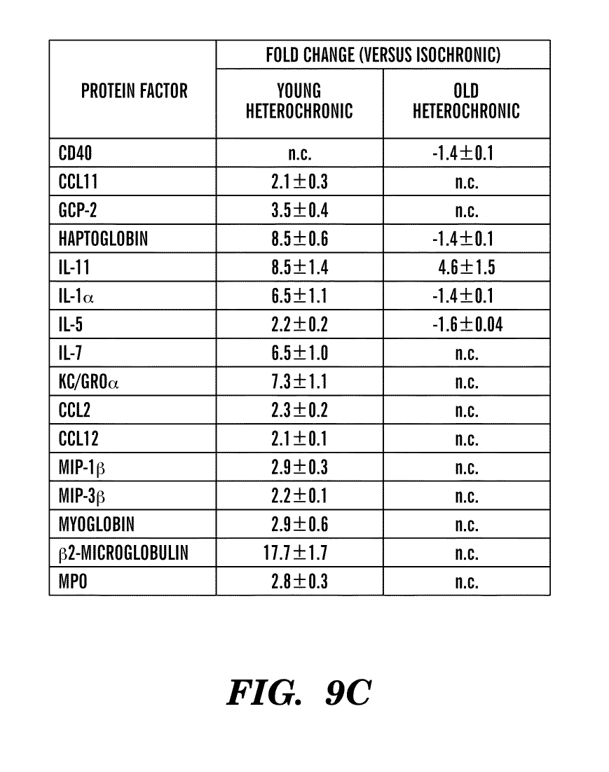

To identify systemic factors associated with heterochronic parabiosis, we analyzed plasma samples from young and old animals before and after pairings in an independent proteomic screen using the Luminex platform. Comparison of young isochronic and heterochronic cohorts identified fourteen factors with a greater than 2-fold increase in expression in the heterochronic parabionts (FIG. 3A, FIG. 9C), while comparison between old isochronic and heterochronic cohorts revealed four factors whose expression levels decreased to less than 70% of that observed in isochronic parabionts (FIG. 9C). Interestingly, only five factors—CCL2, CCL11, CCL12, β2-microglobulin and Haptoglobin—were elevated in both old unpaired and young heterochronic cohorts compared to young unpaired or isochronic cohorts (FIG. 3A). We observed a comparable increase in the relative levels of CCL2 and CCL11 in the plasma of mice during normal aging (FIG. 3B-3C) and within young mice during heterochronic parabiosis (FIG. 3D-3E).

To corroborate systemic changes in mice with changes occurring in humans, we measured CCL2 and CCL11 in archived plasma and cerebrospinal fluid (CSF) samples from healthy individuals between 20 and 90 years of age. Indeed, we detected an age-related increase in CCL2 and CCL11 measured in both plasma (3F-3G) and CSF (FIG. 3H-3I), suggesting that these age-related systemic molecular changes are conserved across species.

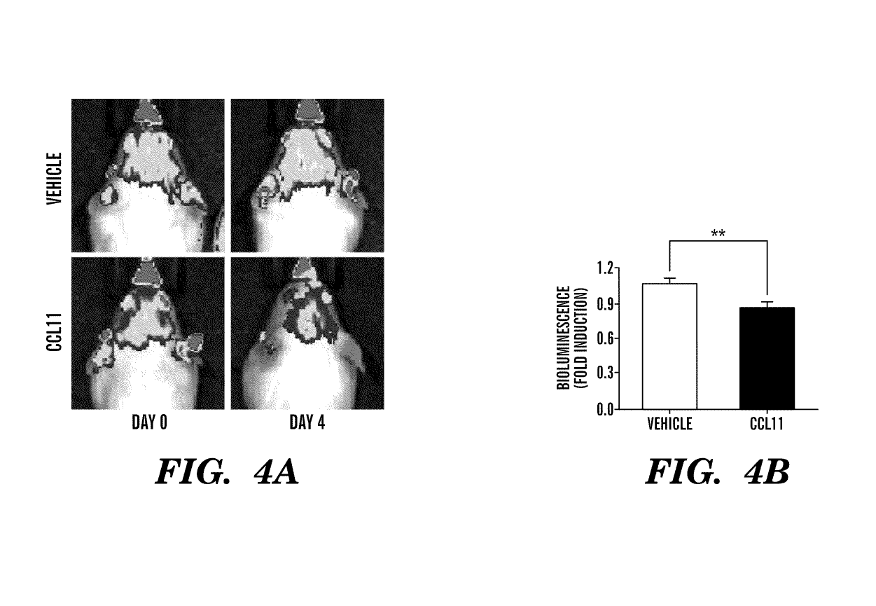

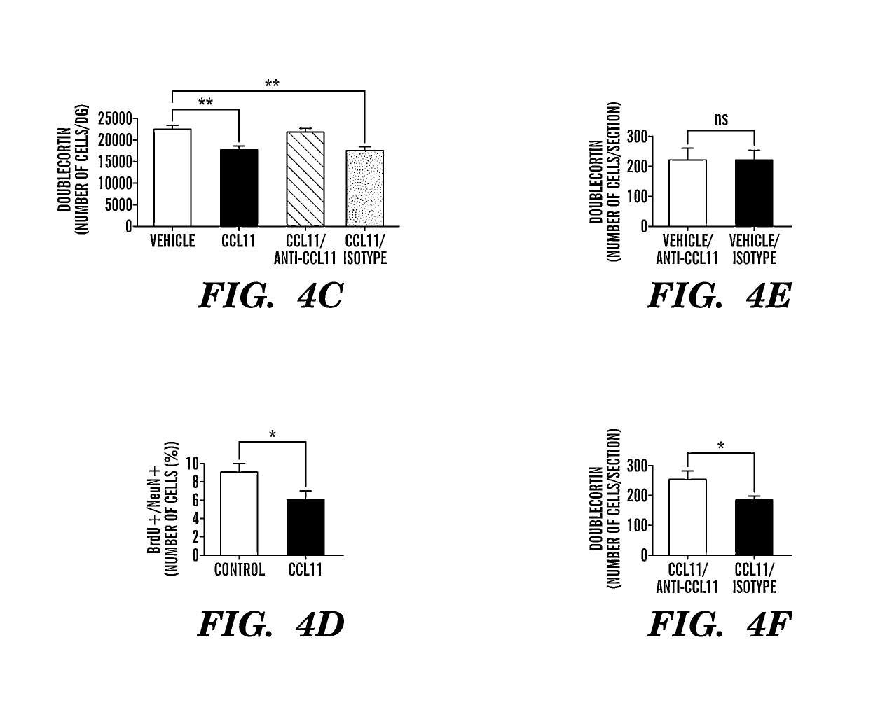

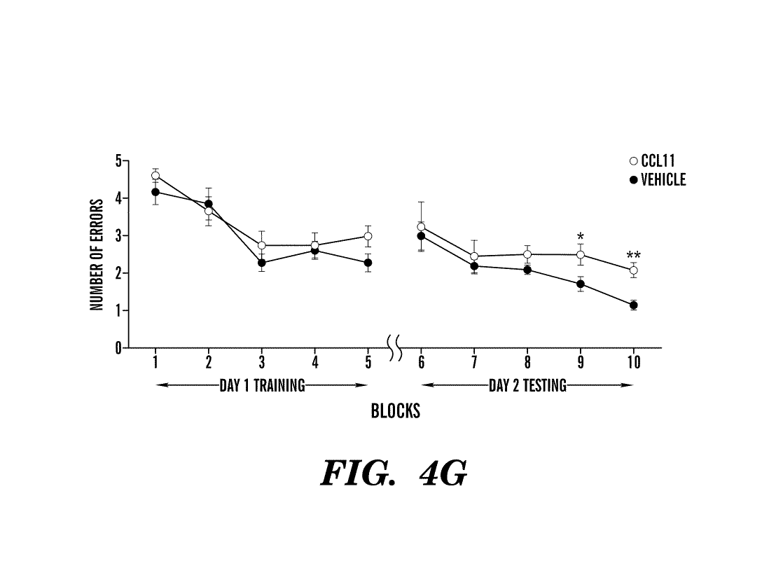

Having identified systemic factors associated with aging and decreased neurogenesis, we tested their potential biological relevance in vivo. As CCL2 had previously been linked to aging (Fumagalli, M. & d'Adda di Fagagna, F., Nat Cell Biol 11 (8), 921-923 (2009)) and shown to regulate NPC function after brain injury (Belmadani, A., Tran, P. B., Ren, D., & Miller, R. J., J Neurosci 26 (12), 3182-3191 (2006)), we decided to focus our study on CCL11, a chemokine involved in allergic responses and not previously linked to aging, neurogenesis, or cognition. We administered recombinant murine CCL11 protein through intraperitoneal injections into young adult mice and measured global changes in neurogenesis within the same mouse with a non-invasive bioluminescent imaging assay using Doublecortin-luciferase reporter mice (Couillard-Despres, S. et al., Mol Imaging 7 (1), 28-34 (2008)). This systemic administration of recombinant CCL11 caused a significant decrease in Dcx promoter-dependent luciferase activity compared with mice receiving vehicle control indicating a decrease in the number of Dcx-expressing neuroblasts (FIG. 4A-4B).

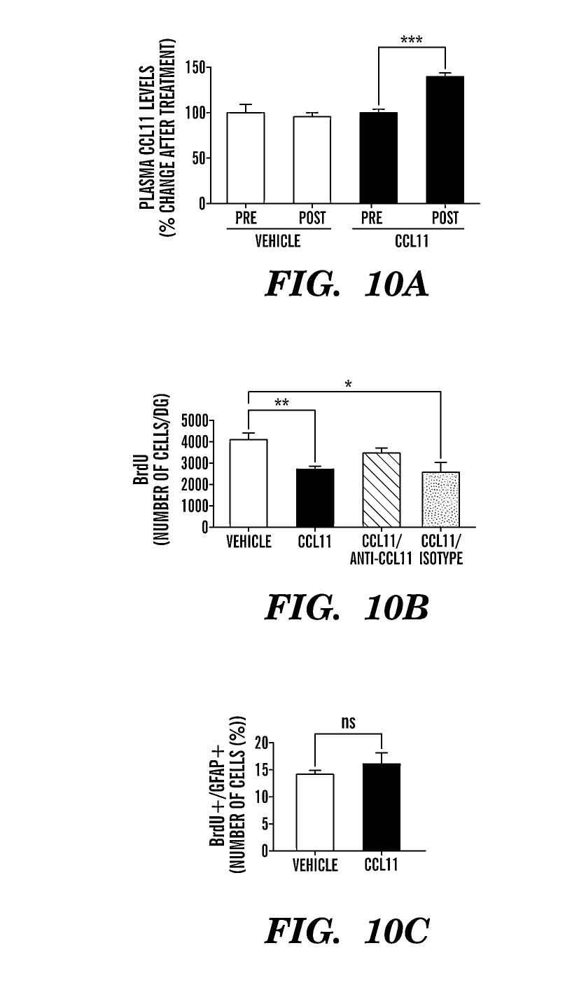

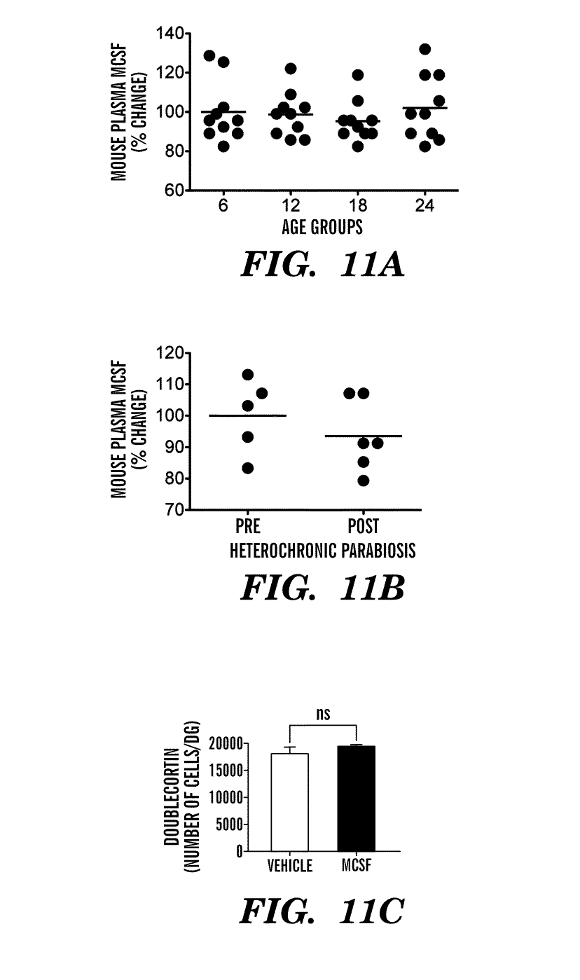

To confirm and expand upon this in vivo bioluminescent model, we next investigated the effect of systemic CCL11 on adult hippocampal neurogenesis using immunohistochemical analysis. In an independent cohort of young wild type adult mice, we administered recombinant CCL11 or vehicle alone, and in combination with either an anti-CCL11 neutralizing antibody or an isotype control antibody through intraperitoneal injections. The systemic administration of recombinant CCL11 induced an increase in CCL11 plasma levels (FIG. 10A), and caused a significant decrease in the number of Dcx-positive cells in the DG compared to mice injected with vehicle control, consistent with in vivo bioluminescent results (FIG. 4C). Importantly, this decrease in neurogenesis could be rescued by systemic neutralization of CCL11 (FIG. 4C). Likewise, BrdU-positive cells also showed similar changes in cell number (FIG. 10C), and furthermore the percentage of cells expressing both BrdU and NeuN decreased after systemic administration of CCL11 (FIG. 4D). The percentage of cells expressing BrdU and GFAP did not significantly change (FIG. 10C). As a negative control we assayed neurogenesis in a cohort of young adult mice after systemic administration of monocyte colony stimulating factor (MCSF), a protein measured in both of our independent proteomic screens that did not show an age-dependent change in plasma levels or a correlation with reduced neurogenesis, and detected no change in Dcx-positive cells in the DG (FIG. 11A-11C). Together, these data indicate that increasing the systemic level of CCL11, an individual age-related factor identified in our unbiased screen, is sufficient to partially recapitulate some of the inhibitory effects on neurogenesis observed with aging and heterochronic parabiosis.

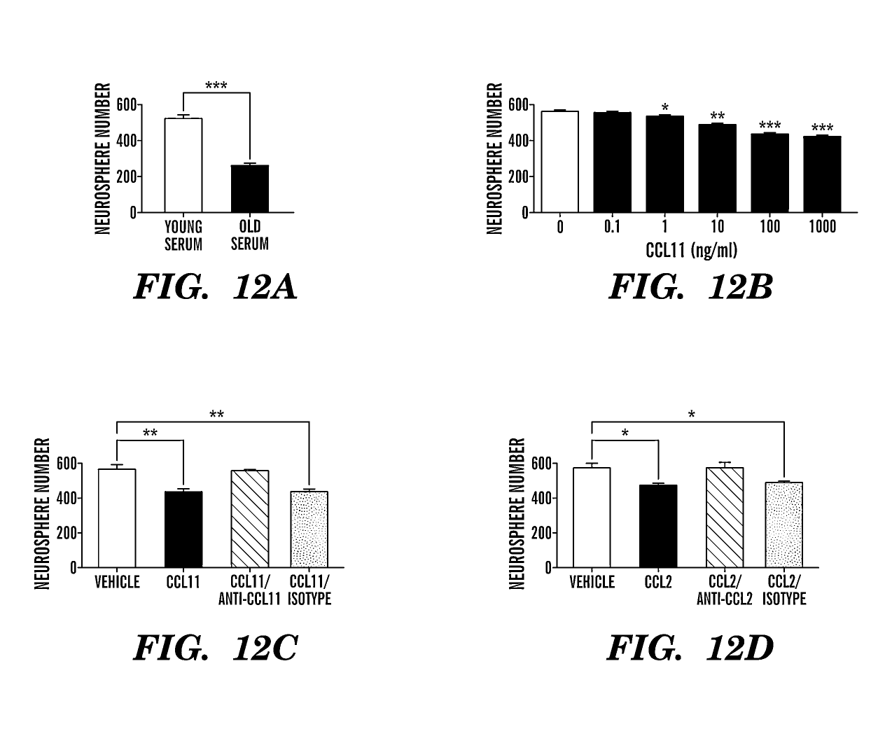

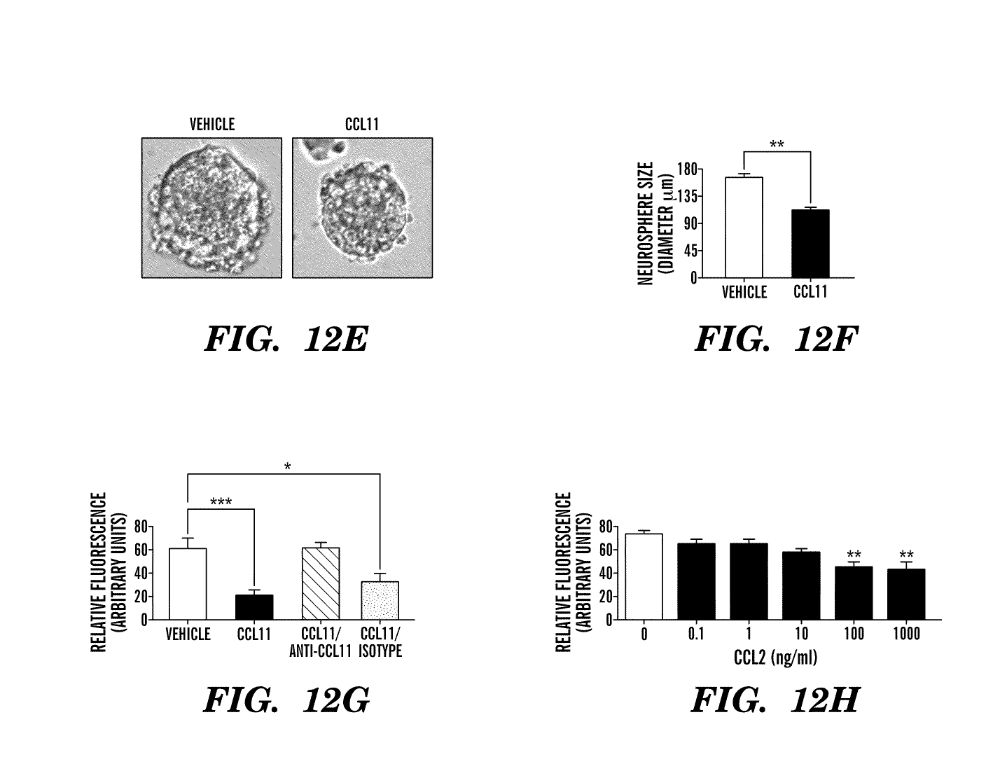

To investigate the possibility that age-related blood borne factors can directly influence stem cell function, we used primary mouse NPC cultures as a model of neural stem cell activity. We observed a 50% decrease in the number of neurospheres formed after a four-day exposure of NPCs to aged mouse serum when compared to NPCs exposed to young serum (FIG. 12A). We then tested whether the identified chemokines could also exert an inhibitory effect on NPCs and neural differentiation in vitro. The number of neurospheres formed from primary NPCs significantly decreased in the presence of either recombinant CCL11 (FIG. 12B-12C) or CCL2 (FIG. 12D). Additionally, neurosphere size also decreased in the presence of CCL11 (FIG. 12E-12F). Using a human derived NTERA cell line expressing eGFP under the Doublecortin promoter, we assayed neural differentiation and observed a significant decrease in eGFP expression after twelve days in culture with either CCL11 (FIG. 12G) or CCL2 (FIG. 12H) under differentiation conditions. Our data demonstrate that inhibitory factors present in aged blood are sufficient to act directly on NPCs in vitro. While these findings, together with studies showing a lack of a classical BBB in the neurogenic niche 13-15, open the possibility of a direct interaction of systemic factors with progenitor cells in vivo during aging, they do not preclude the possibility that age-related systemic factors may also act indirectly by stimulating other cell types that comprise the neurogenic niche to release additional inhibitory factors.

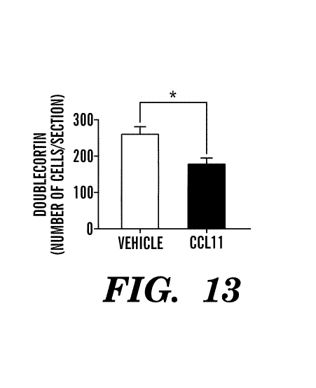

To examine the direct effect of CCL11 on neurogenesis in the brain, we stereotaxically injected recombinant CCL11 into the DG of young adult mice, and observed a decrease in the number of Dcx-positive cells when compared with the contralateral DG receiving vehicle control (FIG. 13). Furthermore, as an additional test of direct actions of systemic factors in the brain, we examined whether the inhibitory effect of peripheral CCL11 on neurogenesis could be restored locally by inhibiting CCL11 action specifically within the hippocampus. To test this, we stereotaxically injected CCL11-specific neutralizing antibody into the DG and isotype control antibodies into the contralateral DG of young adult mice. Following stereotaxic injection, we systemically administered either recombinant CCL11 or vehicle control by intraperitoneal injections. The decrease in Dcx-positive cell number observed in animals receiving systemic CCL11 administration could be rescued by neutralizing CCL11 within the DG with antigen specific antibodies but not isotype controls (FIG. 4E-4F), suggesting that increases in systemic chemokine levels exert a direct effect in the CNS.

Finally, to determine the physiological relevance of increased systemic CCL11 levels in mice we assessed hippocampal dependent learning and memory using the RAWM paradigm (FIG. 2D). Cohorts of young adult mice received intraperitoneal injections of recombinant murine CCL11 or PBS vehicle as a control. All mice showed similar spatial learning capacity during the training phase regardless of treatment (FIG. 4G). However, by the end of the testing phase animals receiving recombinant CCL11 protein exhibited impaired learning and memory deficits, committing significantly more errors in locating the target platform than animals receiving vehicle control (FIG. 4G). Together, these functional data demonstrate that increasing the systemic level of CCL11 not only inhibit adult neurogenesis but also impair hippocampal dependent learning and memory.

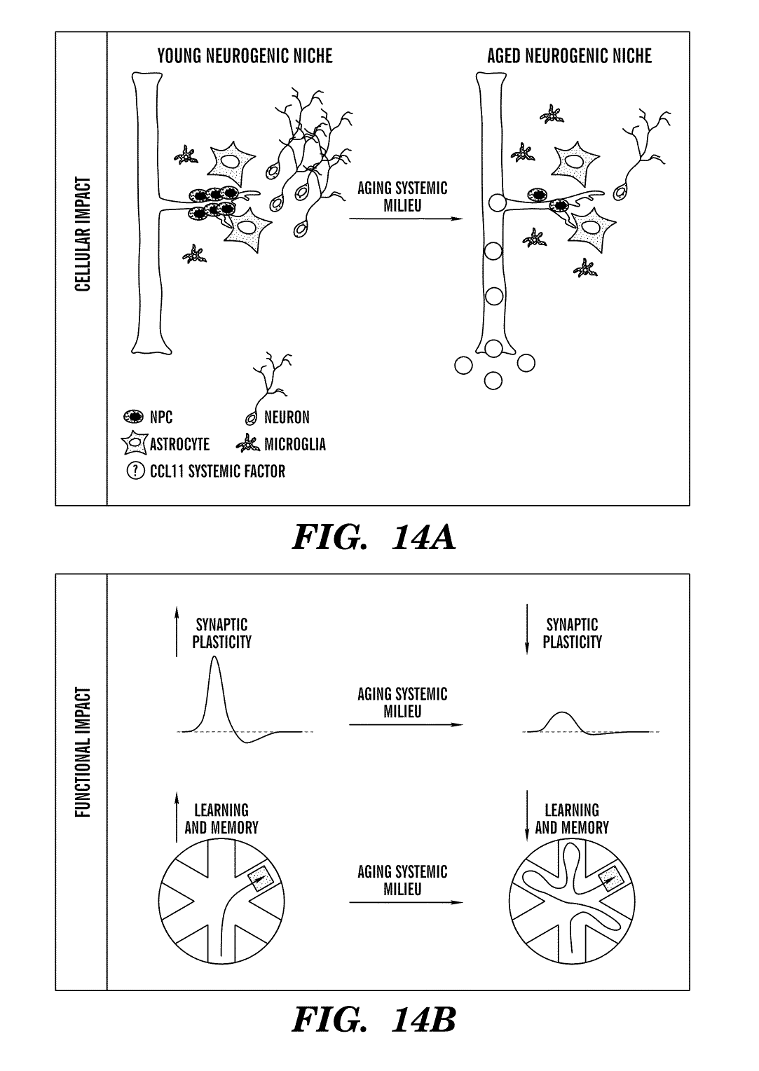

Cumulatively, our data link age-related molecular changes in the systemic milieu to the age-related decline in adult neurogenesis and associated impairments in synaptic plasticity and cognitive function observed during aging (FIGS. 14A-14B). We demonstrate that the influence of the aging systemic milieu is significant, and one that changes in an age-dependent fashion, potentially contributing to the susceptibility of the aging brain to cognitive impairments. The proteomic platform we used here was suitable to identify age-related systemic factors which inhibit adult neurogenesis.

In the adult brain, immune signaling is quickly emerging as one of the influential variables modulating stem cell function (Carpentier, P. A. & Palmer, T. D., Neuron 64 (1), 79-92 (2009); Tavazoie, M. et al., A specialized vascular niche for adult neural stem cells. Cell Stem Cell 3 (3), 279-288 (2008)) and neurodegeneration (Carpentier, P. A. & Palmer, T. D., Neuron 64 (1), 79-92 (2009); Lucin, K. M. & Wyss-Coray, T., Neuron 64 (1), 110-122 (2009); Monje, M. L., Toda, H., & Palmer, T. D., Science 302 (5651), 1760-1765 (2003)). However, to date most research has focused on the effect of brain-derived signaling proteins on adult neurogenesis (Carpentier, P. A. & Palmer, T. D., Neuron 64 (1), 79-92 (2009)), while the influence of the systemic milieu has been poorly investigated. We now show that an increase in the systemic levels of immune-related factors present in old blood is capable of diminishing adult neurogenesis and impairing spatial learning and memory. We identified age-related chemokines classically involved in peripheral inflammatory responses as biologically relevant inhibitory factors of neurogenesis in cell culture and in the CNS. Interestingly, CCL2, CCL11 and CCL12 are localized to within 70 kB on mouse chromosome 11, and likewise, CCL2 and CCL11 are within 40 kB on human chromosome 17 (mouse CCL12 is a homologue of human CCL2 and does not exist in humans), implicating this genetic locus in normal brain aging and possibly aging in general. Indeed, work investigating cellular senescence, a known hallmark of aging, furthers the involvement of some of the individual systemic chemokines reported here (CCL2) in the aging process as components of the Senescence-Associated Secretory Phenotype (Fumagalli, M. & d'Adda di Fagagna, F., Nat Cell Biol 11 (8), 921-923 (2009)).

While previous studies investigating the effect of the aging systemic milieu on peripheral tissue specific stem cell niches have alluded to the existence of ‘aging’ and ‘rejuvenating’ factors present in blood (Conboy, I. M. et al., Nature 433 (7027), 760-764 (2005)), such factors had not yet been identified. By describing the effect of an age-related systemic chemokine on the brain, our study introduces an important approach for the future discovery of other ‘aging’ and ‘rejuvenating’ factors. In focusing our unbiased proteomic screen on secreted signaling proteins in plasma that comprise a key part of the systemic milieu (that we collectively termed the communicome (Ray, S. et al., Nat Med 13 (11), 1369-1362 (2007)) we provide a more targeted platform for investigating age-related molecular changes and their functional role in aging tissues.

### The Neurogenic Niche

In the CNS, aging results in a decline in adult NPCs and neurogenesis. Stem cells and neurogenesis in the adult CNS have been observed in mammals including rodent, primates and humans primarily in the subventricular zone (SVZ) of the lateral ventricles and the subgranular zone (SGZ) of the hippocampus[5-10]. Adult neural stem cells are a relatively quiescent population that can both self-renew and give rise to more rapidly dividing progenitors that in turn produce neurons (neurogenesis), as well as astrocytes and oligodendrocytes (gliogenesis)[11, 12]. Ultimately, newly born neurons in the SVZ migrate and incorporate in the olfactory bulb where they are thought to mediate olfaction[13, 14]. In a similar manner, neurons born in the SGZ become granule neurons that integrate into the existing circuitry of the hippocampus and may directly influence learning and memory[15-18].

Adult NPCs are not distributed throughout the CNS randomly, they are rather centralized to local microenvironments, or neurogenic niches[18-20]. These niches are composed of surrounding cells such as astrocytes and oligodendrocytes, soluble factors, membrane bound molecules and extracellular matrix molecules that together are hypothesized to provide the permissive cues necessary for NPC maintenance, differentiation, and neural integration into the circuitry of the brain[21-23]. The neurogenic niche is exclusively concentrated around blood vessels, which allows for the communication with the systemic environment[18, 21, 22]. Moreover, blood vessels in the SVZ are closely associated with the basal lamina and are thought to modulate cytokines and growth factor availability in the neurogenic niche[12, 21]. Emerging evidence using three-dimensional imaging techniques has also suggested that neurovascular interactions in the neurogenic niche may have a functional significance. These studies indicate that contacts between NPCs and blood vessels are permeable, denude of astrocytic endfeet and a pericyte sheath. These features may indicate an intact blood-brain-barrier [24-27]. The absence of a classical BBB potentially enables circulating molecules from the periphery to access the neurogenic niche. To date while adult NPC populations and their respective microenvironments have been characterized, little is known about both the intrinsic and extrinsic regulation of NPCs during the aging process.

### Neural Stem Cells and Aging

Aging in mammals is associated with a global decline in the function and regenerative capacity of tissue specific stem cells[3, 28-31]. In the brain the cellular and molecular composition of the neurogenic niche is dynamically altered during aging (FIG. 1). The number of adult NPCs, and subsequently neurogenesis, has been observed to dramatically decline with age[30, 32, 33], while neuroinflammation increases. Additionally, the decline in NPC function may also be linked to sensory and cognitive impairments[4, 34-37].

### Intracellular Mechanisms

Adult NPC regulation may be divided at three distinct levels: (1) an intrinsic cellular clock that limits the potential number of cellular divisions, (2) intra- or extracellular factors that induce cell cycle arrest to maintain a pool of viable quiescent stem cells, and (3) the molecular composition of the neurogenic niche to either enhance or mitigate cellular proliferation[38, 39]. The investigation has begun on the regulation of NPC function by intrinsic cellular molecular mechanisms during aging. For example, recent studies in the aged brain have demonstrated that the decline in SVZ progenitor function and olfactory bulb neurogenesis may be partially mediated by increasing expression of p16^{INK4a}, a cycline-dependent kinase inhibitor linked to senescence mechanisms[40]. Moreover, premature senescence of NPCs can be promoted via the disregulation of the polycomb gene Bmi-1 signaling pathway[41]. Additionally, a forkhead transcription factor known to promote lifespan, FoxO3a, was implicated in the maintenance of NPC populations in both the SVZ and SGZ of the aging brain[42]. While such work begins to address how age-dependent changes to intrinsic CNS cues influence the regulation of NPC function, little was known as to how changes of the systemic environment (e.g., cues extrinsic to the CNS delivered by blood) to the molecular composition of the neurogenic niche can alter and impair NPC function during aging.

### The Systemic Milieu

Aging studies in muscle have shown the possibility of peripheral systemic factors in the regulation of stem cell function with age. For example, in vivo exposure of old muscle progenitors to factors from a young peripheral environment, mediated through systemic chimerism of young and old vasculatures, resulted in the rejuvenation of aged progenitors[3]. Accordingly, changes made to the aging peripheral milieu of adult animals via exercise or dietary restriction may also result in increased levels of neural progenitor proliferation and neurogenesis[43-49]. For example, the enhancement observed with increased exercise may be mediated in part by elevated levels of circulating growth factors such as vascular endothelial growth factor (VEGF) and insulin-like growth factor 1 (IGF-1), which may directly mediate NPC proliferation[50-53]. As another example, levels of IGF-1 decrease with age and the restoration to levels resembling a younger environment up-regulate neurogenesis and improve learning[54, 55]. In an additional example, MRI measurements of cerebral blood volume (CBV) in the hippocampus demonstrated that exercise selectively increases the CBV of the dentate gyrus[56]. Additionally, exercise-induced increases in the dentate gyrus CBV were found to correlate with postmortem increase in neurogenesis [56]. While rejuvenating effects of heterochronic parabiosis observed in muscle progenitors of old mice were attributed in part to intracellular mechanisms involving Notch signaling¹⁷, individual systemic factors associated with aging and tissue degeneration have not yet been characterized or investigated for their role in regulating the decline in tissue regeneration. In the present invention, changes in extrinsic cues from the systemic environment, particular individual systemic factors, are demonstrated to indicate and/or regulate adult NPC function, such as stem cell activity and tissue degeneration capacities.

### DETAILED EMBODIMENTS

Embodiments of the invention are based on the discovery of biomarkers that are capable of characterizing age-related changes in organisms, in particular CNS, such as reduced neurogenesis. For example, changes in the molecular composition of plasma are used as a means to model and predict the general aging process, as well as characterize more specific age-related changes in the nervous system such as reduced neurogenesis. In one embodiment, one or more biomarkers identified by the proteomic analysis described herein are systemic biomarkers indicating the age-dependent decline in neurogenesis. Thus, the embodiments of the present invention provide insight into how molecular changes in the systemic milieu influence the decline in NPC function observed during aging. In another embodiment, parabiotic pairings, i.e., a circulatory system is shared between young and old mice, are used to demonstrate that systemic factors naturally changing during aging can decrease neurogenesis in the young brain while increasing it in the aged brain. Targeted proteomic screens were employed to identify plasma signaling proteins that correlate with reduced neurogenesis observed in normal aging and/or heterochronic parabiosis. For example, β2M was recognized as a plasma signaling protein that can directly inhibit NPC function and neurogenesis both in vitro and in vivo.

### DEFINITION OF TERMS

As used herein, the term “treat” or “treatment” refers to reducing, alleviating, ameliorating, and/or stabilizing at least one adverse effect or symptoms, as well as delay in progression of symptoms of an age-associated disorder or disease. For example, “treatment” of a particular age-associated central nervous system disorder includes any one or more of: elimination of one or more symptoms of the age-associated central nervous system disorders, reduction of one or more symptoms of the age-associated central nervous system disorder, stabilization of the symptoms of the age-associated central nervous system disorder (e.g., failure to progress to more advanced stages of the age-associated central nervous system disorder), and delay in progression of one or more symptoms of the age-associated central nervous system disorder.

The term “subject” as used herein includes, without limitation, mammals, such as humans or non-human subjects. Non-human subjects may include primates, farm animals, sports animals, rodents or pets. In one embodiment, the subject is a human. In another embodiment, the subject is a non-human. Exemplary non-human subjects include, but not limited to, a monkey, ape, horse, cattle, pig, mouse, rat, dog, cat, or guinea pig.

As used herein, “biological fluid sample” encompasses a variety of fluid sample types obtained from a subject. The definition encompasses any fluid samples of a biological origin, including, but not limited to, blood, cerebral spinal fluid (CSF), urine, sputum, tears, lymph, and other liquid samples. The definition also includes samples that have been manipulated in any way after their procurement, such as by treatment with reagents, solubilization, or enrichment for certain components, such as proteins or polynucleotides. As used herein, the term “peripheral biological fluid sample” refers to a biological fluid sample that is not derived from the central nervous system. A “blood sample” is a biological sample which is derived from blood, such as peripheral (or circulating) blood. A blood sample may be, for example, whole blood, plasma or serum.

As used herein, a “reference value” or “reference level” can be an absolute value; a relative value; a value that has an upper and/or lower limit; a range of values; an average value; a median value, a mean value, or a value as compared to a particular control or baseline value. A reference value can be based on an individual sample value, such as for example, a value obtained from a sample from the subject being tested, but at an earlier point in time. The reference value can be based on a large number of samples, such as from population of subjects of the chronological age matched group, or based on a pool of samples including or excluding the sample to be tested.

### Proteomic Screening of Biomarkers

In one aspect, provided herein are proteomic approaches to identify one or more biomarkers useful for modeling and predicting the general aging process, as well as aiding in diagnosis, monitoring, predicting, and/or treating an age-associated disorder or disease. In one embodiment, levels of a group of biomarkers are obtained for a set of biological fluid samples, in particular peripheral biological fluid samples from one or more healthy subjects. The samples are selected such that they can be segregated into one or more groups on the basis of chronological ages. For example samples may be grouped into different age groups with an interval of ages between succeeding age groups being any where from 1 year to 50 years. For example, samples may be grouped into chronological age groups of 21-30, 31-40, 41-50, 51-60, 61-70, 71-80, 81-90, 91-100 years. Alternatively, samples may be grouped into a younger age group and an older age group. In one embodiment, samples are grouped into an older group (65-90 years) and a younger group (20-45 years). The ages between different age groups may or may not be in succession. The measured values from the samples from one age group are compared to samples from other age group(s) to identify those biomarkers which differ significantly amongst the different age groups. Those biomarkers that vary significantly amongst the different age groups may then be used in methods for modeling and predicting the general aging process, as well as aiding in diagnosis, monitoring, predicting, and/or treating an age-associated disorder or disease.

In one embodiment, measured values for a set of peripheral biological fluid samples from one or more healthy subjects from one or more chronological groups are compared, wherein biomarkers that vary significantly are used. In another embodiment, levels of a set of peripheral biological fluid samples from one or more healthy subjects from one or more chronological groups are measured to produce measured values, wherein biomarkers that vary significantly are used.

Accordingly, in one embodiment, provided are methods for identifying one or more biomarkers which can be used to diagnose an age-associated disorder, the method comprising providing measured values for a plurality of biomarkers from a set of biological fluid samples of a population of healthy subjects without said age-associated disorder, wherein the set of biological fluid samples is divisible into groups on the basis of chronological ages of the subjects, comparing the measured values from each chronological age group for at least one biomarker, and identifying at least one biomarker for which the measured values are significantly different between the different chronological age groups.

In one embodiment, provided are methods for identifying one or more biomarkers which can be used to detect diminished cell activity, the method comprising providing measured values for a plurality of biomarkers from a set of biological fluid samples of a population of healthy subjects having normal cell activity, wherein the set of biological fluid samples is divisible into groups on the basis of chronological ages of the subjects, comparing the measured values from each chronological age group for at least one biomarker, and identifying at least one biomarker for which the measured values are significantly different between the different chronological age groups.

In one embodiment, provided are methods for identifying one or more biomarkers which can be used to detect diminished tissue regeneration capacity, the method comprising providing measured values for a plurality of biomarkers from a set of biological fluid samples of a population of healthy subjects having normal tissue regeneration capacity, wherein the set of biological fluid samples is divisible into groups on the basis of chronological ages of the subjects, comparing the measured values from each chronological age group for at least one biomarker, and identifying at least one biomarker for which the measured values are significantly different between the different chronological age groups.

In one embodiment, provided are methods for identifying one or more biomarkers which can be used to identify a medical treatment or medication for promoting cell activity, increasing tissue regeneration capacity or treating an age-associated disorder, the method comprising providing measured values for a plurality of biomarkers from a set of biological fluid samples of a population of healthy subjects, wherein the set of biological fluid samples is divisible into groups on the basis of chronological ages of the subjects, comparing the measured values from each chronological age group for at least one biomarker, and identifying at least one biomarker for which the measured values are significantly different between the different chronological age groups.

In one embodiment, provided are methods for identifying one or more biomarkers which can be used to monitor the effect of a medical treatment or medication for promoting cell activity, increasing tissue regeneration capacity or treating an age-associated disorder, the method comprising providing measured values for a plurality of biomarkers from a set of biological fluid samples of a population of healthy subjects, wherein the set of biological fluid samples is divisible into groups on the basis of chronological ages of the subjects, comparing the measured values from each chronological age group for at least one biomarker, and identifying at least one biomarker for which the measured values are significantly different between the different chronological age groups.

The process of comparing the measured values may be carried out by any method known in the art, including Significance Analysis of Microarrays, Tree Harvesting, CART, MARS, Self Organizing Maps, Frequent Item Set, or Bayesian networks. In some embodiments, the comparing process is carried out using Significance Analysis of Microarrays.

The biological fluid samples, including peripheral biological fluid samples, and/or CSF sample, that derived from one or more healthy subjects. The subject may be a mammal, such as humans or non-human subjects. In some embodiments, the biological fluid sample is peripheral biological fluid sample, such as blood samples, for example, a plasma sample. Biomarkers measured in the embodiments of the present invention may be any proteinaceous biological marker found in a biological fluid sample. Table 1 and 2 contain a collection of exemplary biomarkers from human plasma and mouse plasma, respectively, and Table 3 contains a collection of exemplary biomarkers from human CSF. Additional biomarkers are described herein in the Examples.

The age-associate disorders or diseases can be disorders or diseases associated with any organism. In some embodiments, the age-associated disease is any neurodegenerative disease such as Alzheimer's disease, Huntington's disease or Parkinson's disease. In some embodiments, the age-associated disease is a neuroinflammatory disease.

The cell activities to be detected or monitored include cell proliferation, self-renewal, or differentiation. In some embodiments, the biomarkers are identified to detect diminished stem cell or progenitor cell activities. In some embodiment, the cell is a neuronal cell or glial cell. In some embodiments, the stem cell or progenitor cell is a neural stem cell or neural progenitor cell.

In some embodiments, the biomarkers are identified to detect diminished neural tissue renegeration capacity.

### Age-Associated Disorder or Disease and Reduced Neural Cell Regeneration or Impaired Cognitive Function-Associate Disorder or Disease

As used herein, the term “age-associated disorder” or “age-associated disease” and refers to a disorder or disease that is seen with increased frequency upon aging. Age-associated disorder or disease general includes, without limitation, CNS disorders or diseases, cardiovascular system disorders or diseases, autonomic nervous system disorders or diseases, eye and ear disorders or diseases, respiratory system disorders or diseases, gastrointestinal system disorders or diseases, renal disorders or diseases, genitourinary system disorders or diseases, endocrine system disorders or diseases, hematological and immune system disorders or diseases, muscular skeletal system disorders or diseases, cancer or drug metabolism disorders.

Reduced neural cell regeneration or impaired cognitive function-associate diseases or disorders are defined identically with the “age-associated disorder” or “age-associated disease” in the context of this application.

In some embodiments, the age-associated disorder or disease is a CNS disorder. Age-associated CNS disorder may be neurologic disorder or psychiatric disorder. Age-associated neurologic disorder may have the symptoms such as impairment of memory, decreased cognitive or intellectual functions, deterioration of mobility (e.g., change in gait), altered sleep pattern, decreased sensory input (visual, acoustic, taste, smell, etc.), or autonomic nerve system imbalance. Age-associated psychiatric disorder may have the symptoms such as depression, dementia, confusion, catatonia or delirium.

In some embodiments, the age-associated CNS disorder or disease includes depression, dementia, depression, delirium, memory impairment, cognitive or intellectual functions impairment, deterioration of mobility, altered sleep pattern, decreased sensory input, autonomic nerve system imbalance, Amyotrophic lateral sclerosis, mild cognitive impairment, Alzheimer's disease, Huntington's disease or Parkinson's disease.

In some embodiments, the age-associated CNS disorder or disease is neurodegenerative disorder or disease. In some embodiments, the CNS disorder does not contain cognitive or intellectual functions impairment. In one embodiment, the age-associated central nervous system disorder does not contain mild cognitive impairment or Alzheimer's disease.

### Age-Associated Biomarkers

“Age-associated biomarker” refers to a biomarker that is an indicator of an age-associated disorder or disease; and it may also refer to a biomarker that is in age-specific pattern and is an indicator of the biological age of a subject. “Biological age” of a subject or individual as used herein is the same as those commonly understood to one skilled in the art. Biological age of a subject is a relative term and may be or may not be the same as the chronological age of the subject. Determining biological age or healthy age of a subject can be to determine how much the subject body as a whole has aged with time or how young or how old a subject is compared to its chronological-matched peers. “Age-associated biomarker”, “age-related biomarker”, and “biomarker” (used interchangeably herein) are terms of convenience to refer to the markers described herein and their use. As this disclosure makes clear, these biomarkers are useful for, for example, diagnosing an age-associated disorder; assessing risk of developing an age-associated disorder; detecting diminished cell activity such as NPC activity; detecting diminished tissue regeneration capacity; identifying a medical treatment or medication for promoting NPC activity, for increasing tissue regeneration capacity or for treating an age-associated disorder; monitoring the effect of such medical treatment or medication on a subject; or identifying a candidate agent for modulating the activity or expression of the biomarker, which may be useful as drug agents to prevent or treat an age-associated disorder. An age-associated disorder or disease is defined herein, such as a neurodegenerative disease or neuroinflammatory disease.

Age-associated biomarkers include but are not limited to secreted proteins or metabolites present in a subject's biological fluids (that is, a biological fluid sample), such as for example, blood, including whole blood, plasma or serum; urine; cerebrospinal fluid; tears; saliva; and sputum. Biological fluid samples encompass clinical samples, and also includes serum, plasma, and other biological fluids. A blood sample may include, for example, various cell types present in the blood including platelets, lymphocytes, polymorphonuclear cells, macrophages, erythrocytes.

In one aspect, the expression pattern of the biomarkers provided here changes during the aging process. Hence the biomarkers for healthy aging are useful to model and predict the relative biological age. “Biological age” of a subject or individual as used herein is the same as those commonly understood to one skilled in the art. Biological age of a subject is a relative term and may be or may not be the same as the chronological age of the subject. Determining biological age or healthy age of a subject is to determine how much the subject body as a whole has aged with time or how young or how old a subject is compared to its chronological-matched peers. This is useful, for example, to predict increase and/or decrease in the risk of developing an age-associated disorder or disease and to monitor the age-associated disorder or disease. These biomarkers include, but are not limited to α-2 Macroglobulin, Apoliporotein H (ApoH), β-2 Microglobulin (β2-M), Basic fetal growth factor (bFGF), Complement factor 3 (C3), Cancer antigen 125 (CA125), Calcitonin, Carcinoembrionic antigen (CEA), CCL11/Eotaxin, CCL2/MCP-1, CCL22/MDC, CCL4/MIP-1β, CD40, CXCL5/ENA-78, Endothelin-1, Erythropoietin (Epo), Extracellular newly identified RAGE-binding protein (EN-RAGE), Fatty acid binding protein 3 (FABP3), Fibrinogen α/β/γ chain, Growth hormone (GH1), Haptoglobin (HP), Intercellular adhesion molecule 1 (ICAM-1), IgE, IgM, Interleukin 1α (IL-1α), Interleukin 1β (IL-1β), Interleukin 6 (IL-6), Interleukin 16 (IL-16), Interleukin 18 (IL-18), Insulin-like growth factor 1 (IGF-I), Lipoprotein A (LPA), Monocyte colony stimulating factor (M-CSF), Matrix metalloproteinase 2 (MMP-2), Matrix metalloproteinase 9 (MMP-9), Myeloperoxidese (MPO), Myoglobin, Plasminogen activator inhibitor 1 (PAI-1), Sex hormone-binding globulin (SHBG), Tissue inhibitor of metalloproteinase 1 (TIMP-1), Tumor necrosis factor-α (TNF-α), Tumor necrosis factor receptor II (TNFR-2), Vascular cell adhesion molecule 1 (VCAM-1), and Vascular endothelial growth factor (VEGF), XCL1/Lymphotactin. These biomarkers are secreted proteins in peripheral biological fluids of a subject, such as human. One or more of these biomarkers can be used to predict age of a human. For example, the 44 predictors detected in human plasma in Table 1 significantly changed expression levels in the older group (75-88 years) in comparison to the younger group (20-44 years), as shown in Example 1. In one embodiment, these age-associated biomarkers include, but are not limited to Adiponectin/Acrp30, Apolipoprotein A-1 (ApoA1), β-2 Microglobulin ((2-M), CCL11/Eotaxin, CD40, Ferritin H+L chain, Fibrinogen α/β/γ chain, Prostate specific antigen, free (PSA), Tissue inhibitor of metalloproteinase 1 (TIMP-1), and Vascular cell adhesion molecule 1 (VCAM-1). For example, these 10 age-associate biomarkers detected in human plasma in Table 1 may be used to predict biological ages of healthy donors that match their chronological ages in several different scenarios, as shown in Example 1.

In one embodiment, the age-associated biomarkers include, but are not limited to Apolipoprotein A-1 (ApoA1), β2M, Calbindin, CCL2/MCP-1, CCL3/MIP-1α, CCL5/RANTES, CCL7/MCP-3, CCL9/10/MIP-1γ, CCL11/Eotaxin, CCL12/MCP-5, CCL19/MIP-3β, CCL22/MDC, CD40, CD40L, Clusterin, C reactive protein (CRP), CXCL1, 2, 3/GRO-α,β,γ, CXCL6/GCP-2, CXCL10/IP-10, Cystatin-C, Endothelial growth factor (EGF), Endothelin-1, Factor VII (FVII), Growth hormone (GH1), Glutathion S-transferase (GSTa1), Haptoglobin (HP), IgA, Interleukin 1α (IL-1α), Interleukin 1β (IL-1β) Interleukin 5 (IL-5), Interleukin 6 (IL-6), Interleukin 10 (IL-10), Interleukin 18 (IL-18), Insulin, Leptin, Leukemia inhibitory factor (LIF), Lipocalin-2, Monocyte colony stimulating factor (M-CSF), Matrix metalloproteinase 9 (MMP-9), Myoglobin, Osteopontin, Serum Amyloid P (SAP), Serum glutamic oxaloacetic transaminase (SGOT), Tissue factor (TF), Tissue inhibitor of metalloproteinase 1 (TIMP-1), Thrombopoietin (Tpo), Vascular cell adhesion molecule 1 (VCAM-1), Vascular endothelial growth factor (VEGF), Von Willebrand factor (vWF), XCL1/Lymphotactin. These biomarkers are secreted proteins in peripheral biological fluids of a subject, such as mouse. For example, Table 2 provides a listing of age-associated biomarkers that are sufficiently detectable in mice plasma samples. In one embodiment, the age-associated biomarkers include one or more of Apolipoprotein A-1 (ApoA1), β-2 Microglobulin ((2-M), CCL2/MCP-1, CCL11/Eotaxin, CD40, Growth hormone (GH1), Haptoglobin (HP), Interleukin 18 (IL-18), Monocyte colony stimulating factor (M-CSF), Myoglobin, Tissue inhibitor of metalloproteinase 1 (TIMP-1), and Vascular cell adhesion molecule 1 (VCAM-1). These biomarkers are secreted proteins in peripheral biological fluids of a subject. In some examples, these biomarkers are shared across species to monitor age in several different scenarios. For example, these 12 protein markers detected from healthy mice plasma samples re capable of modeling ages with clear separations, as shown in Example 2, and Table 2. In one embodiment, the age-associate biomarkers include one or more of CCL2/MCP-1, CCL11/Eotaxin, CXCL10/IP-10, Interleukin 10 (IL-10), Serum glutamic oxaloacetic transaminase (SGOT), and Von Willebrand factor (vWF). In one embodiment, the age-associate biomarkers include one or more of 132-M, CCL2/MCP-1, and CCL11/Eotaxin.

In one embodiment, the age-associated biomarker include, but are not limited to α-1 Antitrypsin, α-2 Macroglobulin, α-Fetoprotein, Adiponectin/Acrp30, Apolipoprotein CIII (ApoC3), Apoliporotein H (ApoH), Apolipoprotein A-1 (ApoA1), β2M, Basic fetal growth factor (bFGF), Complement factor 3 (C3), Cancer antigen 19-9 (CA19-9), Calcitonin, CCL2/MCP-1, CCL3/MIP-1α, CCL4/MIP-1β, CCL5/RANTES, CCL11/Eotaxin, CD40, CD40L, Creatine kinase-MB (CK-MB), C reactive protein (CRP), CXCL5/ENA-78, CXCL8/IL-8, Endothelial growth factor (EGF), Endothelin-1, Erythropoietin (Epo), Extracellular newly identified RAGE-binding protein (EN-RAGE), Fatty acid binding protein 3 (FABP3), Ferritin H+L chain, Fibrinogen α/β/γ chain, Factor VII (FVII), Growth hormone (GH1), Glutathion S-transferase (GSTA1), Haptoglobin (HP), Intercellular adhesion molecule 1 (ICAM-1), IgA, IgM, Interleukin 1β (IL-1β), Interleukin 1 receptor antagonist (IL-1ra), Interleukin 4 (IL-4), Interleukin 5 (IL-5), Interleukin 6 (IL-6), Interleukin 7 (IL-7), Interleukin 10 (IL-10), Interleukin 12p70 (IL-12p70), Interleukin 13 (IL-13), Interleukin 15 (IL-15), Interleukin 16 (IL-16), Interleukin 18 (IL-18), Leptin, Matrix metalloproteinase 2 (MMP-2), Matrix metalloproteinase 3 (MMP-3), Myeloperoxidase (MPO), Myoglobin, Plasminogen activator inhibitor 1 (PAI-1), Prostatic acid phosphatase (PAP), Pregnancy associated plasma protein (PAPP-A), Prostate specific antigen, free (PSA), Stem cell factor (SCF), Serum Amyloid P (SAP), Serum glutamic oxaloacetic transaminase (SGOT), Sex hormone-binding globulin (SHBG), Thyroid stimulating hormone, α/β-subunit (TSH), Thyroxine binding globulin (TBG), Tissue factor (TF), Tissue inhibitor of metalloproteinase 1 (TIMP-1), Thrombopoietin (Tpo), Tumor necrosis factor-α (TNF-α), Tumor necrosis factor-β (TNF-β), Tumor necrosis factor receptor II (TNFR-2), Vascular cell adhesion molecule 1 (VCAM-1), Vascular endothelial growth factor (VEGF), and Von Willebrand factor (vWF). For example, Table 3 provides a listing of age-associated biomarkers that are detectable in cerebrospinal fluid samples, as shown in Example 1.

In one aspect, the biomarkers provided herein can be indicators of an age-associated disorder or disease and thus are age-associated disorder or disease markers. The age-associated disorder or disease markers include one or more of CCL2, Eotaxin/CCL11, β2M, MCP-1, MCP-5, and Haptoglobin. In one example, the age-associated disease biomarkers include one or more of Eotaxin/CCL11, β2M, and MCP-1.

In one embodiment, the age-associated disease biomarkers include at least β2M. β2M is a nonglycosylated protein with a secreted form composed of 100 amino acids[63]. It is synthesized by all nucleated cells and traditionally represents the light chain of the MHC class 1 molecules (MHC1), a part of the adaptive immune system that helps to discriminate cells as either of self origin or foreign. Independent of its classical role as part of the MHC1 complex in the adaptive immune system, soluble β2M has been shown to influence the biology of certain cells types in a pleomorphic manner [67, 68]. β2M has also exhibited some divergent age-dependent modes of regulation in the CNS [83-86]. However, the functional role of β2M in the aging brain has not yet been studied. Thus far the majority of studies on β2M reported in the CNS have focused on genetic mouse models in which β2M have been ablated throughout the body and at all stages of development. Systemic expression of β2M, and the subsequent analysis of differential roles for CNS-derived and systemically derived β2M on the declining regenerative capacity observed in adult NPCs in the aging brain have not been addressed. The embodiments of the present invention answer these questions. Accordingly, in some embodiments, β2M is used to assess risk of developing an age-associated disorder or disease, diagnose, and monitoring age-associated disorders or diseases, such as, neurodegenerative disease, neuroinflammatory disease, declined NPC functions or diminished tissue regeneration capacity.

In another embodiment, the age-associated disease biomarkers include at least Eotaxin/CCL11. Similarly as β2M, Eotaxin/CCL11 in the periphery is also classically involved in inflammatory immune responses. However, a functional role for Eotaxin/CCL11 in the CNS had not been identified. Systemic analysis of differential roles for CNS-derived and systemically derived Eotaxin/CCL11 on the declining regenerative capacity observed in adult NPCs in the aging brain have not been addressed. These issues have been addressed in the embodiments of the present invention. Accordingly, in some embodiments, Eotaxin/CCL11 is used to assess risk of developing an age-associated disorder or disease, diagnose, and monitoring age-associated disorders or diseases, such as, neurodegenerative disease, neuroinflammatory disease, declined NPC functions or diminished tissue regeneration capacity.

Although the levels of sensitivity and specificity with single biomarkers for practice of the age-associated disorder or disease diagnosis and treatment methods or practice of modeling aging process are acceptable, the effectiveness (e.g., sensitivity and/or specificity) of the methods of the age-associated disorder diagnosis methods of the present invention may be enhanced when more than one biomarker are utilized. In some examples, the methods of determining biological age or biological state of the present invention are generally enhanced when at least 2, 3, 4, or 5, 10, 12, 29, or 40, or 44 biomarkers are utilized. In some embodiments, the methods of the age-associated disorder or disease diagnosis and treatment methods of the instant invention are generally enhanced when at least 2, 3, 4, or more biomarkers are utilized. Typically, between 2-5, 2-10, or 2-12 markers, or 5-10 or 5-12 markers are analyzed to obtain enhanced diagnostic value. In some aspects, 1-4 markers are used and the set of markers comprise at least CCL2/MCP-1 and CCL11/Eotaxin.

Multiple biomarkers may be selected from the age-associated biomarkers disclosed herein by a variety of methods, including cluster analysis by selecting for cluster diversity. For example, age-associated biomarkers may be selected to preserve cluster diversity of selected proteins or sample diversity. The clusters are formed by qualitative measurements for each biomarker which are most closely correlated. For example, statistical method Elastic net may be used to analyze unit L2-norm standardized data from detectable protein markers to find markers that best characterize age. Elastic net method is a regularization and variable selection method that identifies significant correlations between variables of interest in a large number of observations (e.g., age or diagnosis correlated with results of proteomic microarrays or multiplex assays). An internal correction algorithm and, typically at least a 2, 3, 4, 5, 6, 7, 8, 9, or a 10-fold cross validation step assess and minimize classification error. This cluster analysis can produce a ranked list of markers to characterize age and a list of remaining variables that do not contribute to characterize age. Multiple biomarkers may be selected from the ranked list following the ranking as to enhance the effectiveness (e.g., sensitivity and/or specificity) of the methods of the present invention. An example of selecting 40 protein markers and 10 robust markers from human plasma donors to model age are presented in Example 1.

### Measuring Levels of Biomarkers

There are a number of statistical tests for identifying biomarkers which vary significantly between the different chronological age groups, such as Significance Analysis of Microarrays (SAM). The SAM technique assigns a score to each biomarker on the basis of change in expression relative to the standard deviation of repeated measurements. For biomarkers with scores greater than an adjustable threshold, the algorithm uses permutations of the repeated measurements to estimate the probability that a particular biomarker has been identified by chance (calculated as a “q-value”), or a false positive rate which is used to measure accuracy. The SAM technique can be carried out using publicly available software called Significance Analysis of Microarrays (see SAM 3.00 algorithm which is available from the world wide web at stat “dot” Stanford “dot” edu/˜tibs/SAM/index.htm).

A biomarker is considered “identified” when it is sufficiently or significantly different between the groups of biological samples tested. Levels of a biomarker are “sufficiently or significantly different” when the probability that the particular biomarker has been identified by chance is less than a predetermined value. The method of calculating such probability will depend on the exact method utilizes to compare the levels between the groups (e.g., if SAM is used, the q-value will give the probability of misidentification, and the p value will give the probability if the t test (or similar statistical analysis) is used). As will be understood by those in the art, the predetermined value will vary depending on the number of biomarkers measured per sample and the number of samples utilized. Accordingly, predetermined value may range from as high as about 50% to as low as about 20, 10, 5, 3, 2, or 1%.

In some aspects, when the expression of the biomarker is increased by about 50% or more compared to the reference value, it is considered increased.

As described herein, the level of at least one biomarker is measured in a biological sample from a subject. The biomarker level(s) may be measured using any available measurement technology that is capable of specifically determining the level of the biomarker in a biological sample. The measurement may be either quantitative or qualitative, so long as the measurement is capable of indicating whether the level of the biomarker in the biological fluid sample is above or below the reference value.

The measured level may be a primary measurement of the level a particular biomarker measuring the quantity of biomarker itself, such as by detecting the number of biomarker molecules in the sample) or it may be a secondary measurement of the biomarker (a measurement from which the quantity of the biomarker can be but not necessarily deduced, such as a measure of enzymatic activity (when the biomarker is an enzyme) or a measure of nucleic acid, such as mRNA, encoding the biomarker). Qualitative data may also be derived or obtained from primary measurements.

Biological fluid samples, particularly peripheral biological fluid samples may be tested without prior processing of the sample as allowed by some assay formats. Alternatively, many peripheral biological fluid samples will be processed prior to testing. Processing generally takes the form of elimination of cells (nucleated and non-nucleated), such as erythrocytes, leukocytes, and platelets in blood samples, and may also include the elimination of certain proteins, such as certain clotting cascade proteins from blood. In some examples, the peripheral biological fluid sample is collected in a container comprising EDTA. See Example 1 for additional sample collection procedures. Commonly, biomarker levels may be measured using an affinity-based measurement technology. “Affinity” as relates to an antibody is a term well understood in the art and means the extent, or strength, of binding of antibody to the binding partner, such as a biomarker as described herein (or epitope thereof). Affinity may be measured and/or expressed in a number of ways known in the art, including, but not limited to, equilibrium dissociation constant (K_{D} or K_{d}), apparent equilibrium dissociation constant (K_{D}′ or K_{d}′), and IC₅₀ (amount needed to effect 50% inhibition in a competition assay; used interchangeably herein with “I₅₀”). It is understood that, for purposes of this invention, an affinity is an average affinity for a given population of antibodies which bind to an epitope.

Affinity-based measurement technology utilizes a molecule that specifically binds to the biomarker being measured (an “affinity reagent,” such as an antibody or aptamer), although other technologies, such as spectroscopy-based technologies (e.g., matrix-assisted laser desorption ionization-time of flight, or MALDI-TOF, spectroscopy) or assays measuring bioactivity (e.g., assays measuring mitogenicity of growth factors) may be used. Affinity-based technologies may include antibody-based assays (immunoassays) and assays utilizing aptamers (nucleic acid molecules which specifically bind to other molecules), such as ELONA. Additionally, assays utilizing both antibodies and aptamers are also contemplated (e.g., a sandwich format assay utilizing an antibody for capture and an aptamer for detection).

Immunoassay technology may include any immunoassay technology which can quantitatively or qualitatively measure the level of a biomarker in a biological sample. Suitable immunoassay technology includes, but not limited to radioimmunoassay, immunofluorescent assay, enzyme immunoassay, chemiluminescent assay, ELISA, immuno-PCR, and western blot assay. Likewise, aptamer-based assays which can quantitatively or qualitatively measure the level of a biomarker in a biological sample may be used in the methods of the invention. Generally, aptamers may be substituted for antibodies in nearly all formats of immunoassay, although aptamers allow additional assay formats (such as amplification of bound aptamers using nucleic acid amplification technology such as PCR (U.S. Pat. No. 4,683,202) or isothermal amplification with composite primers (U.S. Pat. Nos. 6,251,639 and 6,692,918).

A wide variety of affinity-based assays are known in the art. Affinity-based assays will utilize at least one epitope derived from the biomarker of interest, and many affinity-based assay formats utilize more than one epitope (e.g., two or more epitopes are involved in “sandwich” format assays; at least one epitope is used to capture the marker, and at least one different epitope is used to detect the marker).

Affinity-based assays may be in competition or direct reaction formats, utilize sandwich-type formats, and may further be heterogeneous (e.g., utilize solid supports) or homogenous (e.g., take place in a sirigle phase) and/or utilize or immunoprecipitation. Many assays involve the use of labeled affinity reagent (e.g., antibody, polypeptide, or aptamer); the labels may be, for example, enzymatic, fluorescent, chemiluminescent, radioactive, or dye molecules. Assays which amplify the signals from the probe are also known; examples of which are assays which utilize biotin and avidin, and enzyme-labeled and mediated immunoassays, such as ELISA and ELONA assays. For example, the biomarker concentrations from biological fluid samples may be measured by LUMINEX® assay or ELISA, as described in Example 1. Either of the biomarker or reagent specific for the biomarker can be attached to a surface and levels can be measured directly or indirectly.

In a heterogeneous format, the assay utilizes two phases (typically aqueous liquid and solid). Typically a biomarker-specific affinity reagent is bound to a solid support to facilitate separation of the biomarker from the bulk of the biological sample. After reaction for a time sufficient to allow for formation of affinity reagent/biomarker complexes, the solid support or surface containing the antibody is typically washed prior to detection of bound polypeptides. The affinity reagent in the assay for measurement of biomarkers may be provided on a support (e.g., solid or semi-solid); alternatively, the polypeptides in the sample can be immobilized on a support or surface. Examples of supports that can be used are nitrocellulose (e.g., in membrane or microtiter well form), polyvinyl chloride (e.g., in sheets or microtiter wells), polystyrene latex (e.g., in beads or microtiter plates), polyvinylidine fluoride, diazotized paper, nylon membranes, activated beads, glass and Protein A beads. Both standard and competitive formats for these assays are known in the art. Accordingly, provided herein are complexes comprising at least one biomarker bound to a reagent specific for the biomarker, wherein said reagent is attached to a surface. Also provided herein are complexes comprising at least one biomarker bound to a reagent specific for the biomarker, wherein said biomarker is attached to a surface.

Array-type heterogeneous assays are suitable for measuring levels of biomarkers when the methods of the invention are practiced utilizing multiple biomarkers. Array-type assays used in the practice of the methods of the invention will commonly utilize a solid substrate with two or more capture reagents specific for different biomarkers bound to the substrate a predetermined pattern (e.g., a grid). The biological fluid sample is applied to the substrate and biomarkers in the sample are bound by the capture reagents. After removal of the sample (and appropriate washing), the bound biomarkers are detected using a mixture of appropriate detection reagents that specifically bind the various biomarkers. Binding of the detection reagent is commonly accomplished using a visual system, such as a fluorescent dye-based system. Because the capture reagents are arranged on the substrate in a predetermined pattern, array-type assays provide the advantage of detection of multiple biomarkers without the need for a multiplexed detection system.

In a homogeneous format the assay takes place in single phase (e.g., aqueous liquid phase). Typically, the biological sample is incubated with an affinity reagent specific for the biomarker in solution. For example, it may be under conditions that will precipitate any affinity reagent/antibody complexes which are formed. Both standard and competitive formats for these assays are known in the art.

In a standard (direct reaction) format, the level of biomarker/affinity reagent complex is directly monitored. This may be accomplished by, for example, determining the amount of a labeled detection reagent that forms is bound to biomarker/affinity reagent complexes. In a competitive format, the amount of biomarker in the sample is deduced by monitoring the competitive effect on the binding of a known amount of labeled biomarker (or other competing ligand) in the complex. Amounts of binding or complex formation can be determined either qualitatively or quantitatively.

The methods described in this patent may be implemented using any device capable of implementing the methods. Examples of devices that may be used include but are not limited to electronic computational devices, including computers of all types. When the methods described in the present invention are implemented in a computer, the computer program that may be used to configure the computer to carry out the steps of the methods may be contained in any computer readable medium capable of containing the computer program. Examples of computer readable medium that may be used include but are not limited to diskettes, CDROMs, DVDs, ROM, RAM, and other memory and computer storage devices. The computer program that may be used to configure the computer to carry out the steps of the methods may also be provided over an electronic network, for example, over the internet, world wide web, an intranet, or other network.

In one example, the methods described in the present invention may be implemented in a system comprising a processor and a computer readable medium that includes program code means for causing the system to carry out the steps of the methods described in the present invention. The processor may be any processor capable of carrying out the operations needed for implementation of the methods. The program code means may be any code that when implemented in the system can cause the system to carry out the steps of the methods described in the present invention. Examples of program code means include but are not limited to instructions to carry out the methods described in this patent written in a high level computer language such as C++, Java, or Fortran; instructions to carry out the methods described in the present invention written in a low level computer language such as assembly language; or instructions to carry out the methods described in the present invention in a computer executable form such as compiled and linked machine language.

Complexes formed comprising biomarker and an affinity reagent are detected by any of a number of known techniques known in the art, depending on the format of the assay and the preference of the user. For example, unlabelled affinity reagents may be detected with DNA amplification technology (e.g., for aptamers and DNA-labeled antibodies) or labeled “secondary” antibodies which bind the affinity reagent. Alternately, the affinity reagent may be labeled, and the amount of complex may be determined directly (as for dye-(fluorescent or visible), bead-, or enzyme-labeled affinity reagent) or indirectly (as for affinity reagents “tagged” with biotin, expression tags, and the like).

As will be understood by those of skill in the art, the mode of detection of the signal will depend on the detection system utilized in the assay. For example, if a radiolabeled detection reagent is utilized, the signal will be measured using a technology capable of quantitating the signal from the biological sample or of comparing the signal from the biological sample with the signal from a reference sample, such as scintillation counting, autoradiography (typically combined with scanning densitometry), and the like. If a chemiluminescent detection system is used, then the signal will typically be detected using a luminometer. Methods for detecting signal from detection systems are well known in the art and need not be further described here.

When more than one biomarker is measured, the biological sample may be divided into a number of aliquots, with separate aliquots used to measure different biomarkers (although division of the biological sample into multiple aliquots to allow multiple determinations of the levels of the biomarker in a particular sample are also contemplated). Alternately the biological sample (or an aliquot therefrom) may be tested to determine the levels of multiple biomarkers in a single reaction using an assay capable of measuring the individual levels of different biomarkers in a single assay, such as an array-type assay or assay utilizing multiplexed detection technology (e.g., an assay utilizing detection reagents labeled with different fluorescent dye markers).

It is common in the art to perform “replicate” measurements when measuring biomarkers. Replicate measurements are ordinarily obtained by splitting a sample into multiple aliquots, and separately measuring the biomarker(s) in separate reactions of the same assay system. Replicate measurements are not necessary to the methods of the invention, but many embodiments of the invention will utilize replicate testing, particularly duplicate and triplicate testing.

### Reference Values and Control Subject

The reference values used for comparison with the level from a subject for a biomarker may vary, depending on the aspect of the invention being practiced, as will be understood throughout this specification, and below. A reference value can be based on an individual sample value, such as for example, a value obtained from a sample from the subject being tested, but at an earlier point in time (e.g., a younger person in their early 20s versus same person 1-20 years later). Reference value(s) can also be based on a pool of samples, for example, value(s) obtained from samples from a pool of subjects being tested, at an earlier point in time. Reference value(s) can also be based on a pool of samples including or excluding the sample(s) to be tested. The reference value can be based on a large number of samples, such as from population of healthy subjects of the chronological age-matched group.

For age-associated disorder/disease diagnosis or risk prediction methods, a “reference value” is typically a predetermined reference level, such as an average or median of levels obtained from a population of healthy subjects that are in the chronological age group matched with the chronological age of the tested subject. As indicated earlier, in some situations, the reference samples may also be gender matched.

For age-associated disorder or disease monitoring methods (e.g., methods of monitoring disease progression in a subject diagnosed with age-associated disorder, or methods of monitoring the effect of a medical treatment or medication on a subject or a group of subjects), the reference level may be a predetermined level, such as an average or median of levels obtained from a population of healthy subjects that are in the chronological age group matched with the chronological age of the tested subject. Alternately, the reference level may be a historical reference level for the particular subject (e.g., a biomarker level that was obtained from a sample derived from the same subject, but at an earlier point in time). In some instances, the reference level may be a historical reference level for the particular groups of subjects (e.g., biomarker levels that were obtained from samples derived from the same group of subjects, but at an earlier point in time).

Healthy subjects are selected as the control subjects. Healthy subject may be used to obtain a reference level of a biomarker. A “healthy” subject or sample from a “healthy” subject or individual as used herein is the same as those commonly understood to one skilled in the art. For example, one may use methods commonly known to evaluate cognitive functions, such as learning and memory, to select control subjects as healthy subjects for diagnosis and treatment methods related to neurodegenerative diseases. In some embodiments, subjects in good health with no signs or symptom suggesting cognitive decline or neurologic disease are recruited as healthy control subjects. The subjects are evaluated based on extensive evaluations consisted of medical history, family history, physical and neurological examinations by clinicians who specialize dementia, laboratory tests, and neuropsychological assessment. For example, the examinations of neurological state of subjects, in particular the central nervous system may include any one of the followings: the assessment of consciousness, often using the Glasgow Coma Scale (EMV); mental status examination, often including the abbreviated mental test score (AMTS) or mini mental state examination (MMSE); global assessment of higher functions; estimation of intracranial pressure such as by fundoscopy. In one embodiment, Mini-Mental State Examination (MMSE) (referenced in Folstein et al., J. Psychiatr. Res 1975; 12:1289-198) was used as one of the evaluation methods to select healthy control subjects, and the healthy subjects would achieve a MMSE score equal or greater than 25. In one embodiment, the examinations of peripheral nervous system may include any one of the followings: sense of smell, visual fields and acuity, eye movements and pupils (sympathetic and parasympathetic), sensory function of face, strength of facial and shoulder girdle muscles, hearing, taste, pharyngeal movement and reflex, tongue movements, which can be tested individually (e.g. the visual acuity can be tested by a Snellen chart; A reflex hammer used testing reflexes including masseter, biceps and triceps tendon, knee tendon, ankle jerk and plantar (i.e. Babinski sign); Muscle strength often on the MRC scale 1 to 5; Muscle tone and signs of rigidity.

Age-matched populations (from which reference values may be obtained) are ideally the same chronological age as the individual being tested, but approximately age-matched populations are also acceptable. Approximately age-matched populations may be within 1, 2, 3, 4, or 5 years of the chronological age of the individual tested, or may be groups of different chronological ages which encompass the chronological age of the individual being tested.

A subject that is compared to its “chronological age matched group” is generally referring to comparing the subject with a chronological age-matched within a range of 5 to 20 years. Approximately age-matched populations may be in 2, 3, 4, 5, 6, 7, 8, 9, 10 or 15, or 20 year increments (e.g. a “5 year increment” group may serve as the source for reference values for a 62 year old subject might include 58-62 year old individuals, 59-63 year old individuals, 60-64 year old individuals, 61-65 year old individuals, or 62-66 year old individuals). In a broader definition, where there are larger gaps between different chronological age groups, for example, when there are few different chronological age groups available for reference values, and the gaps between different chronological age groups exceed the 2, 3, 4, 5, 6, 7, 8, 9, 10 or 15, or 20 year increments described herein, then the “chronological age matched group” may refer to the age group that is in closer match to the chronological age of the subject (e.g. when references values available for an older age group (e.g., 80-90 years) and a younger age group (e.g., 20-30 years), a chronological age matched group for a 51 year old may use the younger age group (20-30 years), which is closer to the chronological age of the test subject, as the reference level.

Other factors to be considered while selecting control subjects include, but not limited to, species, gender, ethnicity and so on. As described in the proteomic screening of the biomarkers in Example 1, biomarkers may or may not be shared across species. Moreover, some of the changes of biomarkers within different age groups may be gender specific. Hence in one embodiment, a reference level may be a predetermined reference level, such as an average or median of levels obtained from a population of healthy control subjects that are gender-matched with the gender of the tested subject. In one embodiment, a reference level may be a predetermined reference level, such as an average or median of levels obtained from a population of healthy control subjects that are ethnicity-matched with the ethnicity of the tested subject. In another embodiment, both chronological age and gender of the population of healthy subjects are matched with the chronological age and gender of the tested subject, respectively. In another embodiment, both chronological age and ethnicity of the population of healthy subjects are matched with the chronological age and ethnicity of the tested subject, respectively. In a further embodiment, chronological age, gender, and ethnicity of the population of healthy control subjects are all matched with the chronological age, gender, and ethnicity of the tested subject, respectively.

### Comparing Levels of Biomarkers

The process of comparing a level of biomarker from a subject and a reference level can be carried out in any convenient manner appropriate to the type of the value from the subject and reference value for the biomarker at issue. Generally, values of biomarker levels used in the methods of the invention may be quantitative values (e.g., quantitative values of concentration, such as nanograms of biomarker per milliliter of sample, or an absolute amount). Alternatively, values of biomarker level can be qualitative depending on the measurement techniques, and thus the mode of comparing a value from a subject and a reference value can vary depending on the measurement technology employed. For example, the comparison can be made by inspecting the numerical data, by inspecting representations of the data (e.g., inspecting graphical representations such as bar or line graphs). In one example, when a qualitative calorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device).

As described herein, biological fluid samples may be measured quantitatively (absolute values) or qualitatively (relative values). The respective biomarker levels for a given assessment may or may not overlap. In some embodiments, quantitative values of biomarkers in the biological fluid samples may indicate a given level of age-associated disorder or disease. For example, quantitative values of biomarkers in the biological fluid samples may indicate a given level of declining neurogenesis in aging. As shown in Example 2 and FIG. 9, increased concentrations of Eotaxin/CCL11, β2M, and/or MCP-1 in blood plasma of older age group compared to a younger age group correlate with declined neurogenesis with aging. Hence quantitative values, such as concentrations of protein biomarker, can be used to compare the concentration of a biomarker level from a subject to a reference concentration of the biomarker to diagnosis and/or monitor the progress of the age-associated disorder or disease, such as neurodegenerative diseases.

In certain embodiments, the comparison is performed to determine the magnitude of the difference between the values from a subject and reference values (e.g., comparing the “fold” or percentage difference between the value from a subject and the reference value). A fold difference that is about equal to or greater than the minimum fold difference disclosed herein suggests or indicates a diagnosis of an age-associated disorder or disease, or progression from mild disorder or disease to moderate disorder or disease, or vise versa when undergoing certain medication or medical treatment. A fold difference can be determined by measuring the absolute concentration of a protein and comparing that to the absolute value of a reference, or a fold difference can be measured by the relative difference between a reference value and a sample value, where neither value is a measure of absolute concentration, and/or where both values are measured simultaneously. For example, An ELISA measures the absolute content or concentration of a protein from which a fold change is determined in comparison to the absolute concentration of the same protein in the reference. As another example, an antibody array measures the relative concentration from which a fold change is determined. Accordingly, the magnitude of the difference between the measured value and the reference value that suggests or indicates a particular diagnosis will depend on the particular biomarker being measured to produce the measured value and the reference value used (which in turn depends on the method being practiced). Tables 5 lists an exemplary fold difference values for biomarkers indicating molecular changes between younger and older age groups.

As will be apparent to those of skill in the art, when replicate measurements are taken for the biomarker(s) tested, the value from a subjected measured that is compared with the reference value is a value that takes into account the replicate measurements. The replicate measurements may be taken into account by using either the mean or median of the measured values.

The process of comparing may be manual (such as visual inspection by the practitioner of the method) or it may be automated. For example, an assay device (such as a luminometer for measuring chemiluminescent signals) may include circuitry and software enabling it to compare a value from a subject with a reference value for a biomarker. Alternately, a separate device (e.g., a digital computer) may be used to compare the value(s) from subject(s) and the reference value(s). Automated devices for comparison may include stored reference values for the biomarker(s) being measured, or they may compare the value(s) from subject(s) with reference values that are derived from contemporaneously measured reference samples.

### Methods of Diagnosing, Treating and Monitoring Age-Associated Disorders

In one aspect, the present invention provides for methods of diagnosing an age-associated disorder in a subject, the method comprising comparing a level of at least one biomarker in a biological fluid sample from the subject to a reference level of said at least one biomarker from a population of healthy subjects without said age-associated disorder of the chronological age matched group, wherein an increased level of said biomarker from said subject compared to said reference level indicates a diagnosis of the age-associated disorder in said subject. In one embodiment, the age-associated disease is neurodegenerative disease. Exemplary neurodegenerative disease includes Alzheimer's disease, Huntington's disease, Parkinson's disease, Amyotrophic lateral sclerosis, and the like. In one embodiment, the age-associated disease is neuroinflammatory disease. The age-associated disorder may also be related to a declined cell activity, or declined tissue regeneration capacity. Exemplary cell activity includes cell proliferation, self renewal, cell differentiation, and the like. Exemplary cell includes neuronal cell and glial cell. In one embodiment, the cell is stem cell or progenitor cell, such as neural stem cell or neural progenitor cell. In one embodiment, the tissue is neural tissue. The subject to be diagnosed may be a human subject or a non-human subject. In some embodiments, the biomarkers used for diagnosis the age-associated disease or disorder may comprise at least one biomarker selected from the group consisting of Eotaxin/CCL11, β2M, MCP-1, and Haptoglobin. In some examples, the biomarker comprises at least one biomarker selected from the group consisting of Eotaxin/CCL11, β2M, and MCP-1. In some examples, the biomarker comprises β2M. In some examples, the biomarker comprises Eotaxin/CCL11. The biomarkers are obtained from biological fluid samples of the subject, which may be a peripheral biological fluid or a cerebrospinal fluid. Exemplary peripheral biological fluids include blood, serum, sputum and the like. The level of the biomarker may be determined by using a nucleic acid, such as an mRNA, or by using a protein. As an example, the level of the biomarker may be determined by using a protein detected by an immunoassay.

In some embodiment, the method of diagnosing the age-associated disorder in the subject may further comprise a step of administering a neutralizing antibody against the biomarker. For example, neutralizing antibodies against the biomarkers may be peripherally administered to the subject to treat or ameliorate the age-associated disorder. The neutralizing antibodies may include those commercially available polyclonal neutralizing antibodies against the biomarkers (e.g., neutralizing antibodies against biomarkers such as Eotaxin/CCL11, β2M, MCP-1, Haptoglobin, or the like). Monoclonal antibodies may also be used. In one embodiment humanized antibodies are used. A skilled artisan can produce suitable antibodies using routine antibody production and screening methods. Example 5 demonstrates the peripheral administration of neutralizing antibodies against β2M may inhibit the activity of the biomarker, hence promoting neural stem cell activities.

In one embodiment, provided herein is a method of diagnosing neuroinflammation in a subject, the method comprising comparing a level of at least one biomarker in a biological fluid sample from the subject to a reference level of said at least one biomarker from a population of healthy subjects without neuroinflammation of the chronological age matched group, wherein an increased level of said at least one biomarker from said subject compared to said reference level indicates a diagnosis of neuroinflammation in said subject. The method may further comprise a step of administering an anti-inflammatory agent to the subject diagnosed with neuroinflammation. In one embodiment, the anti-inflammatory agent is a non-steroidal anti-inflammatory drug (NSAID), such as aspirin, ibuprofen, or naproxen.

In some embodiments, provided herein are methods for detecting diminished cell activity in a subject, the method comprising comparing a level of at least one biomarker in a biological fluid sample from the subject to a reference level of said at least one biomarker from a population of healthy subjects having normal cell activity of the chronological age matched group, wherein an increased level of said at least one biomarker from said subject compared to said reference level indicates a diminished cell activity in said subject. Exemplary cell activity includes cell proliferation, self renewal, cell differentiation and the like. Exemplary cell includes neuronal cell and glial cell. In one embodiment, the cell is stem cell or progenitor cell, such as neural stem cell or neural progenitor cell. The subject to be diagnosed may be a human subject or a non-human subject. In some embodiments, the biomarkers used for diagnosis the age-associated disease or disorder may comprise at least one biomarker selected from the group consisting of Eotaxin/CCL11, β2M, MCP-1, and Haptoglobin. In some embodiments, the biomarker comprises at least one biomarker selected from the group consisting of Eotaxin/CCL11, β2M, and MCP-1. In some examples, the biomarker comprises β2M. In some examples, the biomarker comprises Eotaxin/CCL11. The biomarkers are obtained from biological fluid samples of the subject, which may be a peripheral biological fluid or a cerebrospinal fluid. Exemplary peripheral biological fluids include blood, serum, sputum and the like. The level of the biomarker may be determined by using a nucleic acid, such as an mRNA, or by using a protein. As an example, the level of the biomarker may be determined by using a protein detected by an immunoassay.

In some embodiments, provided herein are methods for detecting diminished tissue regeneration capacity in a subject, the method comprising comparing a level of at least one biomarker in a biological fluid sample from the subject to a reference level of said at least one biomarker from a population of healthy subjects having normal tissue regeneration activity of the chronological age matched group, wherein an increased level of said at least one tissue regeneration capacity-associated biomarker from said subject compared to said reference level indicates a diminished tissue regeneration capacity in said subject. One exemplary tissue is neural tissue. The subject to be diagnosed may be a human subject or a non-human subject. In some embodiments, the biomarkers used for diagnosis the age-associated disease or disorder may comprise at least one biomarker selected from the group consisting of Eotaxin/CCL11, β2M, MCP-1, and Haptoglobin. In some examples, the biomarker comprises at least one biomarker selected from the group consisting of Eotaxin/CCL11, β2M, and MCP-1. In some examples, the biomarker comprises β2M. In some examples, the biomarker comprises Eotaxin/CCL11. The biomarkers are obtained from biological fluid samples of the subject, which may be a peripheral biological fluid or a cerebrospinal fluid. Exemplary peripheral biological fluids include blood, serum, sputum and the like. The level of the biomarker may be determined by using a nucleic acid, such as an mRNA, or by using a protein. As an example, the level of the biomarker may be determined by using a protein detected by an immunoassay.

In another aspect, the present invention provides for methods for identifying a medical treatment or medication for a subject for promoting cell activity, increasing tissue regeneration capacity or treating an age-associated disorder or disease for a subject, the method comprising comparing at a later time point a level of at least one biomarker in a biological fluid sample from said subject exposed to said medical treatment or medication to the level of said at least one biomarker from said subject at an earlier time point, wherein a decreased level of said at least one biomarker at the later time point compared to the earlier time point indicates a suitable medical treatment or medication for promoting cell activity, increasing tissue regeneration capacity or treating said age-associated disorder for said subject.

In yet another aspect, the present invention provides for methods for identifying a medical treatment or medication for promoting cell activity, increasing tissue regeneration capacity or treating an age-associated disorder or disease for a population of subjects, the method comprising comparing at a later time point a level of at least one biomarker in biological fluid samples from a population of subjects exposed to said medical treatment or medication to the level of said at least one biomarker from said population of subjects at an earlier time point, wherein a decreased level of said at least one biomarker at the later time point compared to the earlier time point indicates a suitable medical treatment or medication for promoting stem cell or progenitor cell activity, increasing tissue regeneration capacity or treating said age-associated disorder.

The present invention thus also provides for methods of identifying a medical treatment or medication for promoting cell activity, increasing tissue regeneration capacity or treating an age-associated disorder or disease in a customized matter. Hence the medical treatment or medication may be customized to target a particular subject or a specific population of subjects. The methods provided herein allow for identifying a customized medical treatment or medication that may be more effective to the targeted subject (personalized medicine or personalized medical treatment) than the general medical treatment or medication to the general population. Moreover, certain profile factors may affect the age-specific pattern of biomarkers, such as species, gender, ethnicity, and so on. For example, biomarkers may present different changing patterns from subjects with different genders. In this regard, customized medical treatment or medication targeting particularly a population of subjects sharing same profile factors may be more effective to the targeted population of subjects than the general medical treatment or medication targeting the general population. As an example, a customized medical treatment or medication targeting particularly a population of female subjects may be more effective to female subjects than the general medical treatment or medication targeting a population of subjects with mixed genders.

Another aspect of the present invention relates to methods of monitoring the effect of a medical treatment or a medication on a subject for promoting cell activity, increasing tissue regeneration capacity or treating an age-associated disorder, the method comprising comparing at a later time point a level of at least one biomarker in a biological fluid sample from said subject exposed to said medical treatment or medication to the level of said at least one biomarker from said subject at an earlier time point, wherein a decreased level of said at least one biomarker at the later time point compared to the earlier time point indicates an effective medical treatment or medication on said subject for promoting cell activity, increasing tissue regeneration capacity or treating said age-associated disorder.

In the methods of identifying medical treatment or medications targeting a subject or a population of subject and methods of monitoring the effect of these medical treatment or medications, an exemplary age-associated disease is neurodegenerative disease. Exemplary neurodegenerative disease includes Alzheimer's disease, Huntington's disease, Parkinson's disease, Amyotrophic lateral sclerosis, and the like. In one embodiment, the age-associated disease is neuroinflammatory disease. The age-associated disorder may also be related to a declined cell activity, or declined tissue regeneration capacity. Exemplary cell activity includes cell proliferation, self renewal, cell differentiation, and the like. Exemplary cell includes neuronal cell and glial cell. In one embodiment, the cell is stem cell or progenitor cell, such as neural stem cell or neural progenitor cell. In one embodiment, the tissue is neural tissue. The subject to be treated or monitored may be a human subject or a non-human subject. In some embodiments, the biomarkers used for identifying the treatment or monitoring the age-associated disease or disorder may comprise at least one biomarker selected from the group consisting of Eotaxin/CCL11, β2M, MCP-1, and Haptoglobin. In some examples, the biomarker comprises at least one biomarker selected from the group consisting of Eotaxin/CCL11, β2M, and MCP-1. In some examples, the biomarker comprises β2M. In some examples, the biomarker comprises Eotaxin/CCL11. The biomarkers are obtained from biological fluid samples of the subject, which may be a peripheral biological fluid or a cerebrospinal fluid. Exemplary peripheral biological fluids include blood, serum, sputum and the like. The level of the biomarker may be determined by using a nucleic acid, such as an mRNA, or by using a protein. As an example, the level of the biomarker may be determined by using a protein detected by an immunoassay.

### Screening Agents for Modulating Biomarker Activity

Another aspect of the present invention provides for methods of screening for candidate agents for the treatment of age-associated disorders or diseases by identifying candidate agents for activity in modulating age-associated disorders/diseases biomarkers. The screening may be performed with a screening assay either in vitro and/or in vivo. Candidate agents identified in the screening methods described herein may be useful as therapeutic agents for the treatment of age-associated disorder or diseases, as those described herein.

Thus some embodiments of the present invention provides for methods of identifying a candidate agent for modulating the activity or expression of a biomarker selected from the group consisting of Eotaxin/CCL11, β2M, MCP-1 and Haptoglobin, the method comprising contacting said candidate agent in an assay; detecting the expression or activity of said biomarker; and comparing the expression or activity of said biomarker to a reference level of said biomarker, wherein an decreased expression or activity of said biomarker indicates an inhibition of the expression or activity of said biomarker by said candidate agent, and wherein an increased expression or activity of said biomarker indicates a promotion of the expression or activity of said biomarker by said candidate agent.

The screening methods of the invention utilize the biomarkers described herein as the targets, and prospective agents are tested for activity in modulating a target in an assay system. As understood by those of skill in the art, the mode of testing for modulation activity will depend on the biomarker and the form of the target used (e.g., protein or gene). A wide variety of suitable assays are known in the art. When the biomarker protein itself is the target, prospective agents are tested for activity in modulating expression levels or activity of the protein itself. Modulation of expression levels of a biomarker can be accomplished by, for example, increasing or reducing half-life of the biomarker protein. Modulation of activity of a biomarker can be accomplished by increasing or reducing the availability of the biomarker to bind to its cognate receptor(s) or ligand(s). When a biomarker polynucleotide is the target, the prospective agent is tested for activity in modulating synthesis of the biomarker, for example, by measuring either mRNA transcribed from the gene (transcriptional modulation) or by measuring protein produced as a consequence of such transcription (translational modulation). As understood by those in the art, many assay formats will utilize a modified form of the biomarker gene where a heterologous sequence (e.g., encoding an expression marker such as an enzyme or an expression tag such as oligo-histidine or a sequence derived from another protein, such as myc) is fused to (or even replaces) the sequence encoding the biomarker protein. Such heterologous sequence(s) allow for convenient detection of levels of protein transcribed from the target.

Prospective agents for use in the screening methods of the invention may be chemical compounds and/or complexes of any sort, including both organic and inorganic molecules (and complexes thereof). Screening assays may be in any format known in the art, including cell-free in vitro assays, cell culture assays, organ culture assays, and in vivo assays (i.e., assays utilizing animal models).

Accordingly, in some embodiments, the screening assay is in vitro assay. In a further embodiment, the screening assay is a cell-free assay. Each prospective agent is incubated with the target in a cell-free environment, and modulation of expression or activity of the biomarker is measured. Cell-free environments useful in the screening methods of the invention include cell lysates (particularly useful when the target is a biomarker gene) and biological fluids such as whole blood or fractionated fluids derived therefrom such as plasma and serum (particularly useful when the biomarker protein is the target). When the target is a biomarker gene, the modulation measured may be modulation of transcription or translation. When the target is the biomarker protein, the modulation may of the half-life of the protein or of the availability of the biomarker protein to bind to its cognate receptor or ligand.

In other embodiments, the screening assay is a cell-based assay. Each prospective agent is incubated with cultured cells, and modulation of the expression or activity of the target biomarker is measured. In certain embodiments, the cultured cells are astrocytes, neuronal cells (such as hippocampal neurons), fibroblasts, or glial cells. When the target is a biomarker gene, transcriptional or translational modulation may be measured. When the target is the biomarker protein, the biomarker protein is also added to the assay mixture, and modulation of the half-life of the protein or of the availability of the biomarker protein to bind to its cognate receptor or ligand is measured.

In some other embodiments, the screening assay is an organ culture-based assay. In this format, each prospective agent is incubated with either a whole organ or a portion of an organ (such as a portion of brain tissue, such as a brain slice) derived from a non-human animal and modulation of the expression or activity of the target biomarker is measured. When the target is a biomarker gene, transcriptional or translational modulation may be measured. When the target is the biomarker protein, the biomarker protein is also added to the assay mixture, and modulation of the half-life of the protein or of the availability of the biomarker protein to bind to its cognate receptor is measured.

In yet other embodiments, the screening assay is in vivo assays. In this format, each prospective agent is administered to a non-human animal and modulation of the expression or activity of the target biomarker is measured. When the target is a biomarker gene, transcriptional or translational modulation may be measured. When the target is the biomarker protein, modulation of the half-life of the target biomarker or of the availability of the biomarker protein to bind to its cognate receptor or ligand is measured. A wide variety of methods are known in the art for measuring modulation of transcription, translation, protein half-life, protein availability, and other aspects which can be measured. In view of the common knowledge of these techniques, they need not be further described here.

Another aspect of the present invention provides for methods of screening for receptors or ligands that can bind to the age-associated disorders/diseases biomarkers. By utilizing antagonists to the identified receptors to the biomarkers, activity of the biomarkers can be modulated, and hence eventually achieving the treatment of age-associated disorders or diseases. Thus some embodiments provides for methods of identifying a receptor for a biomarker selected from the group consisting of Eotaxin/CCL11, β2M, MCP-1 and Haptoglobin, the method comprising contacting a cell transfected with a nucleic acid encoding a candidate receptor with the biomarkers under conditions suitable for binding, and detecting specific binding of the biomarkers to the candidate receptor, wherein binding to the candidate receptor is indicative of a receptor for the biomarker. An exemplary embodiment of identifying receptors for the biomarkers β2M is described in Example 3. The method exemplified can be also used with other biomarkers disclosed herein. In one embodiment, provided are methods of inhibiting activity of or expression of a biomarker selected from the group consisting of Eotaxin/CCL11, β2M, MCP-1 and Haptoglobin. The method comprising contacting a cell or a tissue expressing the biomarker with an antagonist targeting the candidate receptor identified using the method described above.

### Devices and Kits

Provided herein are also kits and devices for carrying out any of the methods described herein. Kits of the present invention may comprise at least one reagent specific to at least one biomarker, and may further include instructions for carrying out a method described herein.

The at least one biomarker includes any one of the biomarkers listed herein including those listed in Table 1, Table 2 and Table 3. An embodiment includes those described in the section of “Age-associated biomarkers.”

In some embodiments, the present invention provides for a kit comprising at least one reagent specific to at least one age-associated biomarker, said at least one biomarker selected from the group consisting of Eotaxin/CCL11, β2M, MCP-1, and Haptoglobin; and instructions for carrying out any of the method described above in the present invention. In some embodiments, the kit comprises any one, two, three or four of the biomarkers Eotaxin/CCL11, β2M, MCP-1, and Haptoglobin.

In some embodiments, the kit comprises at least two or more different biomarker-specific affinity reagents, where each reagent is specific for a different biomarker. In some embodiments, the reagent(s) specific for a biomarker is an affinity reagent.

Kits comprising a single reagent specific for a biomarker may have the reagent enclosed in a container (e.g., a vial, ampoule, or other suitable storage container). Alternatively, the reagent may be bound to a substrate (e.g., an inner surface of an assay reaction vessel) are also contemplated. Likewise, kits including more than one reagent may also have the reagents in containers (separately or in a mixture) or may have the reagents bound to a substrate.

Thus, in some embodiments, the kit further comprises at least one solid support wherein the reagent specific to at least one age-associated biomarker is deposited on the support. In some examples, the solid support is in the format of a dipstick, a test strip, a latex bead, a microsphere or a multi-well plate.

In some embodiments, the biomarker-specific reagent(s) may be labeled with a detectable marker (such as a fluorescent dye or a detectable enzyme), or may be modified to facilitate detection (e.g., biotinylated to allow for detection with an avidin- or streptavidin-based detection system). In other embodiments, the biomarker-specific reagent may not be directly labeled or modified.

In certain embodiments, kits may also include one or more agents for detection of bound biomarker specific reagent. Detection agents and detection systems are those known in the art. For example, detection agents may include antibodies specific for the biomarker-specific reagent (e.g., secondary antibodies), primers for amplification of a biomarker-specific reagent that is nucleotide based (e.g., aptamer) or of a nucleotide ‘tag’ attached to the biomarker-specific reagent, avidin- or streptavidin-conjugates for detection of biotin-modified biomarker-specific reagent(s), and the like.

A modified substrate or other system for capture of biomarkers may also be included in the kits of the invention, particularly when the kit is designed for use in a sandwich-format assay. The capture system may be any capture system useful in a biomarker assay system, as known in the art, such as a multi-well plate coated with a biomarker specific reagent, beads coated with a biomarker-specific reagent, and the like.

In certain embodiments, kits for use in the methods disclosed herein include the reagents in the form of an array. The array includes at least two different reagents specific for biomarkers (each reagent specific for a different biomarker) bound to a substrate in a predetermined pattern (e.g., a grid). The localization of the different biomarker-specific reagents (the “capture reagents”) allows measurement of levels of a number of different biomarkers in the same reaction. Kits including the reagents in array form may be in a sandwich format, so such kits may also comprise detection reagents. Generally, the kit will include different detection reagents, each detection reagent specific to a different biomarker. The detection reagents in such embodiments are normally reagents specific for the same biomarkers as the reagents bound to the substrate (although the detection reagents typically bind to a different portion or site on the biomarker target than the substrate-bound reagents), and are generally affinity-type detection reagents. As with detection reagents for any other format assay, the detection reagents may be modified with a detectable moiety, modified to allow binding of a separate detectable moiety, or be unmodified. Array-type kits including detection reagents that are either unmodified or modified to allow binding of a separate detectable moiety may also contain additional detectable moieties (e.g., detectable moieties which bind to the detection reagent, such as labeled antibodies which bind unmodified detection reagents or streptavidin modified with a detectable moiety for detecting biotin-modified detection reagents).

The instructions in the kit relating to the use of the kit for carrying out the invention generally describe how the contents of the kit are used to carry out the methods of the invention. Instructions may include information as sample requirements (e.g., form, pre-assay processing, and size), steps necessary to measure the biomarker(s), interpretation of results, and the like. Instructions supplied in the kits may include written instructions on a label or package insert (e.g., a paper sheet included in the kit), or machine-readable instructions (e.g., instructions carried on a magnetic or optical storage disk). In certain embodiments, machine-readable instructions comprise software for a programmable digital computer for comparing the measured values obtained using the reagents included in the kit.

In some embodiments, kits may also comprise a set of reference values for at least one biomarker from a population of people from different chronological age groups. These reference values may be used to compare the level of biomarkers from the tested sample to diagnosis the disease or monitor the disease progression. The biomarkers referred to in these embodiments may be any biomarker disclosed in the present invention. In one example, the biomarkers include any one, two, three or four of Eotaxin/CCL11, β2M, MCP-1, and Haptoglobin.

In another aspect, the present invention provides for a device comprising a measuring assembly yielding detectable signal from an assay indicating the presence or level of an age-associated biomarker from the biological fluid sample of an individual; and an output assembly for displaying an output content for the user. The device may further comprise a sample collection unit. The device may also comprise a storage assembly configured to store data output from the measuring assembly; and a comparison assembly adapted to compare the data stored on the storage assembly with reference data, and to provide a retrieved data as the output content. Alternatively, the device may comprise a communication assembly for transmitting data from the measuring assembly to an external device to compare with reference data, and to transmitting a retrieved data back from the external device to the device as the output content.

In one embodiment, the device may be a handheld device, for example, a home use device.

In one embodiment, the data are stored in an external device, which may serve as a bioinformatics server, i.e., to store data bases including all the reference data. In this regard, data read from the measuring assembly may be transmitted to the external data through the communication assembly, and a retrieved data may be transmitted back from the external device to the device as the output content. Further, the transmitted data from measured assembly may be analyzed and the analyzed result may be transmitted back from the external device to the device as the output content.

In yet another aspect, the present invention provides for a system comprising a determination module configured to receive and output a measuring information indicating the presence or level of an age-associated biomarker from the biological fluid sample of an individual; a storage assembly configured to store output information from the determination module; a comparison module adapted to compare the data stored on the storage module with reference data, and to provide a comparison content; and an output module for displaying the comparison content for the user.

### Treatment of Age-Associated Diseases

Pharmaceutical compositions comprising antagonists to the biomarkers or their receptors as described that are useful in treatment of the age-associated diseases can be formulated as is well known in the art.

Such formulations can be administered to the subjects using systemic or local administration. For example, one can administer the pharmaceutical compositions directly into the brain, intracranially. Alternatively, one can administer the pharmaceutical compositions systemically, such as intravenously, orally, intramuscularly, intraperitoneally, or subcutaneously. Nasal sprays or inhaled aerosols are also contemplated.

### Additional Applications of the Methods of the Invention

Additional useful applications of the methods as described herein include, e.g., screening of donated plasma. Thus, one can determine the amount of any one of the age-associated markers in a plasma from a donor, or in a pooled plasma sample from multiple donors and if the amount of the marker protein is too high, e.g., at a level which is associated with moderate to severe cognitive impairment, one can either discard the plasma sample. Alternatively, if such plasma sample is used, one can determine that when used, it should be used together with a neutralizing antibody or neutralizing RNA-interfering agent targeting the specific protein. For example, if the amount of CCL2/MCP-1 is determined to be, for example, two fold or more or four fold or more than that of a reference value from, e.g., healthy 20-45 yr old humans, the donated plasma should not be used or should be used together with a neutralizing antibody or RNA interfering agent against CCL2/MCP-1.

### EXAMPLES

### Methods

Summary of Methods:

C57BL/6 (Jackson Laboratory), C57BL/6 aged mice (National Institutes of Aging), Dcx-Luc26, and C57BL/6J-Act-GFP (Jackson Laboratory). For all in vivo pharmacological and behavioral studies young (2-3 months) wild type C57BL/6 male mice were used. All animal use was in accordance with institutional guidelines approved by the VA Palo Alto Committee on Animal Research. Parabiosis surgery followed previously described procedures (Monje, M. L., Toda, H., & Palmer, T. D., Science 302 (5651), 1760-1765 (2003)) with the addition that peritonea between animals were surgically connected. Immunohistochemistry was performed on free-floating sections following standard published techniques (Luo, J. et al., J Clin Invest 117 (11), 3306-3315 (2007)). Hippocampal slice extracellular electrophysiology was performed as previously described (Xie, X. & Smart, T. G., Pflugers Arch 427 (5-6), 481-486 (1994)). Spatial learning and memory was assayed with the radial arm water maze (RAWM) paradigm as previously published (Alamed, J., et al. Nat Protoc 1 (4), 1671-1679 (2006)). Mouse plasma was prepared by centrifugation and systemically administered via intravenous injections. Relative plasma concentrations of cytokines and signaling molecules in mice and humans were measured using antibody-based multiplex immunoassays at Rules Based Medicine, Inc. Human plasma and CSF samples were obtained from academic centers and informed consent was obtained from human subjects according to the institutional review board guidelines at the respective centers. Recombinant murine CCL11 (R&D Systems), rat IgG2a neutralizing antibody against mouse CCL11 (R&D Systems), and control rat IgG2a (R&D Systems) were administered either systemically by intraperitoneal injection or locally by unilateral stereotaxic injection into the dentate gyrus of the hippocampus. Statistical analysis was performed with Prism 5.0 software (GraphPad Software). Plasma protein correlations in the aging samples were analyzed with the Significance Analysis of Microarray software (SAM 3.00 algorithm).

Mice.

The following mouse lines were used: C57BL/6 (The Jackson Laboratory), C57BL/6 aged mice (National Institutes of Aging), Dcx-Luc mice (Couillard-Despres, S. et al., In vivo optical imaging of neurogenesis: watching new neurons in the intact brain. Mol Imaging 7 (1), 28-34 (2008)), and C57BL/6J-Act-GFP (Jackson Laboratory). For all in vivo pharmacological and behavioral studies young (2-3 months) wild type C57BL/6 male mice were used. Mice were housed under specific pathogen-free conditions under a 12 h light-dark cycle and all animal handling and use was in accordance with institutional guidelines approved by the VA Palo Alto Committee on Animal Research. All experiments were done in a randomized and blinded fashion.

Immunohistochemistry.

Tissue processing and immunohistochemistry was performed on free-floating sections following standard published techniques (Luo, J. et al., Glia-dependent TGF-beta signaling, acting independently of the TH17 pathway, is critical for initiation of murine autoimmune encephalomyelitis. J Clin Invest 117 (11), 3306-3315 (2007)). Briefly, mice were anesthetized with 400 mg/kg chloral hydrate (Sigma-Aldrich) and transcardially perfused with 0.9% saline Brains were removed and fixed in phosphate-buffered 4% paraformaldehyde, pH 7.4, at 4° C. for 48 h before they were sunk through 30% sucrose for cryoprotection. Brains were then sectioned coronally at 40 μm with a cryomicrotome (Leica Camera, Inc.) and stored in cryoprotective medium. Primary antibodies were: goat anti-Dcx (1:500; Santa Cruz Biotechnology), rat anti-BrdU (1:5000, Accurate Chemical and Scientific Corp.), goat anti-Sox2 (1:200; Santa Cruz), mouse anti-NeuN (1:1000, Chemicon), mouse anti-GFAP (1:1500, DAKO), and mouse anti-CD68 (1:50, Serotec). After overnight incubation, primary antibody staining was revealed using biotinylated secondary antibodies and the ABC kit (Vector) with Diaminobenzidine (DAB, Sigma-Aldrich) or fluorescence conjugated secondary antibodies. For BrdU labeling, brain sections were pre-treated with 2N HCl at 37° C. for 30 min before incubation with primary antibody. For double-label immunofluorescence of BrdU/NeuN or BrdU/GFAP, sections were incubated overnight with rat anti-BrdU, rinsed, and incubated for 1 hr with donkey anti-rat antibody (2.5 μg/ml, Vector) before they were stained with mouse anti-NeuN antibody.

To estimate the total number of Dcx or Sox2 positive cells per DG immunopositive cells in the granule cell and subgranular cell layer of the DG were counted in every sixth coronal hemibrain section through the hippocampus and multiplied by 12.

BrdU Administration and Quantification of BrdU-Positive Cells.

50 mg/kg of BrdU was injected intraperitoneally into mice once a day for 6 days, and mice were sacrificed 28 days later or injected daily for 3 days before sacrifice. To estimate the total number of BrdU-positive cells in the brain, we performed DAB staining for BrdU on every sixth hemibrain section. The number of BrdU+ cells in the granule cell and subgranular cell layer of the DG were counted and multiplied by 12 to estimate the total number of BrdU-positive cells in the entire DG. To determine the fate of dividing cells a total of 200 BrdU-positive cells across 4-6 sections per mouse were analyzed by confocal microscopy for co-expression with NeuN and GFAP. The number of double-positive cells was expressed as a percentage of BrdU-positive cells.

Parabiosis and Flow Cytometry.

Parabiosis surgery followed previously described procedures (Conboy, I. M. et al., Rejuvenation of aged progenitor cells by exposure to a young systemic environment. Nature 433 (7027), 760-764 (2005)). Pairs of mice were anesthetized and prepared for surgery. Mirror-image incisions at the left and right flanks, respectively, were made through the skin. Shorter incisions were made through the abdominal wall. The peritoneal openings of the adjacent parabionts were sutured together. Elbow and knee joints from each parabiont were sutured together and the skin of each mouse was stapled (9 mm Autoclip, Clay Adams) to the skin of the adjacent parabiont. Each mouse was injected subcutaneously with Baytril antibiotic and Buprenex as directed for pain and monitored during recovery. Flow cytometric analysis was done on fixed and permeabilized blood plasma cells from GFP and non-GFP parabionts. Approximately 40-60% of cells in the blood of either parabiont were GFP-positive two weeks after parabiosis surgery. We observed 70-80% survival rate in parabionts five weeks post parabiosis surgery.

Extracellular Electrophysiology.

Acute hippocampal slices (400 μm thick) were prepared from unpaired and young parabionts. Slices were maintained in artificial cerebrospinal fluid (ACSF) continuously oxygenated with 5% CO2/95% O2. ACSF composition was as follows: (in mM): NaCl 124.0; KCl 2.5; KH2PO4 1.2; CaCl2 2.4; MgSO4 1.3; NaHCO3 26.0; glucose 10.0 (pH 7.4). Recordings were performed with an Axopatch-2B amplifier and pClamp 10.2 software (Axon Instruments). Submerged slices were continuously perfused with oxygenated ACSF at a flow rate of 2 ml/min from a reservoir by gravity feeding. Field potential (population spikes and EPSP) was recorded using glass microelectrodes filled with ACSF (resistance: 4-8 MΩ). Biphasic current pulses (0.2 ms duration for one phase, 0.4 ms in total) were delivered in 10 s intervals through a concentric bipolar stimulating electrode (FHC, Inc.). No obvious synaptic depression or facilitation was observed with this frequency stimulation. To record field population spikes in the dentate gyrus, the recording electrode was placed in the lateral or medial side of the dorsal part of the dentate gyrus. The stimulating electrode was placed right above the hippocampal fissure to stimulate the perforant pathway fibers. Signals were filtered at 1 KHz and digitized at 10 KHz. Tetanic stimulation consisted of 2 trains of 100 pulses (0.4 ms pulse duration, 100 Hz) delivered with an inter-train interval of 5 seconds. The amplitude of population spike was measured from the initial phase of the negative wave. Up to five consecutive traces were averaged for each measurement. LTP was calculated as mean percentage change in the amplitude of the population spike following high frequency stimulation relative to its basal amplitude.

Behavioral Assay.

Spatial learning and memory was assessed using the radial arm water maze (RAWM) paradigm following the exact protocol described by Alamed et al. in Nature Protocols (Alamed, J., Wilcock, D. M., Diamond, D. M., Gordon, M. N., & Morgan, D., Two-day radial-arm water maze learning and memory task; robust resolution of amyloid-related memory deficits in transgenic mice. Nat Protoc 1 (4), 1671-1679 (2006)). Behavioral analysis was performed for normal aging mice at young (2-3 months) and old (18 months) ages, for young adult mice (2-3 months) injected intravenously with plasma isolated from young (3-4 months) and old (18-20 months) mice every three days for 24 days, and for young adult mice (3-4 months) injected intraperitoneally with murine recombinant CCL11 and PBS vehicle for five weeks. The goal arm location containing a platform remains constant throughout the training and testing phase, while the start arm is changed during each trial. On day one during the training phase, mice are trained for 15 trails, with trials alternating between a visible and hidden platform. On day two during the testing phase, mice are tested for 15 trials with a hidden platform. Entry into an incorrect arm is scored as an error, and errors are averaged over training blocks (three consecutive trials). All studies were done by an investigator that was blinded to the age or treatment of mice.

Plasma Collection and Proteomic Analysis.

Mouse blood was collected into EDTA coated tubes via tail vein bleed, mandibular vein bleed, or intracardial bleed at time of sacrifice. EDTA plasma was generated by centrifugation of freshly collected blood and aliquots were stored at −80° C. until use. Human plasma and CSF samples were obtained from academic centers and subjects were chosen based on standardized inclusion and exclusion criteria as previously described (Zhang, J. et al., CSF multianalyte profile distinguishes Alzheimer and Parkinson diseases. Am J Clin Pathol 129 (4), 526-529 (2008); Li, G. et al., Cerebrospinal fluid concentration of brain-derived neurotrophic factor and cognitive function in non-demented subjects. PLoS One 4 (5), e5424 (2009)) and outlined below. Mouse and human plasma samples were sent to Rules Based Medicine Inc., a fee-for-service provider, where the relative plasma concentrations of cytokines and signaling molecules were measured using standard antibody-based multiplex immunoassays in a blinded fashion. All assays were developed and validated to Clinical Laboratory Standards Institute (formerly NCCLS) guidelines based upon the principles of immunoassay as described by the manufacturers.

| Aging subject inclusion criteria | Aging subject exclusion criteria |
|---|---|
| Age: Subject meets age cutoffs for entry to the | Vision and/or hearing too impaired (even with |
| specific diagnostic group. | correction) to allow compliance with psychometric |
| Informant: Presence of an informant for all | testing |
| subjects. | Medical problems: unstable, poorly controlled, or |
| General health: good enough to complete study | severe medical problems or diseases. |
| visits. | Cancer in the past 12 months (excludes squamous |
| Body Mass Index (BMI): 18-34 | CA of the skin or stage 1 prostate CA). |
| Stable medications for 4 weeks before the visit to | Contraindications to lumbar puncture: Bleeding |
| draw blood or CSF. | disorder, use of Coumadin, heparin or similar |
| Permitted medications include: AChE-inhibitors, | anticoagulant, platelets <100,000; deformity or |
| Memantine, HRT (estrogen +/− progesterone, | surgery affecting lumbosacral spine which is severe |
| Lupron), Thyroid hormone, Antidepressants, | enough to make lumbar puncture difficult, |
| statins. | cutaneous sepsis at lumbosacral region. |
| Normal basic laboratory tests: BUN, creatinine | Neurological disorders: neurodegenerative diseases |
| (will allow creatinine up to 1.5), B12, TSH. | such as Alzheimer's Disease, Parkinson's Disease, |
| MMSE >27/30 (exemptions if low education and | CJD, FTD, PSP; stroke in past 12 months or severe |
| control status established by detailed evaluation) | enough residual effects of earlier stroke to impair |
| Memory performance on logical Memory within | neurological or cognitive function; Multiple |
| normal limits. | sclerosis; epilepsy |
| CDR = 0 | Psychiatric disorders: schizophrenia, bipolar |
| Neurological exam is normal, i.e. no evidence of | affective disorder |
| stroke, Parkinsonism or major abnormalities. | Active/uncontrolled depression: by history or GDS |
| | score |
| | Drug or alcohol abuse in past 2 years |
| | Exclusionary medications (in 4 weeks before visit |
| | to draw blood or CSF) |
| | Neuroleptics/atypical antipsychotics |
| | Anti-Parkinson's Disease medications (L-dopa, |
| | dopamine agonists) |
| | CNS stimulants: modafinil, Ritalin |
| | Antiepileptic drugs (exceptions for Neurontin or |
| | similar newer AEDs given for pain control) |
| | Insulin treatment |
| | Cortisone (oral prohibited - topical or inhaler use |
| | allowed), anti-immune drugs (e.g. methotrexate, |
| | cytoxan, IVIg, tacrolimus, cyclosporine) or |
| | antineoplastic drugs |
| | Anti-HIV medications |

CCL11, MSCF, Antibody, or Plasma Administration.

Carrier free recombinant murine CCL11 dissolved in PBS (10 μg/kg; R&D Systems), carrier free recombinant MCSF dissolved in PBS (10 μg/kg; Biogen), rat IgG2a neutralizing antibody against mouse CCL11 (50 μg/ml; R&D Systems, Clone: 42285), and isotype matched control rat IgG2a recommended by the manufacturer (R&D Systems, Clone: 54447) were administered systemically via intraperitoneal injection over ten days on day 1, 4, 7, and 10. The same reagents (0.50 μl; 0.1 μg/ul) were also administered stereotaxically into the DG of the hippocampus in some experiments (coordinates from bregma: A=−2.0 mm and L=−1.8 mm, from brain surface: H=−2.0 mm). Pooled mouse serum or plasma was collected from 2-3-month-old (young) mice and 18-20-month-old (aged) mice by intracardial bleed at time of sacrifice. Serum was prepared from clotted blood collected without anticoagulants; plasma was prepared from blood collected with EDTA followed by centrifugation. Aliquots were stored at −80° C. until use. Prior to administration plasma was dialyzed in PBS to remove EDTA. Young adult mice were systemically treated with plasma (100 μl) isolated from young or aged mice via intravenous injections every three days for ten days.

In Vivo Bioluminescence Imaging.

Bioluminescence was detected with the In Vivo Imaging System (IVIS Spectrum; Caliper Life Science). Mice were injected intraperitoneally with 150 mg/kg D-luciferin (Xenogen) 10 minutes before imaging and anesthetized with isofluorane during imaging. Photons emitted from living mice were acquired as photons/s/cm2/steridan (sr) using LIVINGIMAGE software (version 3.5, Caliper) and integrated over 5 minutes. For quantification a region of interest was manually selected and kept constant for all experiments.

Cell Culture Assays.

Mouse neural progenitor cells were isolated from C57BL/6 mice as previously described (Renault, V. M. et al., FoxO3 regulates neural stem cell homeostasis. Cell Stem Cell 5 (5), 527-539 (2009)). Brains from postnatal animals (1 day-old) were dissected to remove olfactory bulbs, cerebellum and brainstem. After removing superficial blood vessels forebrains were finely minced, digested for 30 minutes at 37° C. in DMEM media containing 2.5 U/ml Papain (Worthington Biochemicals), 1 U/ml Dispase II (Boeringher Mannheim), and 250 U/ml DNase I (Worthington Biochemicals) and mechanically dissociated. NSC/progenitors were purified using a 65% Percoll gradient and plated on uncoated tissue culture dishes at a density of 105 cells/cm2. NPCs were cultured under standard conditions in NeuroBasal A medium supplemented with penicillin (100 U/ml), streptomycin (100 mg/ml), 2 mM L-glutamine, serum-free B27 supplement without vitamin A (Sigma-Aldrich), bFGF (20 ng/ml) and EGF (20 ng/ml). Carrier free forms of murine recombinant CCL2 (100 ng/ml; R&D Systemcs), murine recombinant CCL11 (100 ng/ml, R&D Systemcs), rat IgG2b neutralizing antibody against mouse CCL2 (10 ug/ml; R&D Systems, Clone: 123616), control rat IgG2b (10 μg/ml; R&D Systems, Clone: 141945), goat IgG neutralizing antibody against mouse CCL11 (10 μg/ml; R&D Systems), and control goat IgG (10 μg/ml; R&D Systems) were dissolved in PBS and added to cell cultures under self-renewal conditions every other day following cell plating.

Human NTERA cells (Renault, V. M. et al., FoxO3 regulates neural stem cell homeostasis. Cell Stem Cell 5 (5), 527-539 (2009)) expressing eGFP under the doublecortin promoter were cultured under standard self-renewal and differentiation conditions (Couillard-Despres, S. et al., Human in vitro reporter model of neuronal development and early differentiation processes. BMC Neurosci 9, 31 (2008); Buckwalter, M. S. et al., Chronically increased transforming growth factor-beta1 strongly inhibits hippocampal neurogenesis in aged mice. Am J Pathol 169 (1), 154-164 (2006)). Carrier free forms of human recombinant CCL2 (100 ng/ml, R&D Systems), human recombinant CCL11 (100 ng/ml, R&D Systems), mouse IgG1 neutralizing antibody against human CCL11 (25 μg/ml; R&D Systems, Clone: 43911) and control mouse IgG1 (25 μg/ml; R&D Systems) were added to cell cultures under differentiation conditions every other day following cell plating.

Data and Statistical Analysis.

Data are expressed as mean±SEM. Statistical analysis was performed with Prism 5.0 software (GraphPad Software). Means between two groups were compared with two-tailed, unpaired Student's t test. Comparisons of means from multiple groups with each other or against one control group were analyzed with 1-way ANOVA and Tukey-Kramer's or Dunnett's post hoc tests, respectively. Plasma protein correlations in the aging samples were analyzed with the Significance Analysis of Microarray software (SAM 3.00 algorithm, see, e.g., R. Hughey and A. Krogh, Technical Report UCSC-CRL-95-7, University of California, Santa Cruz, Calif., January 1995. (Last update prior to filing of application No. 61/298,998), The SAM documentation.). Unsupervised cluster analysis was performed using Gene Cluster 3.0 software and node maps were produced using Java TreeView 1.0.13 software.

### Example 1

### Proteomic Screening of Age-Associated Biomarkers and the Use of these Biomarkers to Assess the Age

Proteomic Screening of Biomarkers in Human Plasma and the Use of these Biomarkers to Assess the Age of Human

Healthy control subjects in good health with no signs or symptoms suggesting cognitive decline or neurologic disease were recruited for multicentre studies that aim to identify molecular biomarkers for healthy aging in blood and CSF. Human subjects divisions at each institution approved this study. Following informed consent, all subjects underwent extensive evaluations including medical history, family history, physical and neurologic examinations by clinicians specializing in dementia, laboratory tests, and neuropsychological assessment.

Human plasma and CSF samples were obtained from academic centers courtesy of Christopher M. Clark, Douglas R. Galasko, Jeffrey A. Kaye, Ge Li, Elaine R. Peskind, and Joseph F. Quinn. Shortly after venous blood draw, EDTA plasma was isolated by spinning the vacutainer at 1000 g for 10 minutes at 4° C. and followed by immediate aliquoting of the plasma and freezing at −80° C. Aliquots were not thawed until analysis. Detections of plasma concentrations of biomarkers were performed at Rules Based Medicine (Austin, Tex.) with an established and proprietary antibody-based multiplexed Luminex assay. M-CSF was detected by a specific QUANTIKINE® ELISA (R&D System, Inc., Minneapolis, Minn.) following the company's instructions.

We measured the concentrations of 89 secreted intercellular signaling proteins in 188 archived plasma samples from healthy aging individuals (93 women, 95 men, age range 21-88 years, median age 61 years, mean age 56.5 years) with flow cytometry-based Luminex technology. One additional factor was measured by ELISA. Out of these 90 markers, 76 proteins were detectable in most samples (i.e., 13 proteins were detectable in fewer than 90% of the samples and were considered herein as undetectable). Comparing biomarker levels in plasma of different age groups, 44 discriminatory biomarkers were revealed that have significantly changed expression levels in the older group (75-88 years) in comparison to the younger group (20-44 years), as shown in Table 1. For 29 proteins, expression levels were at least 1.2 fold higher on average in each older individual in the older group than those individuals in the younger group, and none were significantly decreased. Some of these changes are sex specific.

To identify plasma signaling proteins that best characterize age, log₂-normalized data were analyzed with the statistical method Elastic net (Enet [87]). This method is a regularization and variable selection method that identifies significant correlations between variables of interest in a large number of observations (i.e. age or relative proliferation correlated with results of a gene or proteomic microarrays). An internal correction algorithm and a 10-fold cross validation step assess and minimize classification error. Cluster analysis of the top ten predictor proteins in female individuals produced a distinct separation of samples by age. A similar node map was obtained after cluster analysis of these top ten markers in the samples from male individuals. These top ten biomarkers include Adiponectin/Acrp30; Apolipoprotein A-1 (ApoA1); β-2 Microglobulin (β2-M); CCL11/Eotaxin; CD40; Ferritin H+L chain; Fibrinogen α/β/γ chain; Prostate specific antigen, free (PSA); Tissue inhibitor of metalloproteinase 1 (TIMP-1); Vascular cell adhesion molecule 1 (VCAM-1).

Additionally, the Elastic net model found 40 signaling proteins that significantly changed with age. These 40 predictors were used to build a classifier (f(x)=a₁x+a₂x+ . . . +a₄₀x) to calculate the age of the sample donors and compared on a scatter plot with the ideal linear function f(x)=x. A reasonable overlap between the calculated age with the actual calendar age of the donors was obtained, particular for donors 45 years and older.

TABLE 1
90 markers detected in human plasma.

| | | 44 markers different | 10 most robust |
|---|---|---|---|
| | | between age groups | predictors for |
| | | 20-45 y and ≧75 y | modeling age in |
| | Human Uniprot/ | (SAM analysis for q- | several scenarios |
| Protein name | SWISSPROT | value 0%)^{b} | (enet analysis) |
| α-1 Antitrypsin | P01009 | | |
| α-2 Macroglobulin | P01023 | − | |
| α-Fetoprotein | P02771 | | |
| Adiponectin/Acrp30 | Q15848 | | X |
| Apolipoprotein CIII (ApoC3) | P02656 | | |
| Apoliporotein H (ApoH) | P02749 | + | |
| Apolipoprotein A-1 (ApoA1) | P02647 | | X |
| β-2 Microglobulin (β2-M) | P01884 | + | X |
| Basic fetal growth factor (bFGF) | P09038 | − | |
| Brain-derived neurotrophic factor | P23560 | | |
| (BDNF) | | | |
| Complement factor 3 (C3) | P01024 | + | |
| Cancer antigen 125 (CA125) | Q14596 | − | |
| Cancer antigen 19-9 (CA19-9)^{a} | n/a | | |
| Calcitonin | P01258 | − | |
| Carcinoembrionic antigen (CEA) | P06731 | − | |
| | P78448 | | |
| CCL11/Eotaxin | P51671 | + | X |
| CCL2/MCP-1 | P13500 | + | |
| CCL22/MDC | O00626 | + | |
| CCL3/MIP-1α | P10147 | | |
| CCL4/MIP-1β | P13236 | − | |
| CCL5/RANTES | P13501 | | |
| CD40 | P25942 | + | X |
| CD40L | P29965 | | |
| Creatine kinase-MB (CK-MB) | P06732 | | |
| | P12277 | | |
| Creactive protein (CRP) | P02741 | | |
| CXCLS/ENA-78 | P42830 | − | |
| CXCL8/IL-8 | P10145 | | |
| Epidermal growth factor (EGF) | P01133 | | |
| Endothelin-1 | P05305 | − | |
| Erythropoietin (Epo) | P01588 | − | |
| Extracellular newly identified RAGE- | P80511 | − | |
| binding protein (EN-RAGE) | | | |
| Fatty acid binding protein 3 (FABP3) | P05413 | + | |
| Ferritin H + L chain | P02792 | | X |
| | P02794 | | |
| Fibrinogen α/β/γ chain | P02671 | + | X |
| | P02675 | | |
| | P02679 | | |
| Factor VII (FVII) | P08709 | | |
| Granulocyte colony stimulating factor | P09919 | | |
| (G-CSF) | | | |
| Granulocyte/macrophage colony | P04141 | | |
| stimulating factor (GM-CSF) | | | |
| Growth hormone (GH1) | P01241 | − | |
| Glutathion S-transferase (GSTA1) | P08263 | | |
| Haptoglobin (HP) | P00738 | + | |
| Intercellular adhesion molecule 1 | P05362 | + | |
| (ICAM-1) | | | |
| IgA | P01876 | | |
| IgE | P01854 | − | |
| IgM | P01871 | − | |
| Interleukin 1α (IL-1α) | P01583 | − | |
| Interleukin 1β (IL-1β) | P01584 | − | |
| Interleukin 1 receptor antagonist (IL- | P18510 | | |
| 1ra) | | | |
| Interleukin 2 (IL-2) | P01585 | | |
| Interleukin 3 (IL-3) | P08700 | | |
| Interleukin 4 (IL-4) | P05112 | | |
| Interleukin 5 (IL-5) | P05113 | | |
| Interleukin 6 (IL-6) | P05231 | − | |
| Interleukin 7 (IL-7) | P13232 | | |
| Interleukin 10 (IL-10) | P22301 | | |
| Interleukin 12p40 (IL-12p40) | P29460 | | |
| Interleukin 12p70 (IL-12p70) | P29459 | | |
| Interleukin 13 (IL-13) | P35225 | | |
| Interleukin 15 (IL-15) | P40933 | | |
| Interleukin 16 (IL-16) | Q14005 | + | |
| Interleukin 18 (IL-18) | Q14116 | + | |
| Insulin | P01308 | | |
| Insulin-like growth factor 1 (IGF-I) | P05019 | − | |
| Leptin | P41159 | | |
| Lipoprotein A (LPA) | P08519 | − | |
| Monocyte colony stimulating factor | P09603 | + | |
| (M-CSF) | | | |
| Matrix metalloproteinase 2 (MMP-2) | P08253 | + | |
| Matrix metalloproteinase 3 (MMP-3) | P08254 | | |
| Matrix metalloproteinase 9 (MMP-9) | P14780 | − | |
| Myeloperoxidese (MPO) | P05164 | − | |
| Myoglobin | P02144 | + | |
| Plasminogen activator inhibitor 1 | P05121 | + | |
| (PAI-1) | | | |
| Prostatic acid phosphatase (PAP) | P15309 | | |
| Pregnancy associated plasma protein | Q13219 | | |
| (PAPP-A) | | | |
| Prostate specific antigen, free (PSA) | P15309 | | X |
| Stem cell factor (SCF) | P21583 | | |
| Serum Amyloid P (SAP) | P02743 | | |
| Serum glutamic oxaloacetic | P17174 | | |
| transaminase (sGOT) | | | |
| Sex hormone-binding globulin | P04278 | + | |
| (SHBG) | | | |
| Thyroid stimulating hormone, α/β- | P01215 | | |
| subunit (TSH) | P01222 | | |
| Thyroxine binding globulin (TBG) | P05543 | | |
| Tissue factor (TF) | P13726 | | |
| Tissue inhibitor of metalloproteinase | P01033 | + | X |
| 1 (TIMP-1) | | | |
| Thrombopoietin (Tpo) | P40225 | | |
| Tumor necrosis factor-α (TNF-α) | P01375 | − | |
| Tumor necrosis factor-β (TNF-β) | P01374 | | |
| Tumor necrosis factor receptor II | Q92956 | + | |
| (TNFR-2) | | | |
| Vascular cell adhesion molecule 1 | P19320 | + | X |
| (VCAM-1) | | | |
| Vascular endothelial growth factor | P15692 | − | |
| (VEGF) | | | |
| Von Willebrand factor (vWF) | P04275 | | |
| XCL1/Lymphotactin | P47992 | − | |

^{a}CA19-9 is a carbohydrate cancer antigen and not a protein.
^{b}+, up-regulated with age; −, down-regulated with age.

### Statistical Analysis

Most of the statistical analysis was done in R (R Development Core Team (2009), available at world wide web address r-project “dot” org. Values were standardized before imputation by subtracting the mean and dividing by the standard deviation. Measurements below lowest detectable protein concentration for the 89 soluble proteins were imputed conservatively with lowest available value of a protein. Values missing at random were imputed with 0 of available observations after standardization. Regression models for continuous endpoints were computed with elastic net (Zou and Hastie, 2005) using the add-on package elasticnet, which is available from the hypertext transfer protocol address at cran “dot” r-project “rot” org/web/packages/elasticnet/index.html. The penalty parameters for the penalized linear regression models were chosen via 10-fold cross validation minimizing prediction error. For fixed alpha, the regularization parameter lambda is varied from lambda_max, the smallest lambda such that all coefficients are estimated without constraint, down to a very small lambda_min; lambda=0 renders the algorithm unstable (Friedman et al., 2008). We use the built-in specification of lambdas that automatically chooses a suitable number of lambdas in the interval [lambda_min, lambda_max]. For alpha we used {0.01, 0.2, 0.4, 0.6, 0.8, 1}. The alpha sequence was started at 0.01 because alpha=0 corresponds to a ridge regression penalty which does not produce reliable results (Friedman et al., 2008). Alpha=1 specifies a pure Lasso penalty. For the calculation of a predicted age in humans based on markers identified by enet, a classifier was built which is a linear regression function based on the actual measured value multiplied by the correlation coefficient for each selected marker. Differences between the two age groups 20-45 (n=49) and >75y (n=45) was analyzed by Significant Analysis of Microarray as two class Wilcoxon test.

Data are expressed as mean±SEM. Statistical analysis was performed with Prism 5.0 software (GraphPad Software). Means between two groups were compared with two-tailed, unpaired Student's t test. Comparisons of means from multiple groups with each other or against 1 control group were analyzed with 1-way ANOVA and Tukey-Kramer's or Dunnett's post hoc tests respectively. Plasma protein correlations in the aging samples were analyzed with the Significance Analysis of Microarray software (SAM 3.00 algorithm; available from the world wide web address at stat “dot” Stanford “dot” edu/˜tibs/SAM/index.htm). Unsupervised cluster analysis was performed using Gene Cluster 3.0 software and node maps were produced using Java Tree View 1.0.13 software.

Proteomic Screening of Biomarkers in Mouse Plasma and the Use of these Biomarkers to Assess the Age of Mouse

Mouse lines: C57BL/6 (The Jackson Laboratory, Bar harbor, ME), C57BL/6 aged mice (National Institutes of Aging, Bethesda, Md.), Dcx-Luc²¹, and C57BL/6J-Act-GFP (The Jackson Laboratory). All animal use was in accordance with institutional guidelines approved by the VA Palo Alto Committee on Animal Research. Mice were terminally anesthetized with i.p. injection of 0.4-0.7 mL 3.8% w/v chloral hydrate. 0.5-1.0 mL EDTA blood was taken by cardiac puncture and blood was kept on ice until further processing. Latest 2 hours after blood draw plasma was isolated by spinning the vacutainer at 1000 g for 10 minutes at 4° C. and followed by immediate aliquoting of the plasma and freezing at −80° C. Aliquots were not thawed until assayed. Detections of plasma concentrations of biomarkers were performed at Rules Based Medicine (Austin, Tex.) with an established and proprietary antibody-based multiplexed Luminex assay.

In a proteomic screening of plasma of healthy aging mice parallel to the proteomic approach of healthy aging human, we measured the concentrations of 53 secreted intercellular signaling proteins in 67 archived plasma samples from healthy aging mice (20 female and 20 male at 6, 12, 18, and 24 months of age) with flow cytometry-based Luminex technology and ELISA. 50 markers among these 53 signaling proteins were sufficiently detectable in mice plasma samples, as listed in Table 2.

Changes in these signaling molecules in the plasma of aging mice were measured to determine whether the molecular changes capable of modeling aging in humans can be adequately translated across species. Detailed procedures of plasma assaying and statistical methods were the same as those for human experiments described above. Measured results were compared between human and mouse samples and 12 significantly changing proteins were identified as conserved between species. It is noteworthy to mention that the twelve conserved markers do not necessarily represent the top predictors (define it) in mice. These identified 12 protein markers related to age in humans were used for unsupervised hierarchical clustering of the mouse samples, and were sufficient to produce a clear separation of mouse samples by age. Hence the identified pattern changes of the signaling proteins in plasma are capable of modeling biological processes such as healthy aging across species.

TABLE 2
50 markers detected in mouse plasma

| | | | 3 markers | 12 markers in mice |
|---|---|---|---|---|
| | | 4-6 markers | shared | that were shared in |
| | Mouse | that model | between | human to monitor |
| | NCBI Acc. | age well (enet | human and | age in several |
| Protein name | No. | analysis) | mice | different scenarios |
| Apolipoprotein A-1 (ApoA1) | NP_033822 | | | X |
| β-2 Microglobulin (β2-M) | NP_033865 | | X | X |
| Calbindin | NP_033919 | | | |
| CCL2/MCP-1 | NP_035463 | (X) | X | X |
| CCL3/MIP-1α | NP_035467 | | | |
| CCL5/RANTES | XP_122227 | | | |
| CCL7/MCP-3 | NP_038682 | | | |
| CCL9/10/MIP-1γ | NP_035468 | | | |
| CCL11/Eotaxin | NP_035460 | X | X | X |
| CCL12/MCP-5 | NP_035461 | | | |
| CCL19/MIP-3β | NP_036018 | | | |
| CCL22/MDC | NP_033163 | | | |
| CD40 | AAB08705 | | | X |
| CD40L | CAA46448 | | | |
| Clusterin | NP_038520 | | | |
| C reactive protein (CRP) | CAA31928 | | | |
| CXCL1,2,3/GRO-α,β,γ | NP_033166 | | | |
| CXCL6/GCP-2 | NP_033167 | | | |
| CXCL10/IP-10 | NP_067249 | X | | |
| Cystatin-C | NP_034106 | | | |
| Endothelial growth factor (EGF) | NP_034243 | | | |
| Endothelin-1 | NP_034234 | | | |
| Factor VII (FVII) | NP_034302 | | | |
| Growth hormone (GH1) | NP_032143 | | | X |
| Glutathion S-transferase (GSTa1) | NP_032207 | | | |
| Haptoglobin (HP) | NP_059066 | | | X |
| IgA | AAA38129 | | | |
| Interleukin 1α (IL-1α) | NP_034684 | | | |
| Interleukin 1β (IL-1β) | NP_032387 | | | |
| Interleukin 5 (IL-5) | NP_034688 | | | |
| Interleukin 6 (IL-6) | NP_112445 | | | |
| Interleukin 10 (IL-10) | NP_034678 | (X) | | |
| Interleukin 18 (IL-18) | NP_032386 | | | X |
| Insulin | NP_032412 | | | |
| Leptin | NP_032519 | | | |
| Leukemia inhibitory factor (LIF) | NP_032527 | | | |
| Lipocalin-2 | NP_032517 | | | |
| Monocyte colony stimulating | NP_031804 | | | X |
| factor (M-CSF) | | | | |
| Matrix metalloproteinase 9 | NP_038627 | | | |
| (MMP-9) | | | | |
| Myoglobin | NP_038621 | | | X |
| Osteopontin | NP_033289 | | | |
| Serum Amyloid P (SAP) | NP_035448 | | | |
| Serum glutamic oxaloacetic | NP_057911 | X | | |
| transaminase (sGOT) | | | | |
| Tissue factor (TF) | NP_034301 | | | |
| Tissue inhibitor of | NP_035723 | | | X |
| metalloproteinase 1 (TIMP-1) | | | | |
| Thrombopoietin (Tpo) | NP_033443 | | | |
| Vascular cell adhesion molecule 1 | NP_035823 | | | X |
| (VCAM-1) | | | | |
| Vascular endothelial growth factor | XP_192823 | | | |
| (VEGF) | | | | |
| Von Willebrand factor (vWF) | NP_035838 | X | | |
| XCL1/Lymphotactin | NP_032536 | | | |

Proteomic Screening of Biomarkers in Human Cerebrospinal Fluid and the Association of these Biomarkers to Age-Related Brain Disorders

In an attempt to more closely relate the systemic aging pattern discovered in plasma to the brain we started to analyze cerebrospinal fluid (CSF) samples from humans with AD (n=30) or healthy controls (n=31). CSF was isolated with in vitam lumbar puncture and without visible contamination with blood (Zhang et al., Am J Clin Pathol 2008) and aliquots were stored at −80° C. until analysis.

We measured again 89 proteins using Luminex-based assays. Unexpectedly, we were able to detect 58 out of the 89 proteins measured in at least 80% of the samples. Most of these factors have not previously been detected in CSF indicating that a much wider range of secreted signaling proteins is produced within the CNS. Moreover, 21 out of the 58 detectable proteins showed correlation coefficients R>0.4 or <*0.4 and 14 were detected at significantly different levels in AD versus controls. Collectively, these findings support a relationship between plasma and CSF and provide a basis for using plasma biomarkers of aging in the study of age-related brain disorders.

TABLE 3
73 markers detected in human CSF

| | Human Uniprot/ |
|---|---|
| Protein name | SWISSPROT |
| α-1 Antitrypsin | P01009 |
| α-2 Macroglobulin | P01023 |
| α-Fetoprotein | P02771 |
| Adiponectin/Acrp30 | Q15848 |
| Apolipoprotein CIII (ApoC3) | P02656 |
| Apoliporotein H (ApoH) | P02749 |
| Apolipoprotein A-1 (ApoA1) | P02647 |
| β-2 Microglobulin (β2-M) | P01884 |
| Basic fetal growth factor (bFGF) | P09038 |
| Complement factor 3 (C3) | P01024 |
| Cancer antigen 19-9 (CA19-9)^{a} | n/a |
| Calcitonin | P01258 |
| CCL2/MCP-1 | P13500 |
| CCL3/MIP-1α | P10147 |
| CCL4/MIP-1β | P13236 |
| CCL5/RANTES | P13501 |
| CCL11/Eotaxin | P51671 |
| CD40 | P25942 |
| CD40L | P29965 |
| Creatine kinase-MB (CK-MB) | P06732 |
| | P12277 |
| Creactive protein (CRP) | P02741 |
| CXCL5/ENA-78 | P42830 |
| CXCL8/IL-8 | P10145 |
| Endothelial growth factor (EGF) | P01133 |
| Endothelin-1 | P05305 |
| Erythropoietin (Epo) | P01588 |
| Extracellular newly identified | P80511 |
| RAGE-binding protein (EN-RAGE) | |
| Fatty acid binding protein 3 (FABP3) | P05413 |
| Ferritin H + L chain | P02792 |
| | P02794 |
| Fibrinogen α/β/γ chain | P02671 |
| | P02675 |
| | P02679 |
| Factor VII (FVII) | P08709 |
| Growth hormone (GH1) | P01241 |
| Glutathion S-transferase (GSTA1) | P08263 |
| Haptoglobin (HP) | P00738 |
| Intercellular adhesion molecule 1 (ICAM-1) | P05362 |
| IgA | P01876 |
| IgM | P01871 |
| Interleukin 1β (IL-1β) | P01584 |
| Interleukin 1 receptor antagonist (IL-1ra) | P18510 |
| Interleukin 4 (IL-4) | P05112 |
| Interleukin 5 (IL-5) | P05113 |
| Interleukin 6 (IL-6) | P05231 |
| Interleukin 7 (IL-7) | P13232 |
| Interleukin 10 (IL-10) | P22301 |
| Interleukin 12p70 (IL-12p70) | P29459 |
| Interleukin 13 (IL-13) | P35225 |
| Interleukin 15 (IL-15) | P40933 |
| Interleukin 16 (IL-16) | Q14005 |
| Interleukin 18 (IL-18) | Q14116 |
| Leptin | P41159 |
| Matrix metalloproteinase 2 (MMP-2) | P08253 |
| Matrix metalloproteinase 3 (MMP-3) | P08254 |
| Myeloperoxidase (MPO) | P05164 |
| Myoglobin | P02144 |
| Plasminogen activator inhibitor 1 (PAI-1) | P05121 |
| Prostatic acid phosphatase (PAP) | P15309 |
| Pregnancy associated plasma protein (PAPP-A) | Q13219 |
| Prostate specific antigen, free (PSA) | P15309 |
| Stem cell factor (SCF) | P21583 |
| Serum Amyloid P (SAP) | P02743 |
| Serum glutamic oxaloacetic transaminase (sGOT) | P17174 |
| Sex hormone-binding globulin (SHBG) | P04278 |
| Thyroid stimulating hormone, | P01215 |
| α/β-subunit (TSH) | P01222 |
| Thyroxine binding globulin (TBG) | P05543 |
| Tissue factor (TF) | P13726 |
| Tissue inhibitor of | P01033 |
| metalloproteinase 1 (TIMP-1) | |
| Thrombopoietin (Tpo) | P40225 |
| Tumor necrosis factor-α (TNF-α) | P01375 |
| Tumor necrosis factor-β (TNF-β) | P01374 |
| Tumor necrosis factor receptor II (TNFR-2) | Q92956 |
| Vascular cell adhesion molecule 1 (VCAM-1) | P19320 |
| Vascular endothelial growth factor (VEGF) | P15692 |
| Von Willebrand factor (vWF) | P04275 |

^{a}CA19-9 is a carbohydrate cancer antigen and not a protein.

### Example 2

### Age-Associated Changes in the Systemic Milieu Regulate Adult Neorogenesis

Immunohistochemistry was performed on free-floating sections following standard published techniques²⁸. Primary antibodies were against Dcx (1:500; Santa Cruz), BrdU (1:5000, Accurate Chemical and Scientific Corp.), Sox2 (1:200; Santa Cruz), GFAP (1:1500, DAKO), CD68 (1:50, Serotec), and β-dystroglycan (1:500, Novocastra Labs). Parabiosis surgery followed previously described procedures with the addition of surgical connection of the peritoneum¹⁷. Flow cytometric analysis was done on fixed and permeabilized blood plasma cells from GFP and non-GFP parabiotic pairings. Mouse neural progenitor cells were isolated from C57BL/6. NTERA cells and NPCs were cultured under standard conditions^{29,30}. Carrier free forms of recombinant Eotaxin/CCL11 (100 ng/ml) and β2-microglobulin (100 ng/ml) were added to cell cultures under self-renewal and differentiation conditions every other day following cell plating. Bioluminescence was detected with the In Vivo Imaging System (IVIS; Caliper) and quantified as photons/s/cm²/steridan (sr) using LIVINGIMAGE software (version 3.5, Caliper).

Decreased Adult Neurogenesis in the Dentate Gyrus During Aging Correlates with Changes in Secreted Plasma Proteins

Provided here are proteomic approaches to identify and use biomarkers to characterize age-related changes in nervous system such as reduced neurogenesis. For example, biomarkers identified by the proteomic analysis described herein are systemic biomarkers indicating the age-dependent decline in neurogenesis.

In this example, adult neurogenic niche in the aging mouse cohorts were characterized by assessing cellular changes in the dentate gyrus of the hippocampus at 6, 12, 18 and 24 months of age. Proteomic approach was employed in which the relative levels of 66 cytokines and secreted signaling factors were measured in the plasma of aging mice using antibody-based immunoassays on microbeads (Luminex; Table 4). Immunohistochemical analysis of Doublecortin (Dcx), a marker for newly differentiated neurons, indicated a greater than two-fold decline in the relative number of newly differentiated neurons between 6 and 12 months with a near complete absence of neurogenesis by 24 months. A similar decrease was also observed in the number of slow dividing BrdU-positive progenitors. Additionally, the multivariant analysis software Significance Analysis of Microarray (SAM) was used to search for significant changes in plasma signaling molecules that correlated with the decline in neurogenesis observed with age[88]. Seventeen biomarkers were identified using this analysis, exhibiting a false discovery rate (FDR) of less than 7%. Cluster analysis of the top correlated proteins produced a clear separation of samples according to the ages at which neurogenesis declines. Interestingly, factors identified through the proteomic analysis—such as Leptin, an adipose derived hormone—have been recently described as having an effect on neurogenesis in the adult hippocampus[89]. Another factor, TIMP-1, was also previously implicated in neurogenesis or NPC proliferation^{11,19,20}. Such results further validate this proteomic approach as a means to identify signaling protein markers directly involved in the regulation of nervous system processes such as neurogenesis.

TABLE 4
66 cytokines and secreted signaling factors measured in mouse plasma

| | Swiss-Prot |
|---|---|
| Protein Name | Accession Number |
| Apolipoprotein A1 | Q00623 |
| Beta-2-Microglobulin | P61769 |
| Calbindin | P12658 |
| CD40 | P27512 |
| CD40 Ligand | P27548 |
| Clusterin | Q06890 |
| C Reactive Protein | P14847 |
| Cystatin-C | P01035 |
| Epidermal Growth Factor | P07522 |
| Endothelin-1 | P22387 |
| Eotaxin | P48298 |
| Factor VII | P70375 |
| Fibroblast Growth Factor-9 | P54130 |
| Fibroblast Growth Factor-basic | Q9CWU6 |
| Fibrinogen | Q8KOE8 |
| Granulocyte Chemotactic Protein-2 | P80221 |
| Granulocyte Macrophage-Colony Stimulating | P01587 |
| Growth Hormone | P19795 |
| GST-alpha | P13745 |
| Haptoglobin | Q61646 |
| Interferon-gamma | P01580 |
| Immunoglobulin A | P01878 |
| Interleukin-10 | P18893 |
| Interleukin-11 | P47873 |
| Interleukin-12p70 | P43431 |
| Interleukin-17 | Q62386 |
| Interleukin-18 | Q14116 |
| Interleukin-1alpha | P01582 |
| Interleukin-1beta | P10749 |
| Interleukin-2 | P04351 |
| Interleukin-3 | P01586 |
| Interleukin-4 | P07750 |
| Interleukin-5 | P04401 |
| Interleukin-6 | P08505 |
| Interleukin-7 | P10168 |
| Insulin | P01325 |
| Inducible Protein-10 | P17515 |
| KC/GROalpha | P12850 |
| Leptin | P41160 |
| Leukemia Inhibitory Factor | P09056 |
| Lymphotactin | P47993 |
| Monocyte Chemoattractant Protein-1 | P10148 |
| Monocyte Chemoattractant Protein-3 | Q03366 |
| Monocyte Chemoattractant Protein-5 | Q62401 |
| Macrophage-Colony Stimulating Factor | P07141 |
| Macrophage-Derived Chemokine | Q54656 |
| Macrophage Inflammatory Protein-1alpha | P10855 |
| Macrophage Inflammatory Protein-1beta | P14097 |
| Macrophage Inflammatory Protein-1gamma | P51670 |
| Macrophage Inflammatory Protein-2 | P10889 |
| Macrophage Inflammatory Protein-3beta | 2404 (NCBI ID) |
| Matrix Metalloproteinase-9 | P41245 |
| Myoglobin | P04247 |
| NGAL | P11672 |
| Oncostatin M | P08721 |
| Osteopontin | P10923 |
| RANTES | P30882 |
| Stem Cell Factor | P20826 |
| Serum Glutamic-Oxaloacetic Transaminase | P05201 |
| Tissue Inhibitor of Metalloproteinase Type-1 | P12032 |
| Tissue Factor | P20352 |
| Tumor Necrosis Factor-alpha | P06804 |
| Thrombopoietin | P40226 |
| Vascular Cell Adhesion Molecule-1 | P29533 |
| Vascular Endothelial Cell Growth Factor | Q00731 |
| von Willebrand Factor | Q8C1Z8 |

Moreover, the findings of patterns of age-associated biomarkers are not only consistent with a decrease in adult neurogenesis¹⁶, additional findings also consistent with a concomitant increase in neuroinflammation with age. For example, an age-related increase of relative immunoreactivity to CD68, a marker for microglia activation and phagocytosis, was observed to increase, while the reactivity of GFAP-positive astrocytes did not change with age. Additionally, an age-dependent increase in β-dystroglycan-positive blood vessel staining was also observed between 12 and 18 months.

Heterochronic Parabiosis Reduces Adult Neurogenesis in Young Animals while Enhancing Neurogenesis in Aged Animals

Parabiotic pairings between young and old mice were established to determine if age-related cellular changes in the hippocampus are encoded by intrinsic factors within the local environment in the CNS or may be the result of changes in the peripheral milieu. To examine the influence of the aging systemic milieu on adult neurogenesis, vasculature between young (3-4 months) and aged (18-20 months) mice was connected using isochronic (young-young and old-old) and heterochronic (young-old) parabiotic pairings. Briefly, mice were anesthetized to full muscle relaxation with isofluorane (1-4%, to effect) inhalation. On the first adjoining mouse a skin incision extending in a curve from the side of the elbow to the knee was made. Skin was freed from the subcutaneous fascia allowing the elbow and knee joints to be accessible. A second incision was made to the peritoneum. Mirror image incisions were then made in the adjoining mouse as described above. The peritoneum from both animals was adjoined using absorbable sutures. Knee and elbow joints were then sutured together as to optimize coordinated locomotion. Skin on the dorsal and ventral sides was stapled together using 9 mm Autoclip. Skin around the joints that was inaccessible to autoclips was sutured together. A joined systemic environment was confirmed by flow cytometry in 4 sets of paired mice in which one parabiont of each pair was transgenic for green fluorescent protein (GFP) under the control of the actin promoter and the other parabiont was wildtype. Approximately 40-60% of cells in the blood of either parabiont were GFP-positive after 2 weeks of parabiosis (FIG. 5). In contrast, no GFP-positive cells were detected in brains of wildtype parabionts, which confirms that blood cells do not normally enter the brain in the absence of injury [90]. By immunocytochemical analysis, Dcx-positive neurons in young heterochronic parabionts was observed to decrease 20% compared to young isochronic parabionts. Likewise, BrdU-positive cells and Sox2-positive progenitors showed a similar decrease. Interestingly, there was a 3-fold increase in the number of Dcx-positive neurons, and BrdU-positive cells in the aged heterochronic parabionts compared to isochronic parabionts. The number of Dcx-positive neurons between unpaired age-matched animals and old isochronic animals showed no significant difference.

The dendritic length of newly differentiated neurons in normal and heterochronic parabionts were also compared. There was a natural decline in dendritic length between 12 and 18 months of age. Young heterochronic parabionts showed a 20% decrease in length compared to isochronic parabionts. Conversely, 18-month heterochronic parabionts demonstrated a 40% increase in length, similar to that observed in unpaired 12-month old mice, when compared to age-matched isochronic controls. These results indicate that the peripheral milieu can promote morphological changes. In summary, global age-dependent molecular changes in the systemic milieu can modulate neurogenesis in both the young and aged neurogenic niche, contributing to the decline in regenerative capacity observed in the aging brain. Since no cells appear to enter the CNS, these effects are most likely mediated by soluble factors in plasma.

Changes in Concentration of Certain Secreted Plasma Proteins Correlate with Declining Neurogenesis Observed During Both Aging and Heterochronic Parabiosis

Plasma samples from young and aged animals before and 5 weeks after pairings were analyzed to examine molecular changes associated with parabiosis. Comparison of young isochronic and heterochronic cohorts identified fourteen factors with a greater than 2-fold increase in expression in the heterochronic parabionts (Table 5). Conversely, a comparison between old isochronic and heterochronic cohorts revealed four factors whose expression levels decrease to less than 70% of that observed in isochronic parabionts (Table 5). Interestingly, five factors—Eotaxin/CCL11, β2-microglobulin, MCP-1, MCP-5 and Haptoglobin—were elevated in both aged unpaired and young heterochronic cohorts compared to young unpaired or isochronic cohorts. These factors were then evaluated and used as a correlate of an aging systemic environment. Changes in plasma concentrations of these factors occurring within individual animals during normal aging were compared to those occurring within young individual animals during heterochronic parabiosis, and an increase of Eotaxin/CCL11, β2-microglobulin, and MCP-1 were observed. Hence, these factors both recapitulate an aged systemic environment and correlate with decreased neurogenesis, and thus may serve to pinpoint systemic factors that influence the decline in neurogenesis observed during aging and heterochronic parabiosis.

TABLE 5
Molecular changes between isochronic and heterochronic
parabiotic groups.

Fold Change
(versus Isochronic)

| | Young | Old |
|---|---|---|
| Protein Factor | Heterochronic | Heterochronic |
| β2-Microglobulin | 17.7 ± 1.7 | n.c |
| Haptoglobin | 8.5 ± 0.6 | −1.4 ± 0.1 |
| IL-11 | 8.5 ± 1.4 | 4.6 ± 1.5 |
| KC/GROα | 7.3 ± 1.1 | n.c |
| IL-1α | 6.5 ± 1.1 | −1.4 ± 0.1 |
| IL-7 | 6.5 ± 1.0 | n.c |
| GCP-2 | 3.5 ± 0.4 | n.c |
| MIP-1β | 2.9 ± 0.3 | n.c |
| Myoglobin | 2.9 ± 0.6 | n.c |
| MPO | 2.8 ± 0.3 | n.c |
| MCP-1 | 2.3 ± 0.2 | n.c |
| MIP-3β | 2.2 ± 0.1 | n.c |
| IL-5 | 2.2 ± 0.2 | −1.6 ± 0.04 |
| Eotaxin/CCL11 | 2.1 ± 0.3 | n.c |
| MCP-5 | 2.1 ± 0.1 | n.c |
| CD40 | n.c | −1.4 ± 0.1 |

Note:
Signs of fold change indicate the change of expression level of factors in plasma: positive signs indicate increases of the factors in plasma concentrations and negative signs indicate decreases of the factors in plasma concentrations (mean ± SEM of fold changes observed with parabiosis; n.c. denotes no detectable change).

These factors were further evaluated to corroborate molecular changes correlating with decreased neurogenesis in mice to those changes occurring in humans. Eotaxin/CCL11, β2-microglobulin and MCP-1 in archived plasma and cerebral spinal fluid (CSF) samples were measured from healthy individuals between 20 and 90 years old. Indeed, an age-related increase in Eotaxin/CCL11, β2-microglobulin and MCP-1 measured in both plasma and CSF were detected, suggesting that these systemic age-related molecular changes are conserved across species. Because the observed molecular changes correlate with decreased adult neurogenesis it may represent a common biological source contributing towards diminishing tissue regeneration in the aging brain.

### Biological Relevance of Individual Biomarkers in NPC Function

Provided herein are also the analysis of biological relevance of the biomarkers on NPC functions, such as NPC proliferation and neurogenesis, in cell culture models and in vivo. These biomarkers are identified herein to be associated with aging systemic environment and correlated with decreased neurogenesis.

Primary mouse NPC cultures were used to evaluate the effect of biomarkers on NPC functions. After four-day exposure to recombinant β2M or Eotaxin, the number and diameter of neurospheres formed were observed to decrease compared with control conditions. Neurogenesis was assayed using a human derived NTERA cell line expressing eGFP under the Doublecortin promoter. Decreased eGFP expression was detected after twelve days in culture with β2M or Eotaxin under differentiation conditions with retinoic acid.

Evaluation of the effect of biomarkers on NPC functions was performed in vivo. In one example, stereotaxic injection of recombinant β2M was performed to attempt to inhibit neurogenesis in vivo. Specifically, recombinant β2M were injected stereotaxically into the right dorsal hippocampus (coordinates from bregma: A=−2.0 mm and L=−1.8 mm, from brain surface: H=−2.0 mm) under Isofluorane anesthesia. Recombinant β2M was injected over 2 min using a 5-μl Hamilton syringe. After injection, the needle was maintained in situ for an additional 2 min to limit reflux along the injection track. The skin was closed using adhesive surgicalblock and each mouse was injected subcutaneously with Buprenex as directed for pain relief. Seven days following surgical procedure animals were sacrificed and tissue processed for neuropathological analysis. Using immunocytochemistry, a decrease in the number of Dcx-positive neurons in the dentate gyrus, injected with recombinant β2M, was observed compared to the vehicle control side. To complement overexpression experiments, the relative number of proliferating NPCs in a pilot study was also analyzed with a small cohort of β2M knock-out mice lacking expression of endogenous β2M. The adult neurogenic niche has been shown to contain both slow dividing quiescent stem cells and highly proliferative progenitors are present. To characterize total changes in the number of NPCs, proliferation of both slow and rapidly dividing cells was assessed by detecting incorporation of BrdU and/or Ethynyldeoxyuridine (EdU), as both thymidine analogues incorporate in DNA during the S phase of cell division. Specifically, one week of BrdU (5 mg/ml) injections were followed two weeks later by one week of EdU (5 mg/ml) injections, thus labeling two distinct populations based on proliferation rates Animals were then sacrificed one day following the last injection and tissue processed. A decrease in the number of dividing BrdU and EdU-positive cells was observed, suggesting that the absence of β2M can promote NPC proliferation.

As another example, Eotaxin/CCL11 was elevated to attempt to inhibit neurogenesis in vivo. Eotaxin/CCL11 is a factor identified to increase in the aged systemic environment. Changes in neurogenesis within the same mouse were monitored with a non-invasive bioluminescent imaging assay using Doublecortin-luciferase reporter mice²¹. The relative change in the number of Doublecortin-positive cells was determined by changes in luciferase activity. Carrier-free recombinant murine Eotaxin/CCL11 protein was administered through intraperitoneal injections into 3-4-month-old mice every other day for 4 days Animals were imaged on day 0 and 4. A significant decrease in luciferase activity was detected in animals receiving recombinant Eotaxin/CCL11 compared to vehicle controls. Therefore, the over-expression of even a single age-related factor is sufficient to partially recapitulate the inhibitory effect of aging or heterochronic parabiosis on neurogenesis.

In summary, age-related molecular changes occurring in the systemic milieu can diminish adult neurogenesis. While it is expected that peripheral factors capable of modulating neurogenesis may be precluded by the blood-brain barrier (BBB), three-dimensional imaging of the vascular interactions with NPCs have recently revealed the absence of a classical BBB²²⁻²⁴, potentially allowing blood-derived factors access to the neurogenic niche. Therefore changes in systemic factors during aging could manifest functionally in the brain as diminished neurogenesis.

Previous studies investigating molecular changes during global aging and neurodegeneration have focused on transcriptional targets. While transcriptomes are in the order of tens of thousands of genes, it is estimated that 800-1000 secreted signaling proteins in plasma comprise the bulk of intercellular communication factors and thus a key part of the systemic milieu. These factors termed the communicome²⁵ may provide a more targeted platform for investigating age-related molecular changes and their functional role in the aging brain. 66 cytokines were assayed, which are less than 10% of the total signaling molecules present in plasma, and biomarkers such as Eotaxin/CCL11 and β2-microglobulin were identified as biologically relevant inhibitory factors in the CNS.

While in the periphery Eotaxin/CCL11 and β2-microglobulin are classically involved in inflammatory immune responses, a functional role for Eotaxin/CCL11 in the CNS had not been identified, and while β2-microglobulin expressed in the developing cortex has been shown to be involved in synaptic plasticity²⁶, peripherally derived β2-microglobulin has not been studied. The results from the present invention, however, suggest a communication between the periphery and the aging CNS. Molecular changes in the systemic milieu of human patients can correlate with and are capable of predicting susceptibility to neurodegenerative diseases such as Alzheimer's and Huntington's disease^{25,27}. These systemic changes may influence the onset and progression of age-related neurodegenerative diseases, hence providing a novel avenue to explore therapeutic targets.

### Example 3

### Modulation of NPC Proliferation and Differentiation In Vitro by β2M

Without being bound by theory, we suggest that β2M signaling results in decreased NPC proliferation, self-renewal and neuronal differentiation while abrogation of β2M enhances these functions.

Soluble β2M in the periphery has been shown to directly influence the biology of different cell types in a pleomorphic manner independent of its classical role in the adaptive immune system [67, 68]. In vitro studies using cancer cell lines have also indicated that such cell specific effects by β2M can occur through non-canonical signaling mechanisms independent from its association with MHC1 molecules [1, 2]. To date, work in the CNS has shown that intrinsic β2M functions in synaptic plasticity during both cortical development and in response to injury. β2M's role, however, has been attributed entirely to its involvement with MHC1 molecules [80, 81]. While β2M can act both in conjunction with and independent from MHC1 molecules, the direct influence of soluble β2M in the CNS has not been investigated. Embodiments of the present invention have identified β2M as an age-related systemic factor associated with decreased NPC function. We have demonstrated that β2M can act directly on NPCs in vitro by inhibiting proliferation and self-renewal, as well as impede neuronal differentiation in a teratoma derived cell line. The embodiments presented here determine whether β2M signaling is both necessary and sufficient for NPC function, determine the signaling mechanism by which β2M acts, and identify potentially novel non-canonical receptors for β2M expressed by NPCs.

### Sufficiency and Necessity of β2M in NPC Function

Recombinant β2M administration in cell culture performed herein suggested that β2M is sufficient to inhibit NPC function. Its role in neuronal and glial differentiation, however, has not been investigated. Equally important the relative necessity of β2M for NPC proliferation, self-renewal or differentiation is unknown. Hence it is to be determined whether exogenous β2M can impede neuronal and/or glial differentiation, as well as, whether long-term or acute deletion of β2M can enhance NPC function.

Our in vitro findings on β2M through recombinant β2M administration in cell culture will be corroborated with an independent approach using an adenovirus-mediated overexpression model. Viral constructs that overexpress the human form of β2M have been generated using adenoviral vectors (Mayo Clinic, Jacksoville, Fla.) and the efficacy of expression in cells have been confirmed using a CHO cell line. The sufficiency of β2M to inhibit NPC proliferation and self-renewal will be confirmed using the NPC neurosphere assay as described in Example 2. To ensure a homogenous progenitor population all experiments should be done with cultured NPCs that have undergone at least four passages. Dissociated primary mouse NPCs will be infected under self-renewal conditions (addition of EGF and bFGF) and their ability to form neurospheres will be measured 4-6 days later. Over-expression of β2M will be confirmed using Western blot analysis. The self-renewal potential of the NPC will be assayed by quantifying the total number of primary and secondary derived neurospheres formed after viral infection. Proliferation will be assayed by quantifying the average diameter of neurospheres formed after infection. To complement these studies Bromodeoxyuridne (BrdU) will be added to dissociated neurospheres, and the total number of BrdU-positive cells per individual neurosphere will be measured as an additional reference for proliferation.

Next, effects of exogenous β2M on multipotency will be assessed by replating single neurospheres and adherently culturing them for 3-5 days under differentiation conditions (addition of retinoic acid in the absence of EGF and bFGF) after either recombinant β2M administration or viral infection. Immunoctyochemistry will be used to stain molecular markers for neurons (Tuj1, Map2), astrocytes (GFAP, 510013) and oligodendrocytes (olig2). The percentage of neurospheres capable of giving rise to all three cell types will be quantified as a reference for multipotency [41, 42, 92]. Neurogenesis and gliogenesis will be assayed using an in vitro bioluminescent approach. We have currently available bioluminescent reporter mice that express luciferase under the control of either the Dcx or GFAP promoters. Primary cortical postnatal NPCs will be isolated, as they do not express Dcx or large numbers of GFAP+ cells until differentiation is initiated [93]. Dissociated cells will either be exposed to recombinant β2M or infected with adenovirus overexpressing β2M. Following neurosphere formation growth factors will be withdrawn and retinoic acid added for 6-8 days to induce differentiation. Changes in the number of newly differentiated neurons or glia will be assayed by bioluminescence imaging and enzymatic activity.

We have demonstrated that exogenous β2M plays an inhibitory role in NPC function (FIG. 10). We thus suggest that the proposed viral experiments can result in a similar inhibition of NPC proliferation and self-renewal. Additionally, we have also demonstrated in vivo a decrease in neurogenesis after stereotaxic injection of β2M into the hippocampus. Likewise, we suggest both pharmacologically and virally presented β2M can inhibit differentiation. Collectively, these studies will demonstrate that β2M is sufficient to inhibit NPC function and differentiation at a cellular level.

The next of experiments will examine the necessity of β2M in NPC function under self-renewal and differentiation conditions using a combination of neurosphere and bioluminescent cell culture models. We have established a β2M knock-out colony to investigate the long-term effect of β2M in NPC function. Primary cortical postnatal NPCs will be isolated from knock-out animals, and proliferation, self-renewal, and multipotency will be assayed. Additionally, the effect of acute β2M-abrogation on NPC function will be examined using an adenoviral-based RNA interference (RNAi) approach. In this regard, adenoviruses encoding at least two independent shRNA sequences, which downregulate expression of β2M, will be generated and efficacy will be confirmed using Western blot analysis. We have substantial experience using overexpressing or shRNA encoding lenti and adenoviruses[94]. The relative changes in proliferation and differentiation will be assessed by comparing neurospheres, as well as bioluminescent reporter cells, derived from RNAi infected cells versus cells infected with a non-specific scrambled sequence. All proliferation and differentiation assays will follow the same immunocytochemical and luciferase strategies described above.

We have suggested the absence of endogenous β2M results in an increase in the number of slow-dividing stem cells, when looking at long-term and short-term BrdU retention in β2M knock-out mice. Therefore, we suggest that abrogating the expression of β2M will increase proliferation and self-renewal of NPCs, and may also result in an increase in differentiation. Collectively, these studies will demonstrate that endogenous β2M in NPCs is necessary to inhibit NPC function and differentiation at a cellular level.

The Molecular Signaling Mechanism by which β2M Functions in NPCs

Previous studies examining the effect of soluble β2M on cellular processes have demonstrated that β2M can directly regulate renal cancer cell proliferation and survival through activation of the MAPK/ERK signaling pathway[69]. In the CNS, ERK1/2 have been shown to be activated after phosphorylation of Threonine and Tyrosine residues in response to extracellular stimuli including neurotransmitters, neurotrophins [95], growth factors [96, 97], and some pathological conditions such as brain ischemia [98, 99]. Interestingly, activation of ERK1/2 results in an increase of NPC proliferation, neurogenesis[100, 101], synaptic plasticity and learning and memory in the adult hippocampus [102-104]. Given the interaction between soluble β2M and the MAPK/ERK pathway, and the involvement of ERK signaling in NPC function, one candidate downstream pathway of β2M's inhibitory effect on NPCs is ERK1/2. To explore this possibility we assessed activation of ERK1/2 in primary NPCs in response to β2M. Using Western blot analysis we detected a dose dependent decrease in phosphorylated ERK1/2 (pERK1/2) in NPCs cultured in the presence of recombinant β2M.

The inhibition of ERK1/2 by β2M and its effect on NPC function in vitro will be further examined biochemically. First the effect of β2M on pERK1/2 levels will be examined. Primary NPCs under self-renewal conditions will be cultured in presence of recombinant β2M (1 μg/ml) and temporal changes in the level of pERK1/2 at 0, 1, 3, 6, 12, 24 and 48 hours will be assessed by Western blot using a polyclonal antibody to phosphorylated ERK1/2 at threonine 202 and tyrosine 204 (Cell Signaling). In addition, the dose response curve will be repeated and expanded to about 0.1 μg/ml-50 μg/ml of recombinant β2M, which can be observed in plasma of patient with chronic kidney disease. We will complement pERK1/2 expression analysis with kinase activity assays. A nonradioactive p44/42 Kinase Assay kit will be used as previously reported (Cell Signaling)[105]. Cells will be plated in six-well plates, treated with recombinant β2M, and lysed. Immobilized active pERK1/2 will be immunoprecipitated on beads coupled to pERK1/2 antibody and collected from lysates after treatment with β2M. The bead-bound pERK1/2 will then be incubated with the kinase assay substrate ELK. Levels of phosphorylated ELK will be determined by immunoblot using an antibody to phosphorylated ELK (pELK). Relative expression of pELK will be used as a readout of pERK1/2 activity.

Next, the investigation will focus one whether activation of ERK1/2 signaling can rescue the inhibitory effect of β2M on NPC proliferation and/or differentiation. Dissociated NPCs isolated from postnatal Dcx-Luc mice will be transiently transfected with plasmids encoding for the conditional protein kinase ΔRaf-1:ER, which selectively activates ERK1/2 in response to treatment with 4-hydroxytamoxifen (4-HT)[106]. NPC proliferation and self-renewal after exposure to recombinant β2M in cells expressing the constitutively active form of ERK1/2 will be examined using the neurosphere assay. Differentiation will be assayed by bioluminescence imaging and enzymatic activity as described above. The results indicate that endogenous pERK1/2 decreases in response to β2M in primary NPCs, which suggests that the decrease in NPC proliferation and neuronal differential is in part attributable to the decrease in ERK1/2 signaling. Therefore, we expect that introducing a constitutively active form of ERK1/2 in the presence or β2M will rescue to the inhibitory effect exerted by β2M, resulting in increased neurosphere number, size and neuronal differentiation. If the ERK pathway does not show prominent effects, other signaling pathways reported to be regulated by β2M, including AKT/PI3K [69] and PKA [1, 2], may also be explored.

To obtain more unbiased information on how β2M may regulate signaling, transcription factors activated by β2M will be identified using the Transignal Transcription Reporter Array (Panomics) which allows for the measurement of transcriptional activity of up to 100 transcription factors. Primary NPCs or an adult rat NPC cell line can be transiently transfected with a provided reporter plasmid mix (containing 20-50 specific reporter plasmids). Cells will be treated with β2M or vehicle control. This will lead to the production of specific artificial RNA tags from activated reporter plasmids. Total RNA will be extracted after 4 hours, and biotin-labeled cDNA probes will be prepared and hybridized to an array with complementary tags. The blot will then be developed with streptavidin-HRP, and chemiluminescence signals will be measured to determine the relative abundance of each transcription factor (see for example Sussan et al. [107]).

### Identify Non-Canonical Receptors for β2M Present in NPCs

β2M has been traditionally thought to function as a component of the MHC1 molecules. However, studies investigating cell growth in prostate cancer cells lacking expression of MHC1 suggest that the effects of soluble β2M on cellular function are mediated in part via novel non-canonical receptors [1, 2]. To explore if non-canonical receptors involve in mediating β2M in NPCs we will use an immunoprecipitation approach in conjunction with mass spectrometry. Recombinant β2M will be biotinylated using a standard biotinylation kit (Sigma Aldrich, St. Louis, Mo.). Adult rat derived NPC cell line described above will be used as it can be readily amplified in a large scale, to obtain protein quantities necessary for the assays proposed below. NPCs with biotinylated β2M will be cultured for 24 hours and cells are homogenized. The cell membrane will be isolated by cellular fractionation using sequential centrifugation. Specifically, cell homogenate will be centrifuged at 7,500 rpm to pellet the nucleus. The supernatant containing the cytosol and membrane will then be centrifuged at 25,000 rpm to isolate the cell membrane as a pellet. Membrane bound proteins will be extracted using RIPA buffer. Proteins bound to β2M will be sequestered by immunoprecipitation of biotinylated β2M using affinity chromatography with beads coupled with streptavidin. Purification of β2M will be confirmed by Western blot. We will then screen for candidate receptors by examining purified β2M by tandem mass spectrometry (MS/MS). β2M can be crosslinked to its putative receptor using photoactivatable crosslinkers (Pierce, Rockford, Ill.) in combination with the above protocol or by immunoprecipitation with antibodies specific for β2M. If the receptor is a known protein, molecular and cell biology techniques can be used to express or delete the receptor in CHO or other cells to establish its function as a β2M binding protein.

Most of the methods proposed in this Example are routinely used without any significant technical challenges. For example, we have significant experience in the use of viral vectors in cell culture and mice ([94]. The “in suspension” nature of the mouse neurosphere assay may prevent the use of an adhesive substrate to propagate NPC passages, and thus may render quantifiable conclusions about NPC proliferation and multipotency more difficult than traditional adherent culture systems. Nevertheless, being critical of NPC isolation by confirming renewal of the founding population of cells over an extended period of time coincident with the generation of a large number of progeny should enable proper pharmacological and viral investigation of NPCs. Additionally, while the use of primary reporter cells will enable the assaying of total changes in differentiation, it may not allow the thoroughly assessing of relative changes in neurogenesis versus gliogenesis that may be occurring at the level of individual neurospheres. Hence these studies may be further complemented with immunocytochemical investigations in which the percentage of cells that express Dcx or GFAP per neurosphere can be quantified.

### Example 4

### Increasing Peripheral β2M to Affect the Age-Dependent Decline in NPC Function In Vivo

Without being bound by theory, we suggest that increasing levels of peripherally derived β2M exacerbate the age-related decline in NPC proliferation and neurogenesis in the adult brain.

Recent studies characterizing the cellular composition of the adult neurogenic niche have demonstrated that the vascular interactions with adult NPCs are devoid of a classical BBB thus enabling blood-derived systemic factors, such as β2M, to access to the stem cell niche [24-27]. We have provided evidenced that age-related increases in plasma levels of β2M correlate with decreased adult neurogenesis during both normal aging and heterochronic parabiosis (Table 5). Additionally, in vivo stereotaxic injections of recombinant β2M into the dentate gyrus of the hippocampus in young adults result in a decrease in neurogenesis, suggesting that changes in β2M levels influence the decline in NPC function occurring during aging. In this Example, we will determine whether overexpression of either exogenous β2M in the CNS or periphery is capable of decreasing NPC function in the young and aged brain and we will also explore the associated cognitive impairments. The experiments proposed here will help elucidate the role of systemic β2M in the declining regenerative potential observed in the aging brain.

We have shown via stereotaxic injection delivery of recombinant β2M into the adult dentate gyrus that exogenous β2M directly delivered to the CNS results in a decrease in neurogenesis. Therefore, we conclude that both pharmacological and viral delivery of β2M into the brain can decrease proliferation and self-renewal of NPCs. Collectively, these studies provide evidence of the inhibitory effect of exogenous β2M in the CNS.

### Determine how Systemically Derived β2M Affects NPC Proliferation and Differentiation

Using a targeted proteomic screening, we have identified β2M as a key systemic factor whose expression levels increase in association with decreasing levels of adult neurogenesis during normal aging. Additionally, proteomic analysis of plasma taken from young adult mice prior to and following heterochronic parabiosis also identified β2M as a systemic factor whose levels increase in association with decreased neurogenesis (Table 5). Given the direct inhibitory role exogenous β2M has on NPC function in vitro and in vivo, we elucidated the specific role that systemically derived β2M has on decreased NPC function. To examine this, β2M was overexpressed in the periphery of both normal aging animals and heterochronic parabionts.

Proteomic data indicated that systemic changes in the order of 500 ng/ml occur in the plasma of aging mice between 6 and 24 months of age. Additionally, in vitro findings showed that administration of β2M at a concentration of 100 ng/ml is sufficient to inhibit NPC function. Therefore, 500 ng/ml of recombinant β2M will be administered through intraperitoneal injections into adult mice. β2M will be administered every other day for two weeks to ensure increased levels of β2M accumulate in the systemic milieu of animals.

We have shown that exposure of a young neurogenic niche to an aged systemic environment through heterochronic parabiosis can directly diminish NPC proliferation and neurogenesis in the young brain. Consistently, no changes were detected in the number of infiltrating cells from the periphery in parabionts, which indicated that the phenotypic changes observed are most likely mediated by systemic factors delivered by blood. Given the inhibitory effect of β2M in cell culture, as well as in vivo with stereotaxic injection delivery, we have shown that the decline observed in young heterochronic parabionts is mediated by increased levels of peripheral β2M.

TABLE 6
Combinations of heterochronic parabiosis pairings.

| | |
|---|---|
| ANIMAL | PARABIOTIC PAIR |

| AGE | 2 months | 18 months |
|---|---|---|
| GENOTYPE | β2M^{-/-} | WT; AAV-β2M |
| | WT; AAV-β2M | β2M^{-/-} |
| | β2M^{-/-} | WT; AAV-Control |
| | WT; AAV-Control | β2M^{-/-} |

β2M^{-/-}: denotes knock-out animals lacking β2M expression.
AAV-control: denotes animals infected with control adenovirus.
AAV-β2M: denotes animals infected with adenovirus encoding human β2M.

We have demonstrated through parabiosis that age-related changes in the levels of systemic factors inhibit neurogenesis in the adult brain, and furthermore identified β2M as one such factor. We therefore have shown that the reintroduction of systemic β2M from the adjoining wild-type parabiont overexpressing β2M to parabionts lacking endogenous β2M will result in decreased NPC proliferation and neurogenesis compared to parabiotic pairs in which no β2M is expressed in either animal. We have shown the specific inhibitory effect of systemically derived β2M in the adult neurogenic niche.

### Example 5

### Deletion of β2M to Mitigate the Age-Dependent Decline in NPC Function In Vivo

We have shown that abrogation of β2M can mitigate the age-related decline in NPC function and concomitantly influence rejuvenation in the aged brain.

Our parabiosis studies in the brain demonstrated that the rejuvenating ability persists within the aged neurogenic niche, i.e., exposure aged NPC to a young systemic environment results in increased regeneration. Results from young adult β2M knock-out mice suggested that the absence of β2M promotes the proliferation of dividing NPCs in vivo, thereby maintaining a larger pool of available stem cells. Because exogenous β2M inhibits NPC proliferation and differentiation, and because systemic levels of β2M increase in plasma and CSF in aging organisms, we suggest that decreasing levels of β2M mitigates the age-related decline in NPC function. In this example, NPC proliferation and differentiation will be characterized after deletion of β2M in young and aged animals, and individual effects associated with decreasing CNS-derived versus systemically derived β2M on NPC functions will be determined. Additionally, issue regarding whether lack of β2M can lead to an enhancement in cognitive function will be discussed. Accordingly, we have identified β2M as a novel therapeutic target accessible in the systemic environment providing an avenue by which to combat or prevent age-dependent degeneration and neurodegenerative diseases.

We have shows evidence for the use of the in vivo bioluminescence bioluminescence approach to detect changes in adult neurogenesis in consistent with age-related molecular changes identified in the proteomic screen described herein. Eotaxin has been identified as a factor secreted by microglia capable of inhibiting neurogenesis in vitro[115]. Consistently we have identified Eotaxin/CCL11 as an age-related systemic factor capable of decreasing NPC proliferation in vitro. As described in Example 2, through non-invasive bioluminescent imaging assay using Dcx-lufiferase reporter mice, systemically administered recombinant Eotaxin/CCL11 in promoted a significant decrease in luciferase activity compared to vehicle controls (FIG. 10F, 10G), indicating a inhibited neurogenesis. These findings also demonstrate the feasibility of in vivo imaging techniques, and their ability of providing evidences for the biological relevance of the proteomic approach described herein.

The results in young adult β2M knock-out mice suggested that the absence of endogenous β2M expression can increase the number of proliferating progenitors in the DG. These results can be corroborated in young adult mice lacking β2M expression. Additionally, we showed that the absence of endogenous β2M during the aging process can result in an amelioration of the age-related decline in NPC number and adult neurogenesis observed in the aged brain.

### Investigate Effects of Decreasing CNS Versus Systemically Derived β2M on NPC Function

Previous studies investigating the effect of extracellular growth factors in regulating neurogenesis have employed peripheral administration of neutralizing antibodies to abrogate the effect of such factors in vivo [1, 2, 116, 117]. For example, immunoneutralization of basic fibroblast growth factor (bFGF) resulted in a decrease in NPC proliferation compared to heat-inactivated control antibodies [118]. To directly examine the role of systemically derived β2M on NPC proliferation and differentiation during aging, we down regulated the expression of available endogenous β2M in the systemic milieu by peripheral administration of commercially available polyclonal neutralizing antibodies against β2M (Santa Cruz Biotechnology, Santa Cruz, Calif.) [2]. Neutralizing antibodies were injected intraperitoneally into young (2-4 months) and aged (16-18 months) mice (n=8 per group, equal sex distribution) every other day for 4 weeks to ensure inhibition of endogenous β2M. Using immunohistochemistry, the relative proliferation of adult NPCs in response to inhibition of systemic β2M was assessed by detecting both short-term Edu and long-term BrdU incorporation. NPC populations in vivo were also examined with mouse monoclonal antibodies to Nestin and Sox2. In order to investigate neuronal and glial differentiation we quantified the total number of BrdU-positive cells that double label with either Dcx and NeuN neuronal markers, the astrocyte marker GFAP or 510013, and the oligodendrocyte marker olig2 or NG2.

We have identified β2M as a key age-related factor associated with decreased NPC function and suggested that the increase in systemic levels of β2M observed in aging and heterochronic parabiosis directly influence the decrease in adult neurogenesis. Accordingly, we have shown that down regulation of β2M, particularly in the systemic environment, can result in an increase in adult NPCs and neurogenesis, thus mitigating the decline in regenerative capacity normally observed during aging. Further, differentially abrogating the expression of β2M can elucidate differences in the inhibitory effect of CNS versus systemically derived β2M in the adult neurogenic niche.

### Determine Whether Lack of β2M Results in Enhanced Learning and Memory

Cognitive functions, such as learning and memory abilities, can be evaluated with a classical Morris Water mazes protocols. β2M knock-out and wild-type control mice at 2 and 18 months of age (n=10 per group, equal sex distribution) can be used. Latency, path length, and proximity scores serve as measures of learning. Sensorimotor ability can be compared between groups and any observed differences in performance related differences will be teased apart from those pertaining to behavioral inflexibility. This type of memory test is a reliable method to assess deficits in contextual memory and discreet cued memory in mice, and is thought to be model explicit memory that appears to involve the hippocampus. In situations where behavioral differences are present and significant in the younger cohort, the oldest group will be used only for pathological studies; and in situations where no significant differences are observed in learning and memory behaviors between the two younger groups, behavior in older animals will then be assessed.

We have shown that mice lacking of β2M show enhanced learning and memory due to an increase in stem cell number and neurogenesis. The inhibitory effect observed in NPCs after exposure to β2M can be dependent on the relative changes in β2M levels observed with age rather than absolute levels. Therefore, younger animals normally express low levels of systemic β2M, and hence they may not exhibit robust changes in cognitive function in response to the absence of β2M; on the other hand, older β2M knock-out animals may exhibit a significant enhancement in learning and memory because the larger age-related relative increase in systemic β2M levels compared to wild-type controls has been abolished.

Although the number of proliferating NPCs increases in young β2M null mice, it may occur that a significant and sizeable increase in neurogenesis may not be observed in young adult mice. This would suggest an age-dependent regulatory role for β2M in adult neurogenesis: at younger ages, the abrogation of β2M can enhance the pool of quiescent stem cells, rather than actively differentiating progenitors, and provide a readily available source of stem cells that can become active at older ages.

The references cited herein and throughout the specification and examples are herein incorporated by reference in their entirety.

### REFERENCES

1. Nomura, T., et al., Beta2-microglobulin promotes the growth of human renal cell carcinoma through the activation of the protein kinase A, cyclic AMP-responsive element-binding protein, and vascular endothelial growth factor axis. Clin Cancer Res, 2006. 12(24): p. 7294-305.
2. Nomura, T., et al., Targeting beta2-microglobulin mediated signaling as a novel therapeutic approach for human renal cell carcinoma. J Urol, 2007. 178(1): p. 292-300.
3. Conboy, I. M., et al., Rejuvenation of aged progenitor cells by exposure to a young systemic environment. Nature, 2005. 433(7027): p. 760-4.
4. Klempin, F. and G. Kempermann, Adult hippocampal neurogenesis and aging. Eur Arch Psychiatry Clin Neurosci, 2007. 257(5): p. 271-80.
5. Lois, C. and A. Alvarez-Buylla, Proliferating subventricular zone cells in the adult mammalian forebrain can differentiate into neurons and glia. Proc Natl Acad Sci USA, 1993. 90(5): p. 2074-7.
6. Morshead, C. M., et al., Neural stem cells in the adult mammalian forebrain: a relatively quiescent subpopulation of subependymal cells. Neuron, 1994. 13(5): p. 1071-82.
7. Doetsch, F., A niche for adult neural stem cells. Curr Opin Genet Dev, 2003. 13(5): p. 543-50.
8. Eriksson, P. S., et al., Neurogenesis in the adult human hippocampus. Nat Med, 1998. 4(11): p. 1313-7.
9. Gould, E., et al., Hippocampal neurogenesis in adult Old World primates. Proc Natl Acad Sci USA, 1999. 96(9): p. 5263-7.
10. Kornack, D. R. and P. Rakic, Continuation of neurogenesis in the hippocampus of the adult macaque monkey. Proc Natl Acad Sci USA, 1999. 96(10): p. 5768-73.
11. Gage, F. H., Mammalian neural stem cells. Science, 2000. 287(5457): p. 1433-8.
12. Alvarez-Buylla, A. and D. A. Lim, For the long run: maintaining germinal niches in the adult brain. Neuron, 2004. 41(5): p. 683-6.
13. Scotto-Lomassese, S., et al., Suppression of adult neurogenesis impairs olfactory learning and memory in an adult insect. J Neurosci, 2003. 23(28): p. 9289-96.
14. Enwere, E., et al., Aging results in reduced epidermal growth factor receptor signaling, diminished olfactory neurogenesis, and deficits in fine olfactory discrimination. J Neurosci, 2004. 24(38): p. 8354-65.
15. Leuner, B., E. Gould, and T. J. Shors, Is there a link between adult neurogenesis and learning? Hippocampus, 2006. 16(3): p. 216-24.
16. Dupret, D., et al., Spatial learning depends on both the addition and removal of new hippocampal neurons. PLoS Biol, 2007. 5(8): p. e214.
17. Dupret, D., et al., Spatial relational memory requires hippocampal adult neurogenesis. PLoS ONE, 2008. 3(4): p. e1959.
18. Palmer, T. D., A. R. Willhoite, and F. H. Gage, Vascular niche for adult hippocampal neurogenesis. J Comp Neurol, 2000. 425(4): p. 479-94.
19. Capela, A. and S. Temple, LeX is expressed by principle progenitor cells in the embryonic nervous system, is secreted into their environment and binds Wnt-1. Dev Biol, 2006. 291(2): p. 300-13.
20. Louissaint, A., Jr., et al., Coordinated interaction of neurogenesis and angiogenesis in the adult songbird brain. Neuron, 2002. 34(6): p. 945-60.
21. Mercier, F., J. T. Kitasako, and G. I. Hatton, Anatomy of the brain neurogenic zones revisited: fractones and the fibroblast/macrophage network. J Comp Neurol, 2002. 451(2): p. 170-88.
22. Shen, Q., et al., Endothelial cells stimulate self-renewal and expand neurogenesis of neural stem cells. Science, 2004. 304(5675): p. 1338-40.
23. Monje, M. L., H. Toda, and T. D. Palmer, Inflammatory blockade restores adult hippocampal neurogenesis. Science, 2003. 302(5651): p. 1760-5.
24. Currle, D. S. and R. J. Gilbertson, The niche revealed. Cell Stem Cell, 2008. 3(3): p. 234-6.
25. Tavazoie, M., et al., A specialized vascular niche for adult neural stem cells. Cell Stem Cell, 2008. 3(3): p. 279-88.
26. Mirzadeh, Z., et al., Neural stem cells confer unique pinwheel architecture to the ventricular surface in neurogenic regions of the adult brain. Cell Stem Cell, 2008. 3(3): p. 265-78.
27. Shen, Q., et al., Adult SVZ stem cells lie in a vascular niche: a quantitative analysis of niche cell-cell interactions. Cell Stem Cell, 2008. 3(3): p. 289-300.
28. Rando, T. A., Stem cells, ageing and the quest for immortality. Nature, 2006. 441(7097): p. 1080-6.
29. Morrison, S. J., et al., The aging of hematopoietic stem cells. Nat Med, 1996. 2(9): p. 1011-6.
30. Kuhn, H. G., H. Dickinson-Anson, and F. H. Gage, Neurogenesis in the dentate gyrus of the adult rat: age-related decrease of neuronal progenitor proliferation. J Neurosci, 1996. 16(6): p. 2027-33.
31. Maslov, A. Y., et al., Neural stem cell detection, characterization, and age-related changes in the subventricular zone of mice. J Neurosci, 2004. 24(7): p. 1726-33.
32. Luo, J., et al., The aging neurogenic subventricular zone. Aging Cell, 2006. 5(2): p. 139-52.
33. Ahlenius, H., et al., Neural stem and progenitor cells retain their potential for proliferation and differentiation into functional neurons despite lower number in aged brain. J Neurosci, 2009. 29(14): p. 4408-19.
34. Lledo, P. M., M. Alonso, and M. S. Grubb, Adult neurogenesis and functional plasticity in neuronal circuits. Nat Rev Neurosci, 2006. 7(3): p. 179-93.
35. Alonso, M., et al., Olfactory discrimination learning increases the survival of adult-born neurons in the olfactory bulb. J Neurosci, 2006. 26(41): p. 10508-13.
36. Mirescu, C. and E. Gould, Stress and adult neurogenesis. Hippocampus, 2006. 16(3): p. 233-8.
37. Meshi, D., et al., Hippocampal neurogenesis is not required for behavioral effects of environmental enrichment. Nat Neurosci, 2006. 9(6): p. 729-31.
38. Temple, S. and A. Alvarez-Buylla, Stem cells in the adult mammalian central nervous system. Curr Opin Neurobiol, 1999. 9(1): p. 135-41.
39. Sommer, L. and M. Rao, Neural stem cells and regulation of cell number. Prog Neurobiol, 2002. 66(1): p. 1-18.
40. Molofsky, A. V., et al., Increasing p16INK4a expression decreases forebrain progenitors and neurogenesis during ageing. Nature, 2006. 443(7110): p. 448-52.
41. Molofsky, A. V., et al., Bmi-1 promotes neural stem cell self-renewal and neural development but not mouse growth and survival by repressing the p16Ink4a and p19Arf senescence pathways. Genes Dev, 2005. 19(12): p. 1432-7.
42. Renault, M. R., et al., FoxO3 regulates neural stem cell homeostasis. Cell Stem Cell (in press), 2009.
43. van Praag, H., et al., Running enhances neurogenesis, learning, and long-term potentiation in mice. Proc Natl Acad Sci USA, 1999. 96(23): p. 13427-31.
44. van Praag, H., G. Kempermann, and F. H. Gage, Running increases cell proliferation and neurogenesis in the adult mouse dentate gyrus. Nat Neurosci, 1999. 2(3): p. 266-70.
45. van Praag, H., et al., Exercise enhances learning and hippocampal neurogenesis in aged mice. J Neurosci, 2005. 25(38): p. 8680-5.
46. Yasuhara, T., et al., Lack of exercise, via hindlimb suspension, impedes endogenous neurogenesis. Neuroscience, 2007. 149(1): p. 182-91.
47. Van der Borght, K., et al., Exercise improves memory acquisition and retrieval in the Y-maze task: relationship with hippocampal neurogenesis. Behav Neurosci, 2007. 121(2): p. 324-34.
48. Levenson, C. W. and N. J. Rich, Eat less, live longer? New insights into the role of caloric restriction in the brain. Nutr Rev, 2007. 65(9): p. 412-5.
49. Fontan-Lozano, A., et al., Caloric restriction increases learning consolidation and facilitates synaptic plasticity through mechanisms dependent on NR2B subunits of the NMDA receptor. J Neurosci, 2007. 27(38): p. 10185-95.
50. Wagner, J. P., I. B. Black, and E. DiCicco-Bloom, Stimulation of neonatal and adult brain neurogenesis by subcutaneous injection of basic fibroblast growth factor. J Neurosci, 1999. 19(14): p. 6006-16.
51. Trejo, J. L., E. Carro, and I. Torres-Aleman, Circulating insulin-like growth factor I mediates exercise-induced increases in the number of new neurons in the adult hippocampus. J Neurosci, 2001. 21(5): p. 1628-34.
52. Jin, K., et al., Vascular endothelial growth factor (VEGF) stimulates neurogenesis in vitro and in vivo. Proc Natl Acad Sci USA, 2002. 99(18): p. 11946-50.
53. Fabel, K., et al., VEGF is necessary for exercise-induced adult hippocampal neurogenesis. Eur J Neurosci, 2003. 18(10): p. 2803-12.
54. Darnaudery, M., et al., Insulin-like growth factor 1 reduces age-related disorders induced by prenatal stress in female rats. Neurobiol Aging, 2006. 27(1): p. 119-27.
55. Lichtenwalner, R. J., et al., Intracerebroventricular infusion of insulin-like growth factor-I ameliorates the age-related decline in hippocampal neurogenesis. Neuroscience, 2001. 107(4): p. 603-13.
56. Pereira, A. C., et al., An in vivo correlate of exercise-induced neurogenesis in the adult dentate gyrus. Proc Natl Acad Sci USA, 2007. 104(13): p. 5638-43.
57. Gejyo, F., et al., Beta 2-microglobulin: a new form of amyloid protein associated with chronic hemodialysis. Kidney Int, 1986. 30(3): p. 385-90.
58. Relini, A., et al., Collagen plays an active role in the aggregation of beta2-microglobulin under physiopathological conditions of dialysis-related amyloidosis. J Biol Chem, 2006. 281(24): p. 16521-9.
59. Relini, A., et al., Heparin strongly enhances the formation of beta2-microglobulin amyloid fibrils in the presence of type I collagen. J Biol Chem, 2008. 283(8): p. 4912-20.
60. Murray, A. M., Cognitive impairment in the aging dialysis and chronic kidney disease populations: an occult burden. Adv Chronic Kidney Dis, 2008. 15(2): p. 123-32.
61. Corlin, D. B., et al., Quantification of cleaved beta2-microglobulin in serum from patients undergoing chronic hemodialysis. Clin Chem, 2005. 51(7): p. 1177-84.
62. Carrette, O., et al., A panel of cerebrospinal fluid potential biomarkers for the diagnosis of Alzheimer's disease. Proteomics, 2003. 3(8): p. 1486-94.
63. Gussow, D., et al., The human beta 2-microglobulin gene. Primary structure and definition of the transcriptional unit. J Immunol, 1987. 139(9): p. 3132-8.
64. Bjorkman, P. J. and P. Parham, Structure, function, and diversity of class I major histocompatibility complex molecules. Annu Rev Biochem, 1990. 59: p. 253-88.
65. Pedersen, L. O., et al., The interaction between beta 2-microglobulin (beta 2m) and purified class-I major histocompatibility (MHC) antigen. Scand J Immunol, 1994. 39(1): p. 64-72.
66. Boulanger, L. M. and C. J. Shatz, Immune signalling in neural development, synaptic plasticity and disease. Nat Rev Neurosci, 2004. 5(7): p. 521-31.
67. Wu, C. H., et al., beta(2)-microglobulin induces apoptosis in HL-60 human leukemia cell line and its multidrug resistant variants overexpressing MRP1 but lacking Bax or overexpressing P-glycoprotein. Oncogene, 2001. 20(48): p. 7006-20.
68. Menaa, C., E. Esser, and S. M. Sprague, Beta2-microglobulin stimulates osteoclast formation. Kidney Int, 2008. 73(11): p. 1275-81.
69. Huang, W. C., et al., beta2-microglobulin is a signaling and growth-promoting factor for human prostate cancer bone metastasis. Cancer Res, 2006. 66(18): p. 9108-16.
70. Centrella, M., T. L. McCarthy, and E. Canalis, Beta 2-microglobulin enhances insulin-like growth factor I receptor levels and synthesis in bone cell cultures. J Biol Chem, 1989. 264(31): p. 18268-71.
71. Due, C., M. Simonsen, and L. Olsson, The major histocompatibility complex class I heavy chain as a structural subunit of the human cell membrane insulin receptor: implications for the range of biological functions of histocompatibility antigens. Proc Natl Acad Sci USA, 1986. 83(16): p. 6007-11.
72. Rall, G. F., L. Mucke, and M. B. Oldstone, Consequences of cytotoxic T lymphocyte interaction with major histocompatibility complex class I-expressing neurons in vivo. J Exp Med, 1995. 182(5): p. 1201-12.
73. Mucke, L., et al., Rapid activation of astrocyte-specific expression of GFAP-lacZ transgene by focal injury. New Biol, 1991. 3(5): p. 465-74.
74. Grabowska, A. and L. A. Lampson, MHC expression in nonlymphoid tissues of the developing embryo: strongest class I or class II expression in separate populations of potential antigen-presenting cells in the skin, lung, gut, and inter-organ connective tissue. Dev Comp Immunol, 1995. 19(5): p. 425-50.
75. Linda, H., et al., Expression of MHC class I and beta2-microglobulin in rat spinal motoneurons: regulatory influences by IFN-gamma and axotomy. Exp Neurol, 1998. 150(2): p. 282-95.
76. Linda, H., et al., Expression of MHC class I heavy chain and beta2-microglobulin in rat brainstem motoneurons and nigral dopaminergic neurons. J Neuroimmunol, 1999. 101(1): p. 76-86.
77. Loconto, J., et al., Functional expression of murine V2R pheromone receptors involves selective association with the M10 and M1 families of MHC class Ib molecules. Cell, 2003. 112(5): p. 607-18.
78. Neumann, H., et al., Induction of MHC class I genes in neurons. Science, 1995. 269(5223): p. 549-52.
79. Corriveau, R. A., G. S. Huh, and C. J. Shatz, Regulation of class I MHC gene expression in the developing and mature CNS by neural activity. Neuron, 1998. 21(3): p. 505-20.
80. Corriveau, R. A., G. S. Huh, and C. J. Shatz, Regulation of Class I MHC gene expression in the developing and mature CNS by neural activity. Neuron, 1998. 21: p. 505-520.
81. Huh, G. S., et al., Functional requirement for class I MHC in CNS development and plasticity. Science, 2000. 290(5499): p. 2155-9.
82. Linda, H., et al., Expression of MHC class I and b2-microglobulin in rat spinal motoneurons: Regulatory influences by IFN-gamma and axotomy. Exp. Neurol., 1998. 150: p. 282-295.
83. Oliveira, A. L., et al., A role for MHC class I molecules in synaptic plasticity and regeneration of neurons after axotomy. Proc Natl Acad Sci USA, 2004. 101(51): p. 17843-8.
84. Ulfhake, B., et al., Regulation of neurotrophin signaling in aging sensory and motoneurons: dissipation of target support? Mol Neurobiol, 2000. 21(3): p. 109-35.
85. Kullberg, S., et al., Decreased axosomatic input to motoneurons and astrogliosis in the spinal cord of aged rats. J Gerontol A Biol Sci Med Sci, 1998. 53(5): p. B369-79.
86. Edstrom, E., et al., MHC class I, beta2 microglobulin, and the INF-gamma receptor are upregulated in aged motoneurons. J Neurosci Res, 2004. 78(6): p. 892-900.
87. Zou, H. and T. Hastie, Regularization and variable selection via the elastic net. Journal of the Royal Statistical Society: Series B (Statistical Methodology), 2005. 67(2): p. 301-320.
88. Tusher, V. G., R. Tibshirani, and G. Chu, Significance analysis of microarrays applied to the ionizing radiation response. Proc Natl Acad Sci USA, 2001. 98(9): p. 5116-21.
89. Garza, J. C., et al., Leptin increases adult hippocampal neurogenesis in vivo and in vitro. J Biol Chem, 2008. 283(26): p. 18238-47.
90. Ajami, B., et al., Local self-renewal can sustain CNS microglia maintenance and function throughout adult life. Nat Neurosci, 2007. 10(12): p. 1538-43.
91. Giorgetti, S., et al., beta2-Microglobulin is potentially neurotoxic, but the blood brain barrier is likely to protect the brain from its toxicity. Nephrol Dial Transplant, 2009. 24(4): p. 1176-81.
92. Fontan-Lozano, A., et al., Molecular bases of caloric restriction regulation of neuronal synaptic plasticity. Mol Neurobiol, 2008. 38(2): p. 167-77.
93. Zhao, C., W. Deng, and F. H. Gage, Mechanisms and functional implications of adult neurogenesis. Cell, 2008. 132(4): p. 645-60.
94. Pickford, F., et al., The autophagy-related protein beclin 1 shows reduced expression in early Alzheimer disease and regulates amyloid beta accumulation in mice. J Clin Invest, 2008. 118(6): p. 2190-9.
95. Segal, R. A. and M. E. Greenberg, Intracellular signaling pathways activated by neurotrophic factors. Annu Rev Neurosci, 1996. 19: p. 463-89.
96. Yao, H., et al., The cyclic adenosine monophosphate-dependent protein kinase (PKA) is required for the sustained activation of mitogen-activated kinases and gene expression by nerve growth factor. J Biol Chem, 1998. 273(14): p. 8240-7.
97. Grewal, S. S., R. D. York, and P. J. Stork, Extracellular-signal-regulated kinase signalling in neurons. Curr Opin Neurobiol, 1999. 9(5): p. 544-53.
98. Shamloo, M., A. Rytter, and T. Wieloch, Activation of the extracellular signal-regulated protein kinase cascade in the hippocampal CA1 region in a rat model of global cerebral ischemic preconditioning. Neuroscience, 1999. 93(1): p. 81-8.
99. Hu, B. R., C. L. Liu, and D. J. Park, Alteration of MAP kinase pathways after transient forebrain ischemia. J Cereb Blood Flow Metab, 2000. 20(7): p. 1089-95.
100. Schaeffer, M., et al., Signaling through a novel domain of gp130 mediates cell proliferation and activation of Hck and Erk kinases. Mol Cell Biol, 2001. 21(23): p. 8068-81.
101. Xiao, Z., et al., Upregulation of Flk-1 by bFGF via the ERK pathway is essential for VEGF-mediated promotion of neural stem cell proliferation. Cell Res, 2007. 17(1): p. 73-9.
102. Blum, S., et al., A mitogen-activated protein kinase cascade in the CA1/CA2 subfield of the dorsal hippocampus is essential for long-term spatial memory. J Neurosci, 1999. 19(9): p. 3535-44.
103. Atkins, C. M., et al., The MAPK cascade is required for mammalian associative learning. Nat Neurosci, 1998. 1(7): p. 602-9.
104. Berman, D. E., et al., The role of identified neurotransmitter systems in the response of insular cortex to unfamiliar taste: activation of ERK1-2 and formation of a memory trace. J Neurosci, 2000. 20(18): p. 7017-23.
105. Wiggins, C. M., H. Band, and S. J. Cook, c-Cbl is not required for ERK1/2-dependent degradation of BimEL. Cell Signal, 2007. 19(12): p. 2605-11.
106. Ley, R., et al., Activation of the ERK1/2 signaling pathway promotes phosphorylation and proteasome-dependent degradation of the BH3-only protein, Bim. J Biol Chem, 2003. 278(21): p. 18811-6.
107. Sussan, T. E., et al., Tumor suppressor in lung cancer 1 (TSLC1) alters tumorigenic growth properties and gene expression. Mol Cancer, 2005. 4: p. 28.
108. Capela, A. and S. Temple, LeX/ssea-1 is expressed by adult mouse CNS stem cells, identifying them as nonependymal. Neuron, 2002. 35(5): p. 865-75.
109. DeLorey, T. M., et al., Influence of benzodiazepine binding site ligands on fear-conditioned contextual memory. Eur J Pharmacol, 2001. 426(1-2): p. 45-54.
110. DeLorey, T. M., et al., Mice lacking the beta3 subunit of the GABAA receptor have the epilepsy phenotype and many of the behavioral characteristics of Angelman syndrome. J Neurosci, 1998. 18(20): p. 8505-14.
111. Harris, D. L., et al., Determinants of recognition of ligands binding to benzodiazepine receptor/GABA(A) receptors initiating sedation. Eur J Pharmacol, 2000. 401(3): p. 271-87.
112. Fanselow, M. S., Contextual fear, gestalt memories, and the hippocampus. Behav Brain Res, 2000. 110(1-2): p. 73-81.
113. Luo, J., et al., Glia-dependent TGF-beta signaling, acting independently of the TH17 pathway, is critical for initiation of murine autoimmune encephalomyelitis. J Clin Invest, 2007. 117(11): p. 3306-15.
114. Luo, J., et al., Bioluminescence imaging of Smad signaling in living mice shows correlation with excitotoxic neurodegeneration. Proc Natl Acad Sci USA, 2006. 103(48): p. 18326-31.
115. Choi, S. H., et al., Non-cell-autonomous effects of presenilin 1 variants on enrichment-mediated hippocampal progenitor cell proliferation and differentiation. Neuron, 2008. 59(4): p. 568-80.
116. Shingo, T., et al., Erythropoietin regulates the in vitro and in vivo production of neuronal progenitors by mammalian forebrain neural stem cells. J Neurosci, 2001. 21(24): p. 9733-43.
117. Liu, X. S., et al., Angiopoietin 2 mediates the differentiation and migration of neural progenitor cells in the subventricular zone after stroke. J Biol Chem, 2009. 284(34): p. 22680-9.
118. Tao, Y., I. B. Black, and E. DiCicco-Bloom, In vivo neurogenesis is inhibited by neutralizing antibodies to basic fibroblast growth factor. J Neurobiol, 1997. 33(3): p. 289-96.

### REFERENCES

¹ Rando, T. A., Stem cells, ageing and the quest for immortality. Nature 441 (7097), 1080-1086 (2006).
² van Praag, H., Kempermann, G., & Gage, F. H., Running increases cell proliferation and neurogenesis in the adult mouse dentate gyrus. Nat Neurosci 2 (3), 266-270 (1999).
³ van Praag, H., Shubert, T., Zhao, C., & Gage, F. H., Exercise enhances learning and hippocampal neurogenesis in aged mice. J Neurosci 25 (38), 8680-8685 (2005).
⁴ Lee, J., Duan, W., Long, J. M., Ingram, D. K., & Mattson, M. P., Dietary restriction increases the number of newly generated neural cells, and induces BDNF expression, in the dentate gyrus of rats. J Mol Neurosci 15 (2), 99-108 (2000).
⁵ Lledo, P. M., Alonso, M., & Grubb, M. S., Adult neurogenesis and functional plasticity in neuronal circuits. Nat Rev Neurosci 7 (3), 179-193 (2006).
⁶ Imayoshi, I. et al., Roles of continuous neurogenesis in the structural and functional integrity of the adult forebrain. Nat Neurosci 11 (10), 1153-1161 (2008).
⁷ Dupret, D. et al., Spatial relational memory requires hippocampal adult neurogenesis. PLoS ONE 3 (4), e1959 (2008).
⁸ Gage, F. H., Mammalian neural stem cells. Science 287 (5457), 1433-1438 (2000).
⁹ Alvarez-Buylla, A. & Lim, D. A., For the long run: maintaining germinal niches in the adult brain. Neuron 41 (5), 683-686 (2004).
¹⁰ van Praag, H. et al., Functional neurogenesis in the adult hippocampus. Nature 415 (6875), 1030-1034 (2002).
¹¹ Villeda, S. & Wyss-Coray, T., Microglia—a wrench in the running wheel? Neuron 59 (4), 527-529 (2008).
¹² Palmer, T. D., Willhoite, A. R., & Gage, F. H., Vascular niche for adult hippocampal neurogenesis. J Comp Neurol 425 (4), 479-494 (2000).
¹³ Shen, Q. et al., Endothelial cells stimulate self-renewal and expand neurogenesis of neural stem cells. Science 304 (5675), 1338-1340 (2004).
¹⁴ Lie, D. C. et al., Wnt signalling regulates adult hippocampal neurogenesis. Nature 437 (7063), 1370-1375 (2005).
¹⁵ Molofsky, A. V. et al., Increasing p16INK4a expression decreases forebrain progenitors and neurogenesis during ageing. Nature 443 (7110), 448-452 (2006).
¹⁶ Kuhn, H. G., Dickinson-Anson, H., & Gage, F. H., Neurogenesis in the dentate gyrus of the adult rat: age-related decrease of neuronal progenitor proliferation. J Neurosci 16 (6), 2027-2033 (1996).
¹⁷ Conboy, I. M. et al., Rejuvenation of aged progenitor cells by exposure to a young systemic environment. Nature 433 (7027), 760-764 (2005).
¹⁸ Ajami, B., Bennett, J. L., Krieger, C., Tetzlaff, W., & Rossi, F. M., Local self-renewal can sustain CNS microglia maintenance and function throughout adult life. Nat Neurosci 10 (12), 1538-1543 (2007).
¹⁹ Garza, J. C., Guo, M., Zhang, W., & Lu, X. Y., Leptin increases adult hippocampal neurogenesis in vivo and in vitro. J Biol Chem 283 (26), 18238-18247 (2008).
²⁰ Choi, S. H. et al., Non-cell-autonomous effects of presenilin 1 variants on enrichment-mediated hippocampal progenitor cell proliferation and differentiation. Neuron 59 (4), 568-580 (2008).
²¹ Couillard-Despres, S. et al., In vivo optical imaging of neurogenesis: watching new neurons in the intact brain. Mol Imaging 7 (1), 28-34 (2008).
²² Currle, D. S. & Gilbertson, R. J., The niche revealed. Cell Stem Cell 3 (3), 234-236 (2008).
²³ Tavazoie, M. et al., A specialized vascular niche for adult neural stem cells. Cell Stem Cell 3 (3), 279-288 (2008).
²⁴ Shen, Q. et al., Adult SVZ stem cells lie in a vascular niche: a quantitative analysis of niche cell-cell interactions. Cell Stem Cell 3 (3), 289-300 (2008).
²⁵ Ray, S. et al., Classification and prediction of clinical Alzheimer's diagnosis based on plasma signaling proteins. Nat Med (2007).
²⁶ Huh, G. S. et al., Functional requirement for class I MHC in CNS development and plasticity. Science 290 (5499), 2155-2159 (2000).
²⁷ Bjorkqvist, M. et al., A novel pathogenic pathway of immune activation detectable before clinical onset in Huntington's disease. J Exp Med 205 (8), 1869-1877 (2008).
²⁸ Luo, J. et al., Glia-dependent TGF-beta signaling, acting independently of the TH17 pathway, is critical for initiation of murine autoimmune encephalomyelitis. J Clin Invest 117 (11), 3306-3315 (2007).
²⁹ Couillard-Despres, S. et al., Human in vitro reporter model of neuronal development and early differentiation processes. BMC Neurosci 9, 31 (2008).
³⁰ Buckwalter, M. S. et al., Chronically increased transforming growth factor-beta1 strongly inhibits hippocampal neurogenesis in aged mice. Am J Pathol 169 (1), 154-164 (2006).

1. A method of increasing neurogenesis in a subject diagnosed with reduced cognitive function the method comprising administering to the subject diagnosed with reduced cognitive function a composition comprising a CCL11 neutralizing agent.

2. The method of claim 1, wherein the CCL11 neutralizing agent is a neutralizing antibody or RNA interfering agent against CCL11.

3. The method of claim 1, wherein the subject is human.

4. The method of claim 1 further comprising a step of assaying the subject for increased expression of CCL11.

5. The method of claim 4, wherein the assaying is performed using a biological sample selected from blood, serum, plasma, cerebrospinal fluid, or urine.

6. The method of claim 1, wherein the decreased cognitive function is associated with a neurodegenerative disease.

7. The method of claim 1, wherein the neurodegenerative disease is Alzheimer's disease, Parkinson's disease, Amyotrophic lateral sclerosis or neuroinflammatory disease.
